(19) United States
(12) Patent Application Publication
(10) Pub. No.: US 2024/0327655 A1
(43) Pub. Date: Oct. 3, 2024

(54) DYE-LABELED POLYMERS AND METHODS FOR PREPARING SAME

(71) Applicant: DEBOGY MOLECULAR, INC., Battle Creek, MI (US)

(72) Inventors: Othman BOULOUSSA, Bordeaux (FR); Maha ALQURAFI, Farmington, CT (US)

(73) Assignee: DEBOGY MOLECULAR, INC., Battle Creek, MI (US)

(21) Appl. No.: 18/551,558

(22) PCT Filed: Mar. 25, 2022
(86) PCT No.: PCT/US2022/022043

§ 371 Date: Sep. 20, 2023

Related U.S. Application Data
(60) Provisional application No. 63/166,121, filed on Mar. 25, 2021.
(60) Provisional application No. 63/323,034, filed on Mar. 23, 2022.

(51) Int. Cl. C09D 5/22; A01N 33/12; A01N 43/40; A01P 1/00; A01P 3/00; C07D 311/18; C07F 7/18; C08F 8/18; C08F 8/30; C08G 73/02; C09B 69/10; C09D 5/14; C09D 7/41; C09D 125/18; C09D 139/08; C09D 179/02; C09K 11/06

(52) U.S. Cl. CPC C09D 5/22; A01N 33/12; A01N 43/40; A01P 1/00; A01P 3/00; C07D 311/18; C07F 7/1804; C08F 8/18; C08F 8/30; C08G 73/0206; C09B 69/109; C09D 5/14; C09D 7/41; C09D 125/18; C09D 139/08; C09D 179/02; C09K 11/06; C09K 2211/1433; C09K 2211/145; C09K 2211/1466

(57) ABSTRACT

Biocidal polymers and compounds comprising detectable moieties, coatings including same, and methods of making and using the same for controlling the growth of at least one bacteria, fungi, protozoa, or virus are disclosed.

### CROSS REFERENCE TO RELATED APPLICATIONS

The present application claims priority to U.S. Provisional Patent Application Nos. 63/166,121, filed Mar. 25, 2021, and 63/323,034, filed Mar. 23, 2022, the contents of all of which are incorporated by reference herein in their entireties.

### FIELD

The disclosure relates generally to novel polymers and compounds useful for preparing detectable coatings having both biocidal and biocompatibility properties, methods of preparation of same, and methods of grafting same on surfaces to reduce biofilm formation.

### BACKGROUND

Biocidal polymers are becoming increasingly important in order to contain and control the spread of infectious pathogens in a variety of health and industrial applications. To this end, biocidal polymers have been developed for use in solution form as well as to incorporate biocidal activity onto materials via coatings.

It would be highly desirable to have a solution of a biocidal polymer having both biocidal and biocompatibility properties for a prolonged storage period. It is, therefore, advisable to have a ready-to-use biocidal product which prevents fast reticulation in volume and thereby prolonging storage period.

### BRIEF SUMMARY

In one aspect, the disclosure provides polymers comprising one or more detectable moieties. In some embodiments, the detectable moiety is one or more selected from a fluorescent moiety, a phosphorescent moiety, and a luminescent moiety. In some embodiments, the detectable moiety is a fluorescent moiety selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety.

In some embodiments, the detectable moiety is a moiety of formula (I), formula (II), or formula (VII). In some embodiments, the detectable moiety is selected from formula (10), formula (20), formula (21), and formula (70). In some embodiments, R^{3a} and R^{3b} are each ethyl.

In some embodiments, the polymer comprises at least one moiety of formula (Iaa), at least one moiety of formula (3), and at least one moiety of formula (4). In some embodiments, the polymer comprises at least one moiety of formula (Ia), at least one moiety of formula (3), and at least one moiety of formula (4). In some embodiments, the polymer comprises at least one moiety of formula (VIIa), at least one moiety of formula (3), and at least one moiety of formula (4).

In some embodiments, the polymer comprises at least one moiety of formula (VIIb), at least one moiety of formula (3), and at least one moiety of formula (4). In some embodiments, in formula (4), v is 3 and each R³ is methoxy. In some embodiments, the moieties of formula (Ia), moieties of formula (3), and moieties of formula (4) are present in a molar ratio of x:y:z, wherein 0.1≤x≤0.2:0.6≤y≤0.8:0.1≤z≤0.2, and x+y+z=1; wherein the moieties of formula (Iaa), moieties of formula (3), and moieties of formula (4) are present in a molar ratio of x:y:z, wherein 0.1≤x≤0.2:0.6≤y≤0.8:0.1≤z≤0.2, and x+y+z=1; wherein the moieties of formula (VIIa), moieties of formula (3), and moieties of formula (4) are present in a molar ratio of x:y:z, wherein 0.1≤x≤0.2:0.6≤y≤0.8:0.1≤z≤0.2, and x+y+z=1; and/or wherein the moieties of formula (VIIb), moieties of formula (3), and moieties of formula (4) are present in a molar ratio of x:y:z, wherein 0.1≤x≤0.2:0.6≤y≤0.8:0.1≤z≤0.2, and x+y+z=1.

In some embodiments, the polymer comprises at least one moiety of formula (Iaa), at least one moiety of formula (5), and at least one moiety of formula (6). In some embodiments, v=the polymer comprises at least one moiety of formula (Ia), at least one moiety of formula (5), and at least one moiety of formula (6). In some embodiments, the polymer comprises at least one moiety of formula (VIIa), at least one moiety of formula (5), and at least one moiety of formula (6). In some embodiments, the polymer comprises at least one moiety of formula (VIIb), at least one moiety of formula (5), and at least one moiety of formula (6).

In some embodiments, the moieties of formula (Iaa), moieties of formula (5), and moieties of formula (6) are present in a molar ratio of x:y:z, wherein 0.1≤x≤0.2:0.1≤y≤0.2:0.6≤z≤0.8, and x+y+z=1; the moieties of formula (Ia), moieties of formula (5), and moieties of formula (6) are present in a molar ratio of x:y:z, wherein 0.1≤x≤0.2:0.1≤y≤0.2:0.6 K z≤0.8, and x+y+z=1; the moieties of formula (VIIa), moieties of formula (5), and moieties of formula (6) are present in a molar ratio of x:y:z, wherein 0.1≤x≤0.2:0.1≤y≤0.2:0.6≤z≤0.8, and x+y+z=1; and/or the moieties of formula (VIIb), moieties of formula (5), and moieties of formula (6) are present in a molar ratio of x:y:z, wherein 0.1≤x≤0.2:0.1≤y≤0.2:0.6≤z≤0.8, and x+y+z=1.18.

In some embodiments, the polymer comprises at least one moiety of formula (Ibb) at least one moiety of formula (7), and at least one moiety of formula (8). In some embodiments, the polymer comprises at least one moiety of formula (Ib) at least one moiety of formula (7), and at least one moiety of formula (8). In some embodiments, the polymer comprises at least one moiety of formula (IIaa), at least one moiety of formula (7), and at least one moiety of formula (8). In some embodiments, the polymer comprises at least one moiety of formula (IIa), at least one moiety of formula (7), and at least one moiety of formula (8). In some embodiments, in formula (Ib), each R³ is ethyl. In some embodiments, in formula (7), each R³ is methyl. In some embodiments, in formula (8), each R³ is methyl, v is 3, and each R⁴ is methoxy.

In some embodiments, the moieties of formula (Ibb), moieties of formula (7), and moieties of formula (8) are present in a molar ratio of x:y:z, wherein 0.1≤x≤0.2:0.6≤y≤0.8:0.1≤z≤0.2, and x+y+z=1; the moieties of formula (Ibb), moieties of formula (7), and moieties of formula (8) are present in a molar ratio of x:y:z, wherein 0.1≤x≤0.2:0.6≤y≤0.8:0.1≤z≤0.2, and x+y+z=1; the moieties of formula (IIaa), moieties of formula (7), and moieties of formula (8) are present in a molar ratio of x:y:z, wherein 0.1≤x≤0.2:0.6≤y≤0.8:0.1≤z≤0.2, and x+y+z=1; and/or the moieties of formula (IIa), moieties of formula (7), and moieties of formula (8) are present in a molar ratio of x:y:z, wherein 0.1≤x≤0.2:0.6≤y≤0.8:0.1≤z≤0.2, and x+y+z=1. In some embodiments, the polymer comprises at least one moiety of formula (IIaa), at least one moiety of formula (7), and at least one moiety of formula (13). In some embodiments, the polymer comprises at least one moiety of formula (IIa), at least one moiety of formula (7), and at least one moiety of formula (13). In some embodiments, the moieties of formula (IIaa), moieties of formula (7), and moieties of formula (13) are present in a molar ratio of x:y:z, wherein 0.1≤x≤0.2:0.6≤y≤0.8:0.1≤z≤0.2, and x+y+z=1; and/or the moieties of formula (IIa), moieties of formula (7), and moieties of formula (13) are present in a molar ratio of x:y:z, wherein 0.1≤x≤0.2:0.6≤y≤0.8:0.1≤z≤0.2, and x+y+z=1.

In some embodiments, the polymer comprises a polyethylenimine (PEI) polymer comprising at least one moiety of formula (IXa), formula (IXb), or formula (IXc). In some embodiments, L is selected from

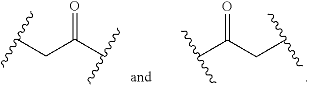 [SMILES: *CC(*)=O ; *CC(*)=O]

In some embodiments, the PEI polymer comprises at least one moiety of formula (IXa1), formula (IXb1), or formula (IXc1): IN some embodiments, the PEI polymer comprises at least one moiety of formula (IXa2), formula (IXb2), or formula (IXc2). In some embodiments, the PEI polymer is fully quaternized, optionally fully methylated.

In some embodiments, the PEI polymer further comprises one or more optionally substituted C₄-C₂₂ alkyl groups, optionally C₆ alkyl groups or C₁₀ alkyl group, and one or more

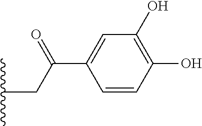 [SMILES: *CC(=O)c1ccc(O)c(O)c1]

groups. In some embodiments, the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of

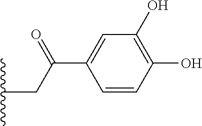 [SMILES: *CC(=O)c1ccc(O)c(O)c1]

groups is x:y:z, wherein about 0.05≤x≤0.4:0.2≤y≤0.9:0.05≤z≤0.4, optionally 0.1:0.8:0.1. In some embodiments, the PEI polymer further comprises one or more optionally substituted C₄-C₂₂ alkyl groups, optionally C₆ alkyl groups or C₁₀ alkyl group, and one or more moieties of formula (9):

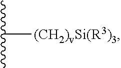

wherein each R³ is independently optionally substituted alkoxy; and v is an integer from 3 to 10, optionally

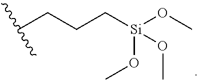 [SMILES: *CCC[Si](OC)(OC)OC]

In some embodiments, the PEI polymer is fully quaternized, optionally fully methylated. In some embodiments, the PEI polymer further comprises one or more optionally substituted C₄-C₂₂ alkyl groups, optionally C₆ alkyl groups or C₁₀ alkyl group, and one or more

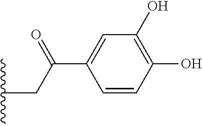 [SMILES: *CC(=O)c1ccc(O)c(O)c1]

groups. In some embodiments, the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of

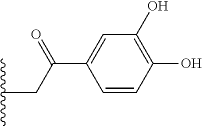 [SMILES: *CC(=O)c1ccc(O)c(O)c1]

groups is x:y:z, wherein about 0.05≤x≤0.4:0.2≤y≤0.9:0.05≤z≤0.4, optionally 0.1:0.8:0.1. In some embodiments, the PEI polymer further comprises one or more optionally substituted C₄-C₂₂ alkyl groups, optionally C₆ alkyl groups or C₁₀ alkyl group, and one or more moieties of formula (9):

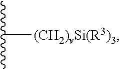

wherein each R³ is independently optionally substituted alkoxy; and v is an integer from 3 to 10, optionally

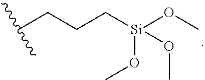 [SMILES: *CCC[Si](OC)(OC)OC]

In some embodiments, the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number one or more moieties of formula (9), optionally

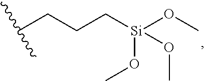 [SMILES: *CCC[Si](OC)(OC)OC]

is x:y:z, wherein about 0.05≤x≤0.4:0.2≤y≤0.9:0.05≤z≤0.4, optionally 0.1:0.8:0.1. In some embodiments, the PEI polymer a PEI polymer comprises at least one moiety of formula (IXe), or substructures thereof. the PEI polymer a PEI polymer comprises at least one moiety of formula (IXf), or substructures thereof. the PEI polymer a PEI polymer comprises at least one moiety of formula (IXg), or substructures thereof. the PEI polymer a PEI polymer comprises at least one moiety of formula (IXh), or substructures thereof

In some embodiments, in formula (IXg), the molar ratio of the number of R⁵ moieties of formula (I) to the number of R⁵ optionally substituted alkyl groups to the number of R⁵ catechol moieties of

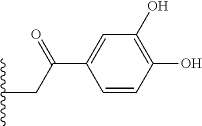 [SMILES: *CC(=O)c1ccc(O)c(O)c1]

is about 0.1:0.8:0.1; wherein in formula (IXf), the molar ratio of the number of R⁵ moieties of formula (VII) to the number of R⁵ optionally substituted alkyl groups to the number of R⁵ catechol moieties of

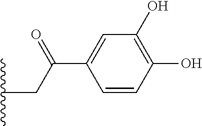 [SMILES: *CC(=O)c1ccc(O)c(O)c1]

is about 0.1:0.8:0.1; wherein in formula (IXg), the molar ratio of the number of R⁵ moieties of formula (I) to the number of R⁵ optionally substituted alkyl groups to the number of R⁵ groups of formula (9) is about 0.1:0.8:0.1; and/or wherein in formula (IXh), the molar ratio of the number of R⁵ moieties of formula (VII) to the number of R⁵ optionally substituted alkyl groups to the number of R⁵ groups of formula (9) is about 0.1:0.8:0.1.

In another aspect, the disclosure provides a compound comprising one or more detectable moieties. In some embodiments, the detectable moiety is one or more moieties selected from a fluorescent moiety, a phosphorescent moiety, and a luminescent moiety. In some embodiments, the detectable moiety is a fluorescent moiety selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety. In some embodiments, the detectable moiety is a moiety of formula (I) or formula (II). In some embodiments, the detectable moiety is selected from formula (10) and formula (20). In some embodiments, R^{3a} and R^{3b} are each ethyl. In some embodiments, the compound of formula (I) is a compound of formula (Ic). In some embodiments, each R³ is methyl, v is 3, and each R⁴ is methoxy. In some embodiments, the compound of formula (II) is a compound of formula (IIa). In some embodiments, each R⁵ is ethyl. In some embodiments, the compound is a compound having formula (III). In some embodiments, the compound of formula (III) is a compound having formula (IIIa) or formula (IIIb). In some embodiments, the compound has the formula (300) or formula (301). In some embodiments, the compound is a compound of formula (IV). In some embodiments, the compound has formula (400). In some embodiments, the compound is a compound having formula (VI). In some embodiments, the compound of formula (VI) is a compound having formula (VIa) or formula (VIb). In some embodiments, the compound has the formula (600) or formula (600). In some embodiments, the compound is a compound having formula (VIII). In some embodiments, the compound of formula (VIII) is a compound having formula (VIIIa). In some embodiments, the compound has formula (800).

In another aspect, the disclosure provides a method of preparing a polymer comprising a detectable moiety, the method comprising treating a precursor polymer with a reactive detectable compound. In some embodiments, the detectable compound is covalently attached to the precursor polymer after treatment. In some embodiments, the detectable moiety comprises one or more moieties selected from a fluorescent moiety, a phosphorescent moiety, and a luminescent moiety. In some embodiments, the fluorescent moiety is selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety. In some embodiments, the reactive detectable compound is a compound of formula (100) or formula (200).

In another aspect, the disclosure provides a method of preparing a detectable coating, the method comprising depositing a biocidal polymer solution comprising one or more polymers of any one of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11) onto a surface. In some embodiments, the method comprises waiting a suitable period of time for the biocidal polymer solution to dry.

In another aspect, the disclosure provides a method of preparing a detectable coating, the method comprising depositing a biocidal polymer solution onto a surface, wherein the biocidal polymer solution comprises one or more polymers of any one of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11) and one or more compounds of formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210). In some embodiments, the method comprises waiting a period of time for the biocidal polymer solution to dry. In some embodiments, the compound is cross-linked to the polymer after drying.

In another aspect, the disclosure provides a detectable coating comprising one or more polymers of any one of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11).

In another aspect, the disclosure provides a detectable coating comprising one or more polymers of any one of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11) and one or more compounds of formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210). In some embodiments, the one or more polymers and the one or more compounds are present in a ratio of about 1:1 w/w. In some embodiments, the polymer is selected from PVP-silane-coalkyl-PVP and PVP-catechol-coalkyl-PVP. In some embodiments, the detectable coating emits light when exposed to light having a wavelength ranging from about 10 nm to about 400 nm.

### BRIEF DESCRIPTION OF THE FIGURES

FIG. 1 illustrates the structure of a dye-labeled graftable PVP-based biocidal polymer of the disclosure. 3-IPTMS=3-iodopropyltrimethoxysilane.

FIGS. 2A and 2B illustrate images of glass slides coated with either a dye-labeled PVP-based biocidal polymer of the disclosure (FIG. 2A) or control the fluorescence emitted by a dye-labeled PVP-based biocidal polymer of the disclosure (FIG. 2A) compared to a control (FIG. 2B). The treated slide exhibited markedly green fluorescence while the control did not shine under UV light.

FIGS. 3A and 3B illustrate non-limiting examples of syntheses of a fluorescent silane of the disclosure for use as a guest molecule. FIG. 3C illustrates an exemplary IR-spectrum of a fluorescent silane of the disclosure.

FIGS. 4A-4C illustrate successful grafting of compounds of the disclosure onto filter paper. FIG. 4A illustrates an image comparing filter paper samples following a fluorescein test: control (right) and treated (left) (treated with a fluorescent silane compound of the disclosure).

FIG. 4B illustrates an image comparing filter paper samples under UV light (365 nm): control (left) and treated (right) (treated with a detectable fluorescent silane compound of the disclosure).

FIG. 4C illustrates an image of filter papers treated with control (left) and fluorescent silane compound of the disclosure (right).

FIG. 5A illustrates an exemplary synthesis of a dye-labeled graftable catechol PVP-based biocidal polymer of the disclosure. FIG. 5B illustrates an illustrates an IR spectrum of the product prepared by the exemplary method of FIG. 5A.

FIGS. 6A and 6B illustrate non-limiting examples of syntheses of a fluorescent catechol compound of the disclosure for use as a guest molecule. FIG. 6C illustrates an IR spectrum of the product prepared by the exemplary method of FIG. 6C.

FIGS. 7A and 7B illustrate non-limiting examples of syntheses of a dye-labeled graftable catechol PVB-based biocidal polymer of the disclosure. FIG. 7A illustrates the preparation of a PVB-Silane Coumarin prepared from N,N-dimethylaminopropyltrimethoxysilane and 3-(diethylaminoacetyl) coumarin. FIG. 7B illustrates the preparation of a PVB-Silane Coumarin prepared from N,N-dimethylaminopropyltrimethoxysilane and 7-diethylamino-4-methylcoumarin.

FIGS. 8A and 8B illustrate an image of a glass slide coated with a mixture of PVP-silane-coalkyl-PVP and a fluorescent silane of the disclosure.

FIGS. 9A and 9B illustrate successful grafting of compounds of the disclosure onto filter paper. FIG. 9A illustrates an image comparing filter paper samples under UV light (365 nm): control (left) and treated (right) (treated with a detectable compound of the disclosure obtained from reacting 7-diethylamino-4-methylcoumarin (231 mg, 1 mmol) with 4-chloroacetylcatechol (186 mg, 1 mmol) in ethanol). FIG. 9B illustrates an image comparing filter paper samples following a fluorescein test: control (right) and treated (left) (treated with a detectable moiety from reacting 7-diethylamino-4-methylcoumarin (231 mg, 1 mmol) with 4-chloroacetylcatechol (186 mg, 1 mmol) in ethanol).

FIG. 10 illustrates an exemplary IR-spectrum of a detectable compound of the disclosure (catechol-coumarin) obtained from reacting 7-diethylamino-4-methylcoumarin (231 mg, 1 mmol) with 4-chloroacetylcatechol (186 mg, 1 mmol) in ethanol).

FIGS. 11A-11C illustrate successful grafting of polymers of the disclosure onto filter paper and glass. FIG. 11A illustrates an image comparing filter paper samples under UV light (365 nm): control (left) and treated (right) (treated with biocidal fluorescent quaternized polyvinylbenzylchloride of the disclosure). FIG. 11B illustrates an image comparing filter paper samples following a fluorescein test: control (right) and treated (left) (treated with biocidal fluorescent quaternized polyvinylbenzylchloride of the disclosure). FIG. 11C illustrates an image of a glass slide grafted with the biocidal fluorescent quaternized polyvinylbenzylchloride.

FIG. 12 illustrates an exemplary IR-spectrum of biocidal fluorescent quaternized polyvinylbenzylchloride of the disclosure.

FIGS. 13A-13D illustrate successful grafting of polymers of the disclosure onto filter paper. FIGS. 13A and 13C illustrate an image comparing filter paper samples under UV light (365 nm): control (left) and treated (right) (treated with biocidal fluorescent silane-based quaternized polyvinylpyridine of the disclosure). FIG. 13B illustrates an image comparing filter paper samples following a fluorescein test: control (right) and treated (left) (treated with biocidal fluorescent silane-based quaternized polyvinylpyridine of the disclosure). FIG. 13D illustrates an image comparing filter paper samples following a fluorescein test: control (left) and treated (right) (treated with biocidal fluorescent silane-based quaternized polyvinylpyridine of the disclosure).

FIGS. 14A-14D illustrate successful grafting of polymers of the disclosure onto filter paper. FIGS. 14A and 14C illustrate an image comparing filter paper samples under UV light (365 nm): control (left) and treated (right) (treated with biocidal fluorescent catechol-based quaternized polyvinylpyridine of the disclosure). FIG. 14B illustrates an image comparing filter paper samples following a fluorescein test: control (right) and treated (left) (treated with biocidal fluorescent catechol-based quaternized polyvinylpyridine of the disclosure). FIG. 14D illustrates an image comparing filter paper samples following a fluorescein test: control (left) and treated (right) (treated with biocidal fluorescent catechol-based quaternized polyvinylpyridine of the disclosure).

FIG. 15 illustrates a non-limiting example of a IR-spectrum of biocidal fluorescent silane-based quaternized polyvinylpyridine of the disclosure.

FIGS. 16A and 16B illustrate successful grafting of polymers of the disclosure onto filter paper. FIG. 16A illustrates an image comparing filter paper samples under UV light (365 nm): control (left) and treated (right) (treated with biocidal fluorescent silane-based quaternized methylpolyethylenimine of the disclosure). FIG. 16B illustrates an image comparing filter paper samples following a fluorescein test: control (right) and treated (left) (treated with biocidal silane-based quaternized methylpolyethylenimine of the disclosure).

FIG. 17 illustrates a non-limiting example of a IR-spectrum of biocidal fluorescent catechol-based quaternized methylpolyethylenimine of the disclosure.

FIGS. 18A and 18B illustrate successful grafting of polymers of the disclosure onto filter paper. FIG. 18A illustrates an image comparing filter paper samples under UV light (365 nm): control (left) and treated (right) (treated with biocidal fluorescent catechol-based quaternized methylpolyethylenimine of the disclosure). FIG. 18B illustrates an image comparing filter paper samples following a fluorescein test: control (right) and treated (left) (treated with biocidal fluorescent catechol-based quaternized methylpolyethylenimine of the disclosure).

FIG. 19 illustrates a non-limiting example of a IR-spectrum of biocidal fluorescent catechol-based quaternized methylpolyethylenimine of the disclosure.

FIGS. 20A and 20B illustrate successful grafting of compounds of the disclosure onto filter paper. FIG. 20A illustrates an image comparing filter paper samples under UV light (365 nm): control (left) and treated (right) (treated with an antimicrobial silane-based C18 quaternary ammonium compound of the disclosure). FIG. 20B illustrates an image comparing filter paper samples following a fluorescein test: control (right) and treated (left) (treated with an antimicrobial silane-based C18 quaternary ammonium compound of the disclosure).

FIG. 21 illustrates a non-limiting example of a IR-spectrum of an fluorescent silane-based C18 quaternary ammonium compound of the disclosure.

FIGS. 22A and 22B illustrate successful grafting of compounds of the disclosure onto filter paper. FIG. 22A illustrates an image comparing filter paper samples under UV light (365 nm): control (left) and treated (right) (treated with an antimicrobial catechol-based C18 quaternary ammonium compound of the disclosure). FIG. 22B illustrates an image comparing filter paper samples following a fluorescein test: control (right) and treated (left) (treated with an antimicrobial catechol-based C18 quaternary ammonium compound of the disclosure).

FIG. 23 illustrates a non-limiting example of a IR-spectrum of a fluorescent catechol-based C18 quaternary ammonium compound of the disclosure.

FIGS. 24A and 24B illustrate successful grafting of compounds of the disclosure onto filter paper. FIG. 24A illustrates an image comparing filter paper samples under UV light (365 nm): control (left) and treated (right) (treated with an antimicrobial dipodal silane-based C10 quaternary ammonium compound of the disclosure). FIG. 24B illustrates an image comparing filter paper samples following a fluorescein test: control (right) and treated (left) (treated with an antimicrobial dipodal silane-based C10 quaternary ammonium compound of the disclosure).

FIG. 25 illustrates a non-limiting example of a IR-spectrum of a dipodal silane-based C10 quaternary ammonium compound of the disclosure.

FIG. 26 illustrates a non-limiting example of a synthesis of a fluorescent dipodal silane coumarin compound of the disclosure for use as a guest molecule.

FIGS. 27A-27C illustrate experimental data related to compounds of the disclosure.

FIGS. 27A-27B illustrate successful grafting of compounds of the disclosure onto filter paper.

FIG. 27A illustrates an image comparing filter paper samples under UV light (365 nm): control (left) and treated (right) (treated with a fluorescent dipodal silane coumarin compound of the disclosure). FIG. 27B illustrates an image comparing filter paper samples following a fluorescein test: control (right) and treated (left) (treated with a fluorescent dipodal silane coumarin compound of the disclosure). FIG. 27C illustrates an exemplary IR-spectrum of a fluorescent dipodal silane coumarin compound of the disclosure.

FIG. 28 illustrates a non-limiting example of a synthesis of a fluorescent silane coumarin compound of the disclosure for use as a guest molecule.

FIGS. 29A-29C illustrate experimental data related to compounds of the disclosure.

FIGS. 29A-29B illustrate successful grafting of compounds of the disclosure onto filter paper.

FIG. 29A illustrates an image comparing filter paper samples under UV light (365 nm): control (left) and treated (right) (treated with a fluorescent silane coumarin compound of the disclosure).

FIG. 29B illustrates an image comparing filter paper samples following a fluorescein test: control (right) and treated (left) (treated with a fluorescent silane coumarin compound of the disclosure).

FIG. 29C illustrates an exemplary IR-spectrum of a fluorescent silane coumarin compound of the disclosure.

FIG. 30 illustrates a non-limiting example of a synthesis of a fluorescent dipodal silane coumarin compound of the disclosure for use as a guest molecule.

FIGS. 31A-31C illustrate experimental data related to compounds of the disclosure.

FIGS. 31A-31B illustrate successful grafting of compounds of the disclosure onto filter paper.

FIG. 31A illustrates an image comparing filter paper samples under UV light (365 nm): control (left) and treated (right) (treated with a fluorescent silane coumarin compound of the disclosure).

FIG. 31B illustrates an image comparing filter paper samples following a fluorescein test: control (right) and treated (left) (treated with a fluorescent silane coumarin compound of the disclosure).

FIG. 31C illustrates an exemplary IR-spectrum of a fluorescent silane coumarin compound of the disclosure.

FIG. 32A illustrates a non-limiting example of a synthesis of a methyl PEI coumarin catechol polymer of the disclosure. FIG. 32B illustrates an IR spectrum of a methyl PEI coumarin catechol polymer of the disclosure. FIGS. 32C-32D illustrate successful grafting of polymers of the disclosure onto filter paper. FIG. 32C illustrates an image comparing filter paper samples under UV light (365 nm): control (left) and treated (right) (treated with a methyl PEI coumarin catechol polymer of the disclosure). FIG. 32D illustrates an image comparing filter paper samples following a fluorescein test: control (left) and treated (right) (treated with a methyl PEI coumarin catechol polymer of the disclosure).

FIG. 33 illustrates a non-limiting example of a synthesis of a methyl PEI coumarin catechol polymer of the disclosure.

FIG. 34A illustrates a non-limiting example of a synthesis of a methyl PEI coumarin silyl polymer of the disclosure. FIG. 34B illustrates an IR spectrum of a methyl PEI coumarin silyl polymer of the disclosure. FIGS. 34C-34D illustrate successful grafting of polymers of the disclosure onto filter paper. FIG. 34C illustrates an image comparing filter paper samples under UV light (365 nm): control (left) and treated (right) (treated with a methyl PEI coumarin silyl polymer of the disclosure).

FIG. 35A illustrates a non-limiting example of a synthesis of a methyl PEI coumarin silyl polymer of the disclosure. FIGS. 35B-35D illustrate successful grafting of polymers of the disclosure onto filter paper. FIG. 35B illustrates an image comparing filter paper samples under UV light (365 nm): control (left) and treated (right) (treated with a methyl PEI coumarin silyl polymer of the disclosure).

FIG. 36A illustrates a non-limiting example of a synthesis of a PVP coumarin silyl polymer of the disclosure. FIG. 36B illustrates an IR spectrum of a PVP coumarin silyl polymer of the disclosure

FIG. 37 illustrates a non-limiting example of a synthesis of a PVP coumarin catechol polymer of the disclosure.

FIG. 38 illustrates a non-limiting example of a synthesis of a PVP coumarin silyl polymer of the disclosure.

FIG. 39A illustrates a non-limiting example of a synthesis of a PVP coumarin catechol polymer of the disclosure. FIG. 39B illustrates an IR spectrum of a PVP coumarin silyl polymer of the disclosure

FIG. 40 illustrates a non-limiting example of a synthesis of a PVBC coumarin silyl polymer of the disclosure.

FIG. 41 illustrates a non-limiting example of a synthesis of a PVBC coumarin catechol polymer of the disclosure.

### DETAILED DESCRIPTION

Unless defined otherwise, all technical and scientific terms used herein have the same meaning as is commonly understood by one of skill in the art to which this invention belongs. All patents and publications referred to herein are incorporated by reference in their entireties.

### Definitions

The term “biocide”, as used herein, means a chemical compound, a chemical composition, a chemical formulation which can kill or render harmless a microorganism exemplified by bacterium, yeast, protozoa, and fungi.

The term “statistical copolymer” as used herein is defined as a copolymer that is made up of more than one monomer, and in which the different monomer units are randomly distributed in the polymeric chain.

As used herein, the terms “graft” and “grafting” refer to the attachment of moieties onto a surface by forming covalent linkages between functional groups on the surface and the moiety.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R² of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Oxa” refers to the —O— radical.

“Oxo” refers to the ═O radical.

“Moiety” refers to a specific segment or functional group of a molecule. Chemical moieties are often recognized chemical entities embedded in or appended to a molecule.

A “leaving group or atom” is any group or atom that will, under selected reaction conditions, cleave from the starting material, thus promoting reaction at a specified site.

Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material.

The term “consisting of” excludes any element, step or material other than those specified in the claim and, in the latter instance, impurities ordinary associated with the specified material(s).

The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Polymers and Compounds

The catechol moiety may form bonds, e.g., covalent bonds, with hydroxylated substrates, and has been utilized as a linker. However, the catechol moiety has several drawbacks, especially its rapid polymerization at a basic pH and spontaneous oxidation to quinones.

As depicted in reaction sequence A of Scheme 1 below, reactions between a surface and a volume (e.g. a solution comprising the bromide compound) usually occur according to an S_{N}2 mechanism with the nucleophile almost always localized on the surface while the electrophile is located in the volume.

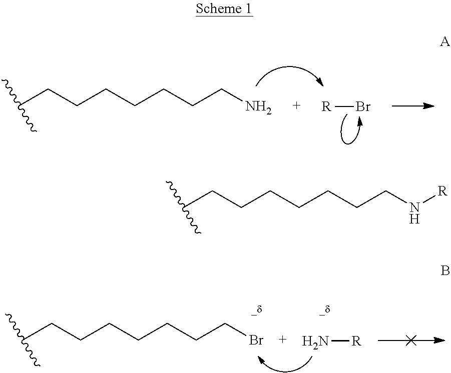

In contrast, the reaction sequence B of Scheme 1 cannot be completed due to electrostatic repulsions. The only super nucleophiles capable of displacing Br are the N₃⁻ and thiocyanate (SCN⁻) groups. Using compositions and methods described herein, the reaction sequence B becomes possible due to the particular reactivity of the C—Cl bond because of the enhanced reactivity of the C—Cl bond due to the presence of the electron withdrawing group (CO) in the alpha position.

Over the past decade, there has been a tremendous need for self-cleaning surfaces that was exacerbated following the advent of the COVID-19 pandemic. Indeed, transient solutions such as disinfecting wipes are labor-intensive, costly and not sustainable in the long term. In order to address the shortcomings of existing temporary solutions, numerous surface coating or grafting strategies were developed to confer long-lasting or near-permanent antimicrobial properties to environmental surfaces.

While metal-based coatings have been shown to be effective, they raise concerns of durability, toxicity and sustainability. In contrast, quaternary ammonium compounds (QAC) have long been known as potent and stable antimicrobial products when covalently grafted on surfaces. In the category of QAC, polymers were shown to be superior to monomers (classically Cis quaternized alkyl chain such as 3-(trihydroxysilyl) propyldimethyloctadecyl ammonium chloride). The latter class of molecules was described in the early 1970s and sold by Dow-Corning from the late 1970s. The main reason for the low effectiveness of quaternary ammonium monomers is likely due to a surface charge density difference between monomers and polymers.

Indeed, quaternary ammonium polymers can be turned into high-density QAC, exceeding 10¹⁵ charges/cm², which is the most commonly described threshold to achieve biocidal activity of surfaces. These compounds kill bacteria, viruses, and fungi even as a monolayer, provided that the charge density threshold is reached. This is not the case with simple quaternary alkyl-ammonium compounds.

Once a surface is covalently treated with biocidal polymers and/or compounds, it is common not to be able to visualize the polymer layer since these layers are invisible to the naked eye. This raises concerns of quality control despite the growing availability of portable X-ray fluorescence (XRF), which is expensive and does not easily provide data regarding the homogeneity of the surface treatment. It would therefore be highly desirable to use a graftable biocidal polymer that is detectable using UV light. Another advantage is that UV light has become an accessible, low-cost and easy to handle technology. Moreover, in the absence of UV light, the treated surfaces can remain invisible to the naked eye, providing aesthetic advantages. However, at any time, it would be possible to reveal the presence and distribution of the polymers and/or compound on the target surface using UV light.

In one aspect, the disclosure provides a series of novel biocidal polymers, compounds, and mixtures thereof that comprise detectable moieties which permit the visualization of the polymers and compounds. In a non-limiting example, these detectable polymers and compounds can be grafted onto a surface to produce a coating that can be visualized using methods known in the art, such as UV light. The ability to visualize the polymers and compounds following their deposition on a surface allows for confirmation that the coating is indeed present on the surface, and for the determination of their distribution on the surface.

In other embodiments, these detectable polymers and compounds can be used in biocidal and antimicrobial compositions that are useful to combat healthcare-acquired infections (HAI) and virtually any type of environmental surface treatment. The detectable biocidal polymers and compounds of the disclosure can be used to contain and control the spread of infectious pathogens in a variety of health and industrial applications.

In some embodiments, compositions and polymers comprising the moieties of the disclosure can be visualized, are graftable, and provide bonds with improved stability and less sensitivity to hydrolysis than other moieties.

In one aspect of the disclosure, the polymers are prepared by covalently linking the chemical moieties to the polymer to produce polymers that can be easily grafted onto a variety of surfaces, including metal and wood. In some embodiments, the polymers are prepared in a one-pot synthesis. In another aspect of the disclosure, compounds described herein can be easily grafted onto surfaces. When grafted, functional groups on the chemical moieties and compounds of the disclosure can form covalent bonds with functional groups on the surface.

In one aspect, the disclosure provides polymers and compounds comprising one or more detectable moieties.

Any detectable moiety is contemplated by the present invention, as would be understood by one of ordinary skill in the art. As used herein, the detectable moiety is a chemical group, structure or compound that possesses a specifically identifiable physical property which can be distinguished from the physical properties of other polymers and/or compounds and/or moieties. Fluorescence, phosphorescence and luminescence including electroluminescence, chemiluminescence and bioluminescence are all detectable physical properties not found in most substances, but known to occur or to be inducible in others. For example, reactive derivatives of dansyl, coumarins, rhodamine and fluorescein are all inherently fluorescent when excited with light of a specific wavelength and can be specifically bound or attached to other substances. Coumarin has a high fluorescent quantum yield, higher than even a dansyl moiety, and facilitates detection where very low levels of target that are being sought. In some embodiments, certain detectable moieties are combined to facilitate detection or isolation.

In some embodiments, the detectable moiety is one or more selected from a fluorescent moiety, a phosphorescent moiety, and a luminescent moiety. In some embodiments, the fluorescent moieties is selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety. Non-limiting examples of fluorescent moieties include pyrene, sulfonated pyrene, sulfonated coumarin, sulfonated carbocyanine, sulfonated xanthene, anthracene, naphthalene, acridine, stilbene, indole, isoindole, indolizine, benzoindole, oxazole or benzoxazole, thiazole or benzothiazole, 4-amino-7-nitrobenzo-2-oxa-1,3-diazole (NBD), carbocyanine, carbostyryl, porphyrin, salicylate, anthranilate, azulene, perylene, pyridine, quinoline, isoquinoline, chromene, borapolyazaindacene, xanthene, fluorescein, rosamine, b Damine, rhodamine, benzofluorescein or dibenzofluorescein, seminaphthofluorescein, naphthofluorescein, bimane, oxazine or benzoxazine, carbazine, phenalenone, coumarin, benzofuran, benzophenalenone) and derivatives thereof. As used herein, oxazines include resorufin, aminooxazinone, diaminooxazine, and benzo-substituted analogs thereof. Non-limiting examples of phosphorescent dyes include metal-ligand complexes such as tris(2-phenypyridine)iridium [Ir(ppy)3]; 2,3,7,8,12,13,17,18-octaethyl-21H,23H-porphyrin platinum (II) [PtOEP] and organic dyes such as eosin (2″,4″,5″,7″-tetrabromofluorescein), 2,2″-bipyridine and erythrosin (2″,4″,5″,7″-tetraiodofluorescein). Non-limiting examples of luminescent moieties include luciferin moieties. In some embodiments, the detectable moiety comprises a coumarin moiety.

In one aspect, the disclosure provides a detectable moiety of formula (I):

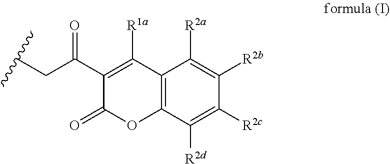

wherein in formula (I):
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, in formula (I), R^{1a} is hydrogen.

In some embodiments, in formula (I), R^{2a} is hydrogen. In some embodiments, in formula (I), R^{2b} is hydrogen. In some embodiments, in formula (I), R^{2c} is hydrogen. In some embodiments, in formula (I), R^{2d} is hydrogen. In some embodiments, in formula (I), each R^{2a}, R^{2b}, R^{2c}, and R^{2d} is hydrogen. In some embodiments, in formula (I), each R^{1a}, R^{2a}, R^{2b}, R^{2c}, and R^{2d} is hydrogen.

In some embodiments, the polymer comprises one or more detectable moieties of formula (I). In some embodiments, the polymer comprises a polyvinylpyridine (PVP) polymer comprising one or more detectable moieties of formula (I). In some embodiments, the polymer comprises a polyvinylbenzylchloride (PVBC) polymer comprising one or more detectable moieties of formula (I). In some embodiments, the polymer comprises a polyethyleneimine (PEI) polymer, optionally an alkyl-PEI polymer, optionally a methyl PEI polymer, comprising one or more detectable moieties of formula (I). In some embodiments, the compound comprises one or more detectable moieties of formula (I).

In one aspect, the disclosure provides a detectable moiety of formula (10):

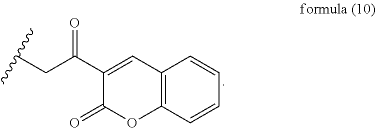 [SMILES: *CC(=O)c1cc2ccccc2oc1=O]

In some embodiments, the polymer comprises one or more detectable moieties of formula (10). In some embodiments, the polymer comprises a polyvinylpyridine (PVP) polymer comprising one or more detectable moieties of formula (10). In some embodiments, the polymer comprises a polyvinylbenzylchloride (PVBC) polymer comprising one or more detectable moieties of formula (I). In some embodiments, the polymer comprises a polyethyleneimine (PEI) polymer, optionally an alkyl-PEI polymer, optionally a methyl PEI polymer, comprising one or more detectable moieties of formula (10). In some embodiments, the compound comprises one or more detectable moieties of formula (I).

In one aspect, the disclosure provides a detectable moiety of formula (11):

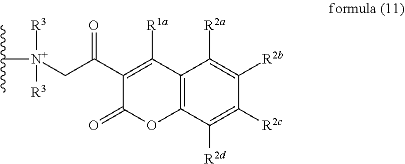

wherein in formula (11):
R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; and each R³ is independently optionally substituted alkyl.

In some embodiments, in formula (11), R^{1a} is hydrogen.

In some embodiments, in formula (11), R^{2a} is hydrogen. In some embodiments, in formula (11), R^{2b} is hydrogen. In some embodiments, in formula (11), R^{2c} is hydrogen. In some embodiments, in formula (11), R^{2d} is hydrogen. In some embodiments, in formula (11), each R^{2a}, R^{2b}, R^{2c}, and R^{2d} is hydrogen.

In some embodiments, in formula (11), each R^{1a}, R^{2a}, R^{2b}, R^{2c}, and R^{2d} is hydrogen, and each R³ is C₁-C₃, optionally each R³ is ethyl.

In some embodiments, the polymer comprises one or more detectable moieties of formula (11). In some embodiments, the polymer comprises a polyvinylpyridine (PVP) polymer comprising one or more detectable moieties of formula (11). In some embodiments, the polymer comprises a polyvinylbenzylchloride (PVBC) polymer comprising one or more detectable moieties of formula (11). In some embodiments, the polymer comprises a polyethyleneimine (PEI) polymer, optionally an alkyl-PEI polymer, optionally a methyl PEI polymer, comprising one or more detectable moieties of formula (11). In some embodiments, the compound comprises one or more detectable moieties of formula (11).

In one aspect, the disclosure provides a detectable moiety of formula (12):

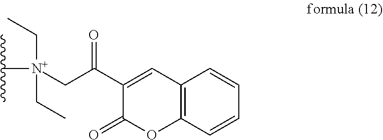 [SMILES: *[N+](CC)(CC)CC(=O)c1cc2ccccc2oc1=O]

In some embodiments, the polymer comprises one or more detectable moieties of formula (12). In some embodiments, the polymer is a polyvinylpyridine (PVP) polymer comprising one or more detectable moieties of formula (12). In some embodiments, the polymer comprises a polyvinylbenzylchloride (PVBC) polymer comprising one or more detectable moieties of formula (12). In some embodiments, the polymer is a polyethyleneimine (PEI) polymer, optionally an alkyl-PEI polymer, optionally a methyl PEI polymer, comprising one or more detectable moieties of formula (12). In some embodiments, the compound comprises one or more detectable moieties of formula (12).

In one aspect, the disclosure provides a detectable moiety of formula (II):

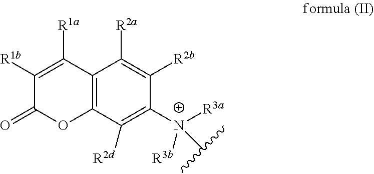

wherein in formula (II):
each R^{1a} and R^{1b} is independently optionally selected from hydrogen and alkyl; each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R^{3a} and R^{3a} is independently optionally substituted alkyl

In some embodiments, R^{1a} is hydrogen. In some embodiments, R^{1a} is C₁-C₃ alkyl. In some embodiments, R^{1a} is methyl. In some embodiments, R^{1b} is hydrogen. In some embodiments, each R^{1a} and R^{1b} is hydrogen. In some embodiments, R^{1a} is methyl and R^{1b} is hydrogen.

In some embodiments, R^{2a} is hydrogen. In some embodiments, R^{2b} is hydrogen. In some embodiments, R², is hydrogen. In some embodiments, R^{2c} is dialkylamino. In some embodiments, R², is diethylamino. In some embodiments, R^{2d} is hydrogen. In some embodiments, each R^{2a}, R^{2b}, R^{2c}, and R^{2d} is hydrogen. In some embodiments, each R^{2a}, R^{2b}, and R^{2d} is hydrogen and R^{2e} is diethylamino. In some embodiments, R^{3a} and R^{3b} are each independently optionally substituted C₁-C₃ alkyl. In some embodiments, R^{3a} is ethyl. In some embodiments, R^{3b} is ethyl. In some embodiments, R^{3a} and R^{3b} are each ethyl.

In some embodiments, in formula (II), R^{1a} is C₁-C₃ alkyl, optionally R^{1a} is methyl, each R^{1b} R^{2a}, R^{2b}, and R^{2d} is hydrogen, and each R^{3a} and R^{3b} is C₁-C₃ alkyl, optionally each R^{3a} and R^{3b} is ethyl.

In some embodiments, the polymer comprises one or more detectable moieties of formula (II). In some embodiments, the polymer comprises a polyvinylpyridine (PVP) polymer comprising one or more detectable moieties of formula (II). In some embodiments, the polymer comprises a polyvinylbenzylchloride (PVBC) polymer comprising one or more detectable moieties of formula (II). In some embodiments, the polymer comprises a polyethyleneimine (PEI) polymer, optionally an alkyl-PEI polymer, optionally a methyl PEI polymer, comprising one or more detectable moieties of formula (II). In some embodiments, the compound comprises one or more detectable moieties of formula (II).

In one aspect, the disclosure provides a detectable moiety of formula (20):

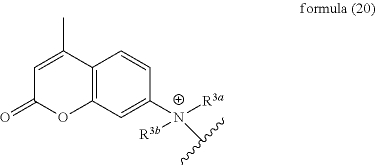

wherein in formula (20), R^{3a} and R^{3b} are each independently optionally substituted alkyl.

In some embodiments, R^{3a} and R^{3b} are each independently optionally substituted alkyl. In some embodiments, R^{3a} and R^{3b} are each independently optionally substituted C₁-C₃ alkyl. In some embodiments, R^{3a} and R^{3b} are each ethyl.

In some embodiments, the polymer comprises one or more detectable moieties of formula (20). In some embodiments, the polymer comprises one or more detectable moieties of formula (20). In some embodiments, the polymer comprises a polyvinylpyridine (PVP) polymer comprising one or more detectable moieties of formula (20). In some embodiments, the polymer comprises a polyvinylbenzylchloride (PVBC) polymer comprising one or more detectable moieties of formula (20). In some embodiments, the polymer comprises a polyethyleneimine (PEI) polymer, optionally an alkyl-PEI polymer, optionally a methyl PEI polymer, comprising one or more detectable moieties of formula (20). In some embodiments, the compound comprises one or more detectable moieties of formula (20).

In one aspect, the disclosure provides a detectable moiety of formula (21):

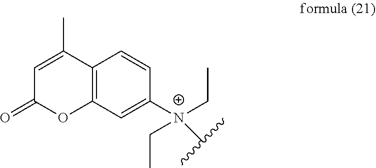 [SMILES: *[N+](CC)(CC)c1ccc2c(C)cc(=O)oc2c1]

In some embodiments, the polymer comprises one or more detectable moieties of formula (21). In some embodiments, the polymer comprises one or more detectable moieties of formula (21). In some embodiments, the polymer comprises a polyvinylpyridine (PVP) polymer comprising one or more detectable moieties of formula (21). In some embodiments, the polymer comprises a polyvinylbenzylchloride (PVBC) polymer comprising one or more detectable moieties of formula (21). In some embodiments, the polymer comprises a polyethyleneimine (PEI) polymer, optionally an alkyl-PEI polymer, optionally a methyl PEI polymer, comprising one or more detectable moieties of formula (21). In some embodiments, the compound comprises one or more detectable moieties of formula (21).

In one aspect, the disclosure provides a detectable moiety of formula (VII):

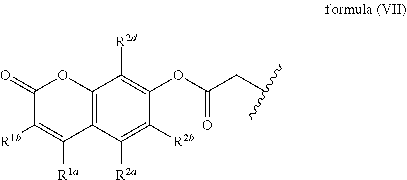

wherein in formula (VII):
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, in formula (VII), R^{1a} is hydrogen. In some embodiments, in formula (VII), R^{1b} is hydrogen.

In some embodiments, in formula (VII), R^{2a} is hydrogen. In some embodiments, in formula (VII), R^{2b} is hydrogen. In some embodiments, in formula (VII), R^{2d} is hydrogen. In some embodiments, in formula (VII), each R^{2a}, R^{2b}, R²¹, and R^{2d} is hydrogen. In some embodiments, in formula (VII), each R^{1a}, R^{1b}, R^{2a}, R^{2b}, and R^{2d} is hydrogen.

In some embodiments, the polymer comprises one or more detectable moieties of formula (VII). In some embodiments, the polymer comprises a polyvinylpyridine (PVP) polymer comprising one or more detectable moieties of formula (VII). In some embodiments, the polymer comprises a polyvinylbenzylchloride (PVBC) polymer comprising one or more detectable moieties of formula (VII). In some embodiments, the polymer comprises a polyethyleneimine (PEI) polymer, optionally an alkyl-PEI polymer, optionally a methyl PEI polymer, comprising one or more detectable moieties of formula (VII). In some embodiments, the compound comprises one or more detectable moieties of formula (VII).

In one aspect, the disclosure provides a detectable moiety of formula (70):

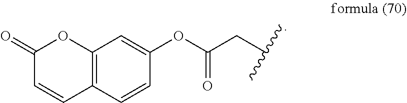 [SMILES: *CC(=O)Oc1ccc2ccc(=O)oc2c1]

In some embodiments, the polymer comprises one or more detectable moieties of formula (70). In some embodiments, the polymer comprises a polyvinylpyridine (PVP) polymer comprising one or more detectable moieties of formula (70). In some embodiments, the polymer comprises a polyvinylbenzylchloride (PVBC) polymer comprising one or more detectable moieties of formula (70). In some embodiments, the polymer comprises a polyethyleneimine (PEI) polymer, optionally an alkyl-PEI polymer, optionally a methyl PEI polymer, comprising one or more detectable moieties of formula (70). In some embodiments, the compound comprises one or more detectable moieties of formula (70).

In one aspect, the disclosure provides polymers that comprise a detectable moiety of the disclosure.

In one aspect, the disclosure provides a detectable moiety of formula (Iaa):

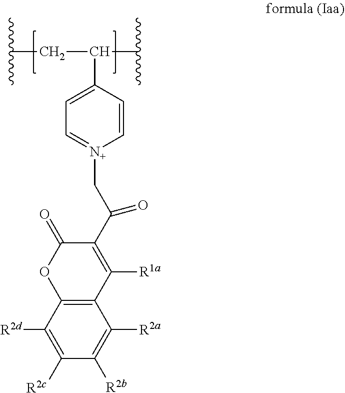

wherein in formula (Iaa):
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, in formula (Iaa), R^{1a} is hydrogen. In some embodiments, in formula (Iaa), R^{2a} is hydrogen. In some embodiments, in formula (Iaa), R^{2b} is hydrogen. In some embodiments, in formula (Iaa), R^{2c} is hydrogen. In some embodiments, in formula (Iaa), R^{2d} is hydrogen. In some embodiments, in formula (Iaa), each R^{2a}, R^{2b}, R^{2c}, and R^{2d} is hydrogen.

In one aspect, the disclosure provides a polymer comprising a detectable moiety of formula (Iaa). In some embodiments, the polymer comprises polyvinylpyridine (PVP). In some embodiments, the polymer comprises at least one moiety of formula (Ia).

In one aspect, the disclosure provides a detectable moiety of formula (Ia):

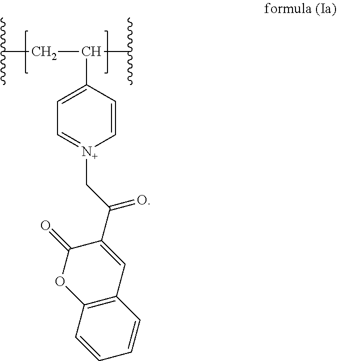

In one aspect, the disclosure provides a polymer comprising a detectable moiety of formula (Ia). In some embodiments, the polymer comprises polyvinylpyridine (PVP). In some embodiments, the polymer comprises at least one moiety of formula (Ia).

In one aspect, the disclosure provides a detectable moiety of formula (VIIa):

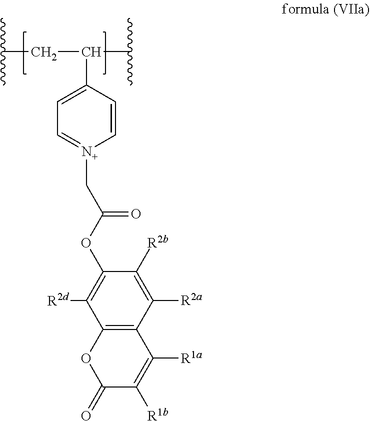

wherein in formula (VIIa):
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, in formula (VIIa), R^{1a} is hydrogen. In some embodiments, in formula (VIIa), R^{1b} is hydrogen.

In some embodiments, in formula (VIIa), R^{2a} is hydrogen. In some embodiments, in formula (VIIa), R^{2b} is hydrogen. In some embodiments, in formula (VIIa), R^{2d} is hydrogen. In some embodiments, in formula (VIIa), each R^{2a}, R^{2b}, R^{2c}, and R^{2d} is hydrogen. In some embodiments, in formula (VIIa), each R^{1a}, R^{1b}, R^{2a}, R^{2b}, and R^{2d} is hydrogen.

In one aspect, the disclosure provides a polymer comprising a detectable moiety of formula (VIIa). In some embodiments, the polymer comprises polyvinylpyridine (PVP). In some embodiments, the polymer comprises at least one moiety of formula (VIIa).

In one aspect, the disclosure provides a detectable moiety of formula (VIIb):

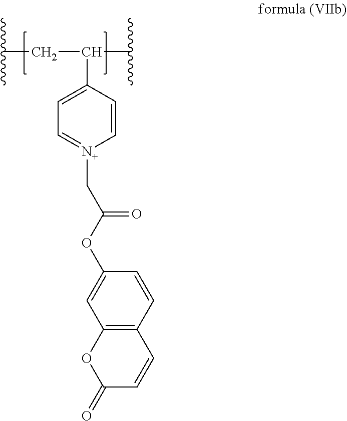

In one aspect, the disclosure provides a polymer comprising a detectable moiety of formula (VIIb). In some embodiments, the polymer comprises polyvinylpyridine (PVP). In some embodiments, the polymer comprises at least one moiety of formula (VIIb).

In one aspect, the polymer comprises at least one moiety of formula (Ia), at least one moiety of formula (3), and at least one moiety of formula (4):

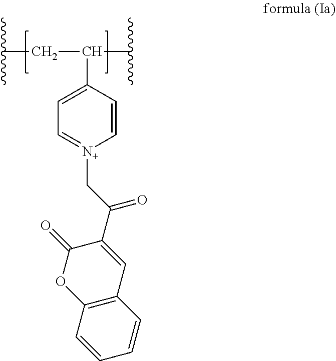

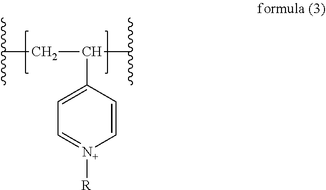

wherein in formula (3):
R is C₄-C18 alkyl or benzyl;

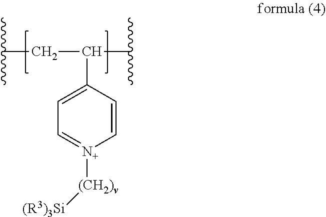

wherein in formula (4):
each R³ is independently optionally substituted alkoxy; and
v is an integer from 3 to 10.

In one aspect, the polymer comprises at least one moiety of formula (Iaa), at least one moiety of formula (3), and at least one moiety of formula (4); wherein in formula (3), R is C₄-C₁₈ alkyl or benzyl; and wherein in formula (4), each R³ is independently optionally substituted alkoxy; and v is an integer from 3 to 10.

In one aspect, the polymer comprises at least one moiety of formula (VIIa), at least one moiety of formula (3), and at least one moiety of formula (4); wherein each R^{1a} and R^{1b} is independently optionally selected from hydrogen and alkyl; and each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; wherein in formula (3), R is C₄-C18 alkyl or benzyl; wherein in formula (4), each R³ is independently optionally substituted alkoxy; and v is an integer from 3 to 10.

In some embodiments, in formula (Iaa), R^{1a} is hydrogen. In some embodiments, in formula (Iaa), R^{2a} is hydrogen. In some embodiments, in formula (Iaa), R^{2b} is hydrogen. In some embodiments, in formula (Iaa), R² is hydrogen. In some embodiments, in formula (Iaa), R^{2d} is hydrogen. In some embodiments, in formula (Iaa), each R^{1a}, R^{2a}, R^{2b}, R^{2c}, and R^{2d} is hydrogen.

In some embodiments, in formula (VIIa), R^{1a} is hydrogen. In some embodiments, in formula (VIIa), R^{1b} is hydrogen. In some embodiments, in formula (VIIa), R^{2a} is hydrogen. In some embodiments, in formula (VIIa), R^{2b} is hydrogen. In some embodiments, in formula (VIIa), R^{2d} is hydrogen. In some embodiments, in formula (VIIa), each R^{2a}, R^{2b}, R^{2c}, and R^{2d} is hydrogen. In some embodiments, in formula (VIIa), each R^{1a}, R^{1b}, R^{2a}, R^{2b}, and R^{2d} is hydrogen.

In some embodiments, in formula (3), R is C₄-C₁₀ alkyl, C₄-C₈ alkyl, or C₄-C₆ alkyl.

In some embodiments, R is butyl.

In one aspect, the polymer comprises at least one moiety of formula (VIIb), at least one moiety of formula (3), and at least one moiety of formula (4); wherein in formula (3), R is C₄-C₁₈ alkyl or benzyl; and wherein in formula (4), each R³ is independently optionally substituted alkoxy; and v is an integer from 3 to 10.

In some embodiments, in formula (4), v is an integer from 3 to 5. In some embodiments, in formula (4), v is 3. In some embodiments, in formula (4), each R³ is C₁-C₃ alkoxy. In some embodiments, each R³ is methoxy. In some embodiments, in formula (IV), v is 3 and each R³ is methoxy. In some embodiments, in formula (4), v is an integer from 3 to 5, optionally v is 3, and each R³ is C₁-C₃ alkoxy, optionally R³ is methoxy.

In some embodiments, the polymer comprises at least one moiety of formula (Ia), at least one moiety of formula (5), and at least one moiety of formula (6):

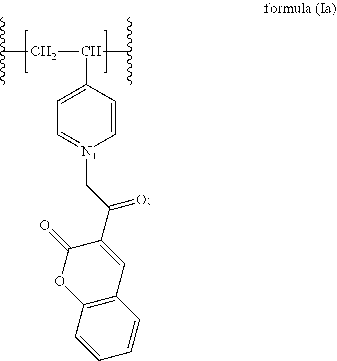

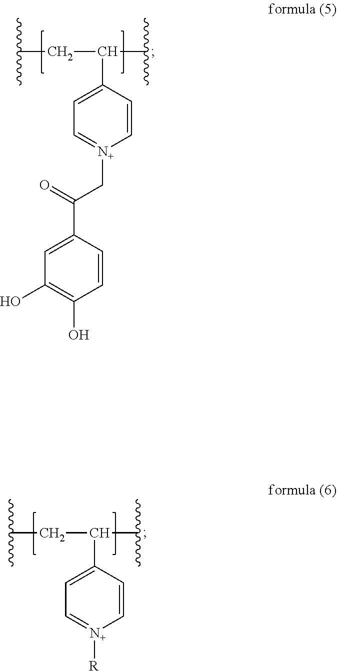

wherein in formula (6):
R is C₄-C18 alkyl or benzyl.

In some embodiments, the polymer comprises at least one moiety of formula (Iaa), at least one moiety of formula (5), and at least one moiety of formula (6), wherein in formula (6), R is C₄-C18 alkyl or benzyl.

In some embodiments, in formula (Iaa), R^{1a} is hydrogen. In some embodiments, in formula (Iaa), R^{2a} is hydrogen. In some embodiments, in formula (Iaa), R^{2b} is hydrogen. In some embodiments, in formula (Iaa), R² is hydrogen. In some embodiments, in formula (Iaa), R^{2d} is hydrogen. In some embodiments, in formula (Iaa), each R^{1a}, R^{2a}, R^{2b}, R^{2c}, and R^{2d} is hydrogen.

In one aspect, the polymer comprises at least one moiety of formula (VIIa), at least one moiety of formula (5), and at least one moiety of formula (6); wherein in formula (VIIa), each R^{1a} and R^{1b} is independently optionally selected from hydrogen and alkyl; and each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; wherein in formula (6), R is C₄-C18 alkyl or benzyl.

In some embodiments, in formula (VIIa), R^{1a} is hydrogen. In some embodiments, in formula (VIIa), R^{1b} is hydrogen. In some embodiments, in formula (VIIa), R^{2a} is hydrogen. In some embodiments, in formula (VIIa), R^{2b} is hydrogen. In some embodiments, in formula (VIIa), R^{2d} is hydrogen. In some embodiments, in formula (VIIa), each R^{2a}, R^{2b}, R^{2c}, and R^{2d} is hydrogen. In some embodiments, in formula (VIIa), each R^{1a}, R^{1b}, R^{2a}, R^{2b}, and R^{2d} is hydrogen.

In some embodiments, the polymer comprises at least one moiety of formula (VIIb), at least one moiety of formula (5), and at least one moiety of formula (6), wherein in formula (6), R is C₄-C18 alkyl or benzyl.

In some embodiments, in formula (6), R is C₄-C₁₀ alkyl, C₄-C₈ alkyl, or C₄-C₆ alkyl.

In some embodiments, in formula (6), R is butyl.

In one aspect, the disclosure provides a detectable moiety of formula (Ibb):

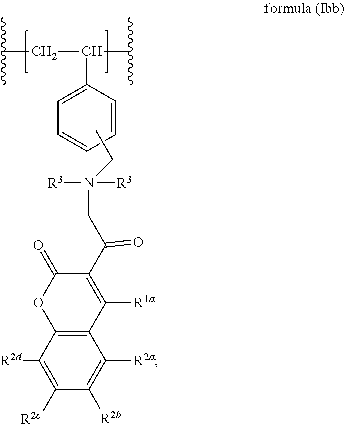

wherein in formula (Ibb):
R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkyl.

In one aspect, the disclosure provides a polymer comprising a detectable moiety of formula (Ibb). In some embodiments, the polymer comprises polyvinylbenzylchloride (PVBC). In some embodiments, the polymer comprises at least one moiety of formula (Ibb).

In some embodiments, in formula (Ibb), R^{1a} is hydrogen. In some embodiments, in formula (Ibb), R^{2a} is hydrogen. In some embodiments, in formula (Ibb), R^{2b} is hydrogen. In some embodiments, in formula (Ibb), R^{2c} is hydrogen. In some embodiments, in formula (Ibb), R^{2d} is hydrogen. In some embodiments, in formula (Ibb), each R^{1a}, R^{2a}, R^{2b}, R^{2c}, and R^{2d} is hydrogen.

In some embodiments, in formula (Ibb), each R³ is C₁-C₅ alkyl or C₁-C₃ alkyl. In some embodiments, in formula (Ibb), each R³ is ethyl. In some embodiments, in formula (Ibb), each R^{1a}, R^{2a}, R^{2b}, R^{2c}, and R^{2d} is hydrogen, and each R³ is C₁-C₃, optionally each R³ is ethyl.

In one aspect, the disclosure provides a detectable moiety of formula (Ib):

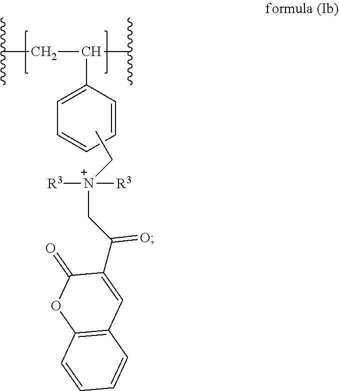

wherein in formula (Ib):
each R³ is independently optionally substituted alkyl.

In one aspect, the disclosure provides a polymer comprising a detectable moiety of formula (Ib). In some embodiments, the polymer comprises polyvinylbenzylchloride (PVBC). In some embodiments, the polymer comprises at least one moiety of formula (Ib).

In some embodiments, in formula (Ib), each R³ is C₁-C₅ alkyl or C₁-C₃ alkyl In some embodiments, in formula (Ib), each R³ is ethyl.

In some embodiments, the polymer comprises at least one moiety of formula (Ib) at least one moiety of formula (7), and at least one moiety of formula (8):

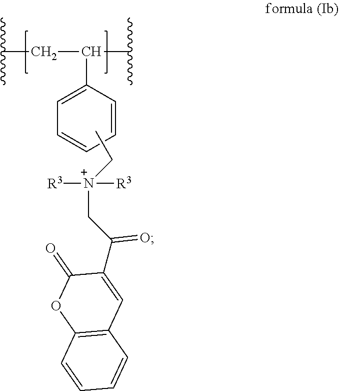

wherein in formula (Ib):
each R³ is independently optionally substituted alkyl;

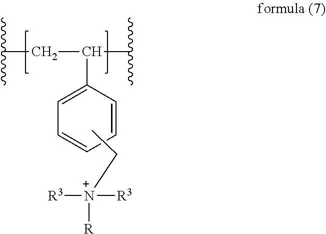

wherein in formula (7):
each R³ is independently optionally substituted alkyl; and
R is C₄-C18 alkyl or benzyl;

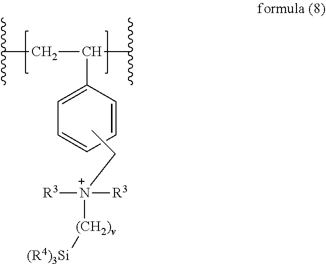

wherein in formula (8):
each R³ is independently optionally substituted alkyl;
each R⁴ is independently optionally substituted alkoxy; and
v is an integer from 3 to 10.

In some embodiments, the polymer comprises at least one moiety of formula (Ibb), at least one moiety of formula (7), each R³ is independently optionally substituted alkyl; and R is C₄-C18 alkyl or benzyl; and at least one moiety of formula (8), wherein in formula (8), each R³ is independently optionally substituted alkyl; each R⁴ is independently optionally substituted alkoxy, and v is an integer from 3 to 10.

In some embodiments, in formula (Ib), each R³ is C₁-C₅ alkyl or C₁-C₃ alkyl. In some embodiments, in formula (Ib), each R³ is ethyl. In some embodiments, in formula (Ibb), each R³ is C₁-C₅ alkyl. In some embodiments, in formula (Ibb), each R³ is ethyl.

In some embodiments, in formula (Ibb), R^{1a} is hydrogen. In some embodiments, in formula (Ibb), R^{2a} is hydrogen. In some embodiments, in formula (Ibb), R^{2b} is hydrogen. In some embodiments, in formula (Ibb), R^{2c} is hydrogen. In some embodiments, in formula (Ibb), R^{2d} is hydrogen. In some embodiments, in formula (Ibb), each R^{1a}, R^{2a}, R^{2b}, R^{2c}, and R^{2d} is hydrogen.

In some embodiments, in formula (Ibb), each R^{1a}, R^{2a}, R^{2b}, R^{2c}, and R^{2d} is hydrogen, and each R³ is C₁-C₃, optionally each R³ is ethyl.

In some embodiments, in formula (Ib), each R³ is C₁-C₅ alkyl. In some embodiments, in formula (Ib), each R³ is ethyl. In some embodiments, in formula (Ibb), each R³ is C₁-C₅ alkyl. In some embodiments, in formula (Ibb), each R³ is ethyl.

In some embodiments, in formula (7), R is C₄-C₁₀ alkyl, C₄-C₈ alkyl, or C₄-C₆ alkyl.

In some embodiments, in formula (7), R is butyl. In some embodiments, in formula (7), each R³ is C₁-C₅ alkyl. In some embodiments, in formula (7), each R³ is methyl. In some embodiments, in formula (7), R is C₄-C₁₀ alkyl, C₄-C₈ alkyl, or C₄-C₆ alkyl, optionally R is butyl, and R³ is C₁-C₅ alkyl, optionally each R³ is methyl.

In some embodiments, in formula (8), v is an integer from 3 to 5. In some embodiments, in formula (8), v is 3. In some embodiments, in formula (8), each R⁴ is C₁-C₃ alkoxy. In some embodiments, in formula (8), each R⁴ is methoxy. In some embodiments, in formula (8), v is 3 and each R⁴ is methoxy. In some embodiments, in formula (8), v is an integer from 3 to 5, optionally v is 3, and each R⁴ is C₁-C₃ alkoxy, optionally R⁴ is methoxy.

In some embodiments, the polymer comprises at least one moiety of formula (IIaa) at least one moiety of formula (7), and at least one moiety of formula (8):

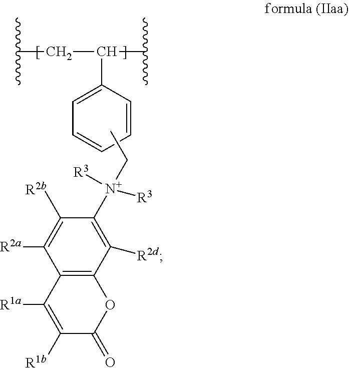

wherein in formula (IIaa):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkyl;

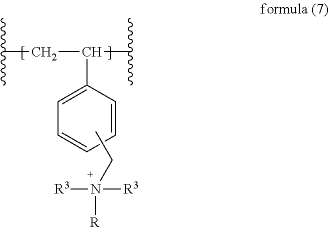

wherein in formula (7):
each R³ is independently optionally substituted alkyl; and
R is C₄-C18 alkyl or benzyl;

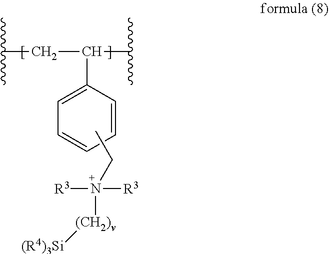

wherein in formula (8):
each R³ is independently optionally substituted alkyl;
each R⁴ is independently optionally substituted alkoxy; and
v is an integer from 3 to 10.

In some embodiments, the polymer comprises at least one moiety of formula (IIa) at least one moiety of formula (7), and at least one moiety of formula (8):

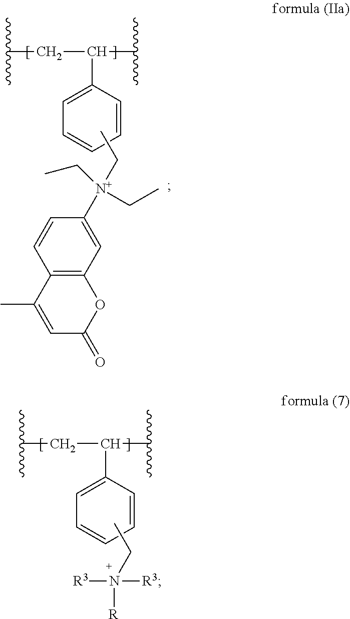

wherein in formula (7):
each R³ is independently optionally substituted alkyl; and
R is C₄-C18 alkyl or benzyl;

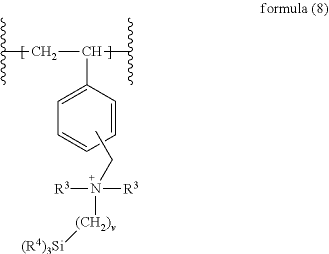

wherein in formula (8):
each R³ is independently optionally substituted alkyl;
each R⁴ is independently optionally substituted alkoxy; and
v is an integer from 3 to 10.

In some embodiments, in formula (IIaa), R^{1a} is hydrogen. In some embodiments, in formula (IIaa), R^{1a} is C₁-C₃ alkyl. In some embodiments, in formula (IIaa), R^{1a} is methyl. In some embodiments, in formula (IIaa), R^{1b} is hydrogen. In some embodiments, in formula (IIaa), R^{2a} is hydrogen. In some embodiments, in formula (IIaa), R^{2b} is hydrogen. In some embodiments, in formula (IIaa), R^{2d} is hydrogen. In some embodiments, in formula (Iaa), each R^{1a}, R^{1b} R^{2a}, R^{2b}, and R^{2d} is hydrogen. In some embodiments, in formula (IIaa), R^{1a} is methyl and each R^{1b} R^{2a}, R^{2b}, and R^{2d} is hydrogen. In some embodiments, in formula (IIaa), each R³ is C₁-C₃ alkyl. In some embodiments, in formula (IIaa), each R³ is ethyl. In some embodiments, in formula (IIaa), R^{1a} is C₁-C₃ alkyl, optionally R^{1a} is methyl, each R^{1b} R^{2a}, R^{2b}, and R^{2d} is hydrogen, and each R³ is C₁-C₃ alkyl, optionally each R³ is ethyl.

In some embodiments, the polymer comprises at least one moiety of formula (IIa) at least one moiety of formula (7), and at least one moiety of formula (13), wherein in formula (7), wherein in formula (7) each R³ is independently optionally substituted alkyl; and R is C₄-C18 alkyl or benzyl; and wherein in formula (13), each R³ is independently optionally substituted alkyl.

In some embodiments, in formula (7), R is C₄-C₁₂ alkyl, C₄-C₁₀ alkyl, C₄-C₈ alkyl, or C₄-C₆ alkyl. In some embodiments, in formula (7), R is butyl. In some embodiments, in formula (7), each R³ is C₁-C₅ alkyl. In some embodiments, in formula (7), each R³ is methyl. In some embodiments, in formula (7), R is C₄-C₁₂ alkyl, C₄-C₁₀ alkyl, C₄-C₈ alkyl, or C₄-C₆ alkyl, optionally R is butyl, and R³ is C₁-C₅ alkyl, optionally each R³ is methyl.

In some embodiments, in formula (8), v is an integer from 3 to 5. In some embodiments, in formula (8), v is 3. In some embodiments, in formula (8), each R⁴ is C₁-C₃ alkoxy. In some embodiments, in formula (8), each R⁴ is methoxy. In some embodiments, in formula (8), each R³ is C₁-C₅ alkyl or C₁-C₃ alkyl. In some embodiments, in formula (8), each R³ is methyl. In some embodiments, in formula (8), v is an integer from 3 to 5, optionally v is 3, each R³ is C₁-C₃ alkyl, optionally each R³ is methyl, and each R⁴ is C₁-C₃ alkoxy, optionally R⁴ is methoxy.

In some embodiments, in formula (7), each R³ is C₁-C₅ alkyl. In some embodiments, in formula (7), each R³ is methyl. In some embodiments, in formula (VII), R is butyl.

In some embodiments, in formula (8), each R³ is C₁-C₅ alkyl. In some embodiments, in formula (8), each R³ is methyl. In some embodiments, v is an integer from 3 to 5. In some embodiments, v is 3. In some embodiments, each R⁴ is C₁-C₅ alkoxy. In some embodiments, each R⁴ is methoxy. In some embodiments, in formula (8), each R³ is methyl, v is 3, and each R⁴ is methoxy.

In some embodiments, the polymer comprises at least one moiety of formula (IIaa) at least one moiety of formula (7), and at least one moiety of formula (13):

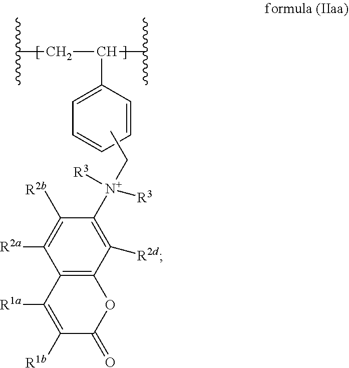

wherein in formula (IIaa):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkyl;

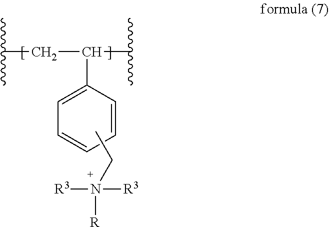

wherein in formula (7):
each R³ is independently optionally substituted alkyl; and
R is C₄-C18 alkyl or benzyl;

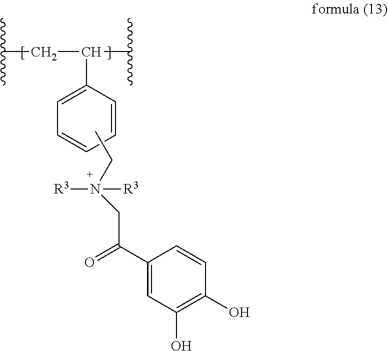

wherein in formula (13):
each R³ is independently optionally substituted alkyl.

In some embodiments, in formula (IIaa), R^{1a} is hydrogen. In some embodiments, in formula (IIaa), R^{1a} is C₁-C₃ alkyl. In some embodiments, in formula (IIaa), R^{1a} is methyl. In some embodiments, in formula (IIaa), R^{1b} is hydrogen. In some embodiments, in formula (IIaa), R^{2a} is hydrogen. In some embodiments, in formula (IIaa), R^{2b} is hydrogen. In some embodiments, in formula (IIaa), R^{2d} is hydrogen. In some embodiments, in formula (Iaa), each R^{1a}, R^{1b} R^{2a}, R^{2b}, and R^{2d} is hydrogen. In some embodiments, in formula (IIaa), R^{1a} is methyl and each R^{1b} R^{2a}, R^{2b}, and R^{2d} is hydrogen. In some embodiments, in formula (IIaa), each R³ is C₁-C₃ alkyl. In some embodiments, in formula (IIaa), each R³ is ethyl. In some embodiments, in formula (IIaa), R^{1a} is C₁-C₃ alkyl, optionally R^{1a} is methyl, each R^{1b} R^{2a}, R^{2b}, and R^{2d} is hydrogen, and each R³ is C₁-C₃ alkyl, optionally each R³ is ethyl.

In some embodiments, the polymer comprises at least one moiety of formula (IIa) at least one moiety of formula (7), and at least one moiety of formula (13), wherein in formula (7), each R³ is independently optionally substituted alkyl; and R is C₄-C18 alkyl or benzyl; and wherein in in formula (13), each R³ is independently optionally substituted alkyl.

In some embodiments, in formula (7), R is C₄-C₁₂ alkyl, C₄-C₁₀ alkyl, C₄-C₈ alkyl, or C₄-C₆ alkyl. In some embodiments, in formula (7), R is butyl. In some embodiments, in formula (7), each R³ is C₁-C₅ alkyl. In some embodiments, in formula (7), each R³ is methyl. In some embodiments, in formula (7), R is C₄-C₁₂ alkyl, C₄-C₁₀ alkyl, C₄-C₈ alkyl, or C₄-C₆ alkyl, optionally R is butyl, and R³ is C₁-C₅ alkyl, optionally each R³ is methyl.

In some embodiments, in formula (13), each R³ is C₄-C₁₂ alkyl, C₄-C₁₀ alkyl, C₄-C₈ alkyl, or C₄-C₆ alkyl. In some embodiments, in formula (13), each R³ is butyl. In some embodiments, in formula (13), each R³ is hexyl.

In some embodiments, in formula (7), each R³ is C₁-C₅ alkyl. In some embodiments, in formula (7), each R³ is methyl. In some embodiments, in formula (VII), R is butyl.

In some aspects, the disclosure provides a polyethyleneimine (PEI) polymer comprising at least one moiety of formula (IXa), formula (IXb), or formula (IXc):

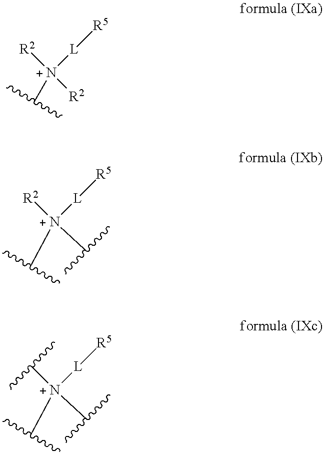

wherein in formula (IXa), formula (IXb), and formula (IXc):
L is a single bond or a linking group;
R⁵ is a detectable moiety; and
each R² is independently optionally substituted alkyl.

In some embodiments, in formula (IXa), (IXb), or (IXc), L is a single bond. In some embodiments, in formula (IXa), (IXb), or (IXc), L is a linking group. The linking group may be any organic moiety, as would be understood by one of ordinary skill in the art. In some embodiments, the linking group comprises an optionally substituted alkyl group and a carbonyl group. In some embodiments, in formula (IXa), (IXb), or (IXc), L is selected from

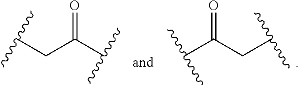 [SMILES: *CC(*)=O ; *CC(*)=O]

In some embodiments, the linking group comprises optionally substituted amino, optionally substituted heterocyclyl, carboxyl, or optionally substituted thiol. In some embodiments, the linking group comprises a primary amino group, a secondary amino group, or a tertiary amino group. In some embodiments, in formula (IXa), (IXb), or (IXc), the organic linker comprises dimethylamino, diethylamino, —C(O)O—, —S—, diethylcarboxylate, acetyl, optionally substituted triazole group, or optionally substituted tetrazole group.

In some embodiments, in formula (IXa), (IXb), or (IXc), each R² is C₁-C₅ alkyl or C₁-C₃ alkyl. In some embodiments, in formula (IXa), (IXb), or (IXc), each R² is independently methyl.

In some embodiments, the polymer is a polyethyleneimine (PEI) polymer comprising at least one moiety of formula (IXa1), formula (IXb1), or formula (IXc1):

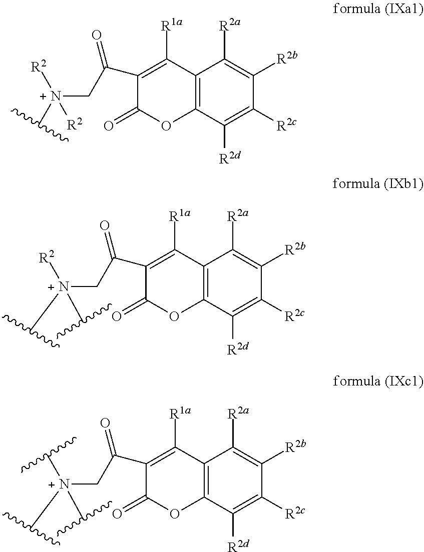

wherein in formula (IXa1), formula (IXb1), and formula (IXc1):
each R² is independently optionally substituted alkyl;
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, in formula (IXa1), (IXb1), or (IXc1), R^{1a} is hydrogen. In some embodiments, in formula (IXa1), (IXb1), or (IXc1), R^{2a} is hydrogen. In some embodiments, in formula (IXa1), (IXb1), or (IXc1), R^{2b} is hydrogen. In some embodiments, in formula (IXa1), (IXb1), or (IXc1), R^{2c} is hydrogen. In some embodiments, in formula (IXa1), (IXb1), or (IXc1), R^{2d} is hydrogen. In some embodiments, in formula (IXa1), (IXb1), or (IXc1), each R^{2a}, R^{2b}, R^{2e}, and R^{2d} is hydrogen.

In some embodiments, in formula (IXa1), (IXb1), or (IXc1), each R² is C₁-C₅ alkyl or C₁-C₃ alkyl. In some embodiments, in formula (IXa1), (IXb1), or (IXc1), each R² is independently methyl.

In some embodiments, the polymer is a polyethyleneimine (PEI) polymer comprising at least one moiety of formula (IXa2), formula (IXb2), or formula (IXc2):

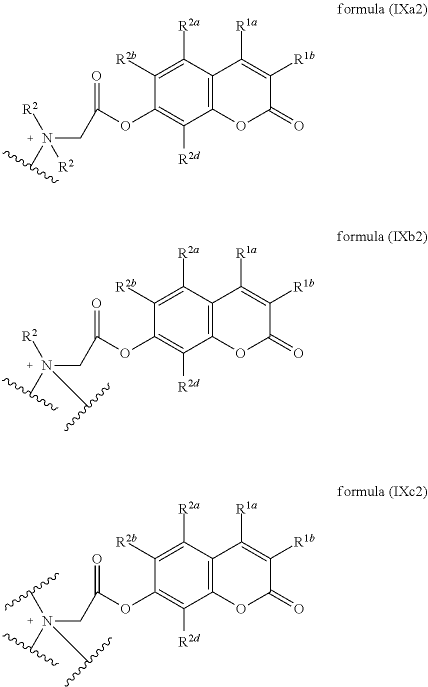

wherein in formula (IXa2), formula (IXb2), and formula (IXc2):
each R² is independently optionally substituted alkyl;
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, in formula (IXa1), (IXb1), or (IXc1), R^{1a} is hydrogen. In some embodiments, in formula (IXa1), (IXb1), or (IXc1), R^{1b} is hydrogen. In some embodiments, in formula (VII), R^{2a} is hydrogen. In some embodiments, in formula (IXa1), (IXb1), or (IXc1), R^{2b} is hydrogen. In some embodiments, in formula (IXa1), (IXb1), or (IXc1), R^{2d} is hydrogen. In some embodiments, in formula (IXa1), (IXb1), or (IXc1), each R^{2a}, R^{2b}, R^{2c}, and R^{2d} is hydrogen. In some embodiments, in formula (IXa1), (IXb1), or (IXc1), each R^{1a}, R^{1b}, R^{2a}, R^{2b} and R^{2d} is hydrogen.

In some embodiments, in formula (IXa2), (IXb2), or (IXc2), each R² is C₁-C₅ alkyl or C₁-C₃ alkyl. In some embodiments, in formula (IXa2), (IXb2), or (IXc2), each R² is independently methyl.

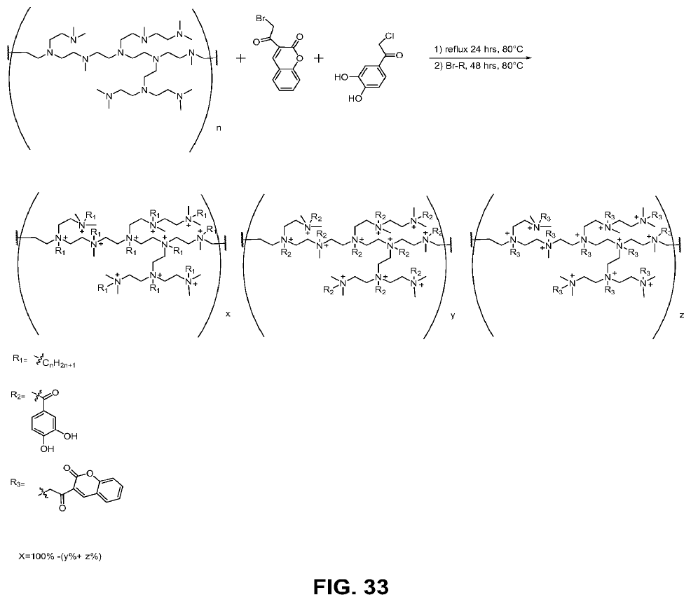

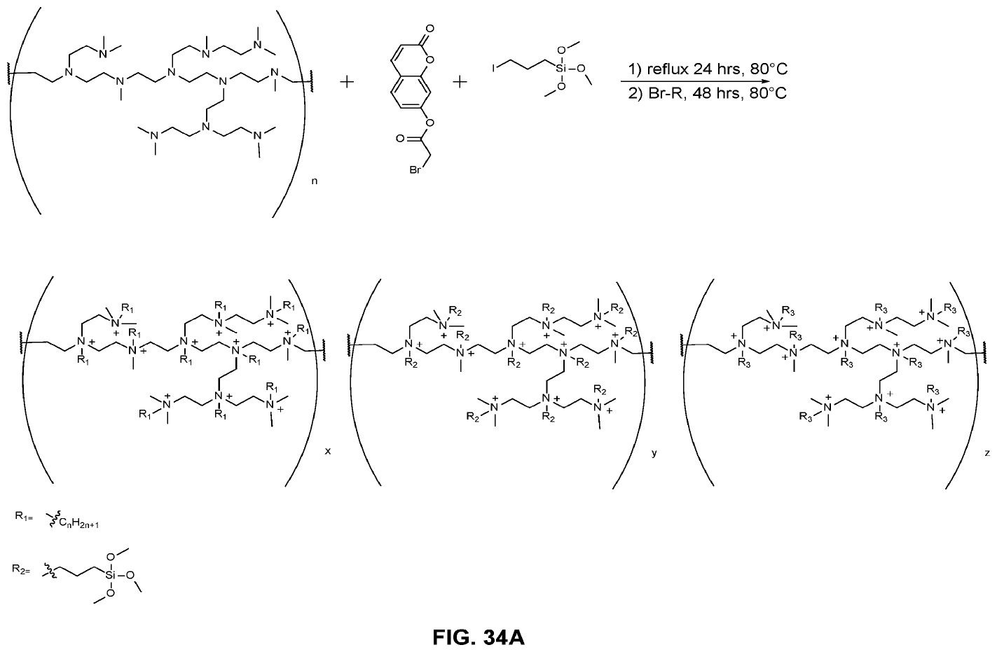

In some embodiments, the polymer is branched, hyperbranched or linear. In some embodiments, the PEI polymer is fully alkylated. In some embodiments, the PEI polymer is fully methylated. Non-limiting examples of fully methylated monomer are shown in FIGS. 33-36. As would be understood by one of ordinary skill in the art, in a non-limiting example a PEI polymer comprises primary and secondary nitrogen atoms, and when fully methylated, all primary and secondary nitrogens are converted to tertiary nitrogens comprising two and one methyl groups, respectively. In a non-limiting embodiment, the tertiary nitrogens can be quaternized following treatment with detectable moieties, resulting in mixture of moieties of formula (IXa) and/or moieties of formula (IXb) and/or moieties of formula (IXc). In a non-limiting example, remaining tertiary nitrogens can be converted into quaternary nitrogens comprising an optionally substituted C₄-C₂₂ alkyl group, as would be understood by one of ordinary skill in the art. In some embodiments, the PEI polymer further comprises at least one optionally substituted C₄-C₂₂ alkyl group. In some embodiments, the PEI polymer is partially quaternized. In some embodiments, the PEI polymer is fully quaternized. In some embodiments, the molar ratio of the total moieties of formula (IXa), (IXa1) and/or (IXa2) and moieties of formula (IXb), (IXb1) and/or (IXb2) to the optionally substituted C₄-C₂₂ alkyl group is about 0.05≤x≤0.5:(1-x). In some embodiments, the PEI polymer is fully quaternized. In some embodiments, the molar ratio of the total moieties of formula (IXa), (IXa1) and/or (IXa2) and moieties of formula (IXb), (IXb1) and/or (IXb2) and moieties of formula (IXc), (IXc1) and/or (IXc2) to the optionally substituted C₄-C₂₂ alkyl group is about 0.05≤x≤0.5:(1-x). In some embodiments, the PEI polymer consists of fully methylated monomers, wherein each monomer is fully quaternized and consists of moieties of formula (IXa), (IXa1) and/or (IXa2) and moieties of formula (IXb), (IXb1) and/or (IXb2), and optionally substituted C₄-C₂₂ alkyl groups. In some embodiments, the PEI polymer consists of fully methylated monomers, wherein each monomer is fully quaternized and consists of moieties of formula (IXa), (IXa1) and/or (IXa2) and moieties of formula (IXb), (IXb1) and/or (IXb2) and moieties of formula (IXc), (IXc1) and/or (IXc2), and optionally substituted C₄-C₂₂ alkyl groups. In some embodiments, the molar ratio of the total moieties of formula (IXa), (IXa1) and/or (IXa2) and moieties of formula (IXb), (IXb1) and/or (IXb2) to the optionally substituted C₄-C₂₂ alkyl groups is about 0.05≤x≤0.5:(1-x). In some embodiments, the molar ratio of the total moieties of formula (IXa), (IXa1) and/or (IXa2) and moieties of formula (IXb), (IXb1) and/or (IXb2) to the optionally substituted C₄-C₂₂ alkyl groups is about 0.1:0.9. In some embodiments, the molar ratio of the total moieties of formula (IXa), (IXa1) and/or (IXa2) and moieties of formula (IXb), (IXb1) and/or (IXb2) and moieties of formula (IXc), (IXc1) and/or (IXc2) to the optionally substituted C₄-C₂₂ alkyl groups is about 0.05≤x≤0.5:(1-x). In some embodiments, the molar ratio of the total moieties of formula (IXa), (IXa1) and/or (IXa2) and moieties of formula (IXb), (IXb1) and/or (IXb2) and moieties of formula (IXc), (IXc1) and/or (IXc2) to the optionally substituted C₄-C₂₂ alkyl groups is about 0.1:0.9.

In some embodiments, the PEI polymer comprises one or more moieties of formula (IXa), (IXa1) and/or (IXa2) and/or one or more moieties of formula (IXb), (IXb1) and/or (IXb2), and/or one or more moieties of formula (IXc), (IXc1) and/or (IXc2); and optionally substituted C₄-C₂₂ alkyl groups, and one or more moieties of

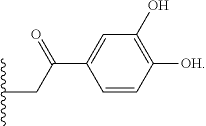 [SMILES: *CC(=O)c1ccc(O)c(O)c1]

In some embodiments, the molar ratio of the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of

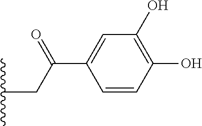 [SMILES: *CC(=O)c1ccc(O)c(O)c1]

groups is x:y:z, wherein about 0.05≤x≤0.4:0.2≤y≤0.9:0.05≤z≤0.4. In some embodiments, the molar ratio of the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of

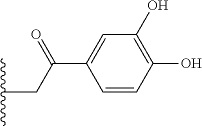 [SMILES: *CC(=O)c1ccc(O)c(O)c1]

groups is x:y:z, wherein about 0.08≤x≤0.12:0.76≤y≤0.84:0.08≤z≤0.12. In some embodiments, the molar ratio of the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of

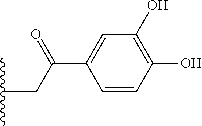 [SMILES: *CC(=O)c1ccc(O)c(O)c1]

groups is about 0.1:0.8:0.1. In some embodiments, the alkyl group is a C₁₀ group. In some embodiments, the alkyl group is a C₆ alkyl group. In some embodiments, the alkyl group is a C₄ alkyl group.

In some embodiments, the PEI polymer comprises one or more moieties of formula (IXa), (IXa1) and/or (IXa2) and/or one or more moieties of formula (IXb), (IXb1) and/or (IXb2), and/or one or more moieties of formula (IXc), (IXc1) and/or (IXc2); and optionally substituted C₄-C₂₂ alkyl groups, and one or more moieties of formula (9):

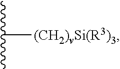

optionally

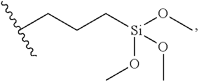

wherein each R³ is independently optionally substituted alkoxy; and v is an integer from 3 to 10. In some embodiments, the molar ratio of the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of moieties of formula (9) is x:y:z, wherein about 0.05≤x≤0.4:0.2≤y≤0.9:0.05≤z≤0.4. In some embodiments, the molar ratio of the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of moieties of formula (9) is x:y:z, wherein about 0.08≤x≤0.12:0.76≤y≤0.84:0.08≤z≤0.12. In some embodiments, the molar ratio of the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of moieties of formula (9) is about 0.1:0.8:0.1. In some embodiments, the alkyl group is a C₁₀ group. In some embodiments, the alkyl group is a C₆ alkyl group. In some embodiments, the alkyl group is a C₄ alkyl group.

In another aspect, the disclosure describes a PEI polymer comprising at least one moiety of formula (IXe), or substructures thereof:

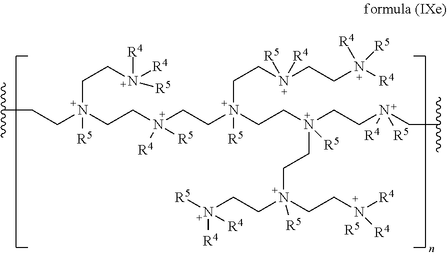

wherein in formula (IXe):
each R⁴ is independently optionally substituted alkyl; and
each R⁵ is independently optionally substituted alkyl, a moiety of formula (I):

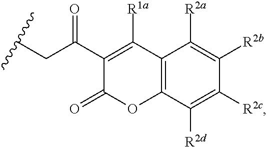

or a catechol moiety of formula (11):

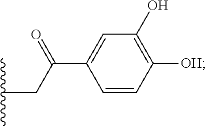 [SMILES: *CC(=O)c1ccc(O)c(O)c1]

R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl;
   with the proviso that at least one R⁵ is a moiety of formula (I):

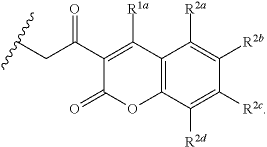

In some embodiments, in formula (IXe), each R⁴ is independently C₁-C₃ alkyl. In some embodiments, in formula (IXe), each R⁴ is methyl. In some embodiments, in formula (IXe), 1, 2, 3, 4, 5, 6, 7, 8, 9, 10, or 11 R⁵ are moieties of formula (I):

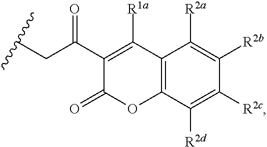

and the remaining R⁵ are C₅-C₁₀ alkyl or

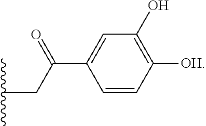 [SMILES: *CC(=O)c1ccc(O)c(O)c1]

In some embodiments, in formula (IXe), 1, 2, 3, 4, 5, 6, 7, 8, 9, 10, or 11 R⁵ are moieties of formula (I):

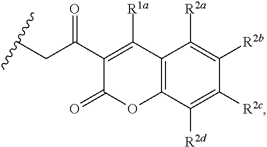

and the remaining R⁵ are C₁₀ alkyl or

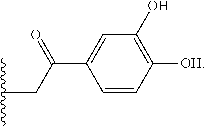 [SMILES: *CC(=O)c1ccc(O)c(O)c1]

In some embodiments, in formula (IXe), 1, 2, 3, 4, 5, 6, 7, 8, 9, 10, or 11 R⁵ are moieties of formula (I):

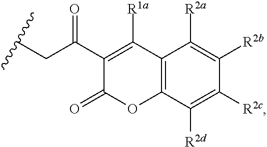

in formula (IXe), and the remaining R⁵ are C₆ alkyl or

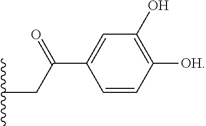 [SMILES: *CC(=O)c1ccc(O)c(O)c1]

In some embodiments, in formula (IXe), 1, 2, 3, 4, 5, 6, 7, 8, 9, 10, or 11 R⁵ are moieties of formula (I):

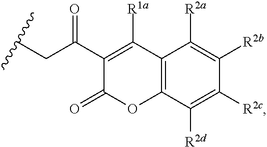

and the remaining R⁵ are C₁₂ alkyl or

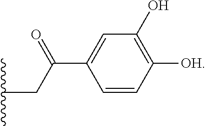 [SMILES: *CC(=O)c1ccc(O)c(O)c1]

Non-limiting examples of substructures of formula (IXe) include:

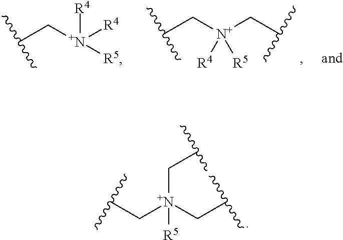

In some embodiments, in formula (IXe), R^{1a} is hydrogen. In some embodiments, in formula (IXe), R^{2a} is hydrogen. In some embodiments, in formula (IXe), R^{2b} is hydrogen. In some embodiments, in formula (IXe), R², is hydrogen. In some embodiments, in formula (I), R^{2d} is hydrogen. In some embodiments, in in formula (IXe), each R^{2a}, R^{2b}, R^{2c}, and R^{2d} is hydrogen.

In some embodiments, in formula (IXe), each R^{1a}, R^{2a}, R^{2b}, R^{2c}, and R^{2d} is hydrogen. In some embodiments, in formula (IXe), the moiety of formula (I) is a moiety of formula (10):

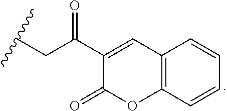 [SMILES: *CC(=O)c1cc2ccccc2oc1=O]

In some embodiments, in formula (IXe), in the molar ratio of the number of R⁵ moieties of formula (I) to the number of R⁵ optionally substituted alkyl groups to the number of R⁵

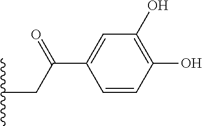 [SMILES: *CC(=O)c1ccc(O)c(O)c1]

groups is x:y:z, wherein about 0.05≤x≤0.4:0.2≤y≤0.9:0.05≤z≤0.4.

In some embodiments, in formula (IXe), the molar ratio of the number of R⁵ moieties of formula (I) to the number of R⁵ optionally substituted alkyl groups to the number of R⁵

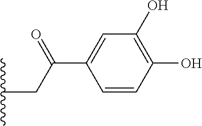 [SMILES: *CC(=O)c1ccc(O)c(O)c1]

groups is x:y:z, wherein about 0.08≤x≤0.12:0.76≤y≤0.84:0.08≤z≤0.12. In some embodiments, in formula (IXe), the PEI polymer is fully quaternized. In some embodiments, in formula (IXe), the molar ratio of the number of R⁵ moieties of formula (I) to the number of R⁵ optionally substituted alkyl groups to the number of R⁵

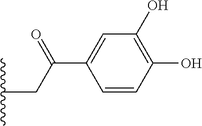 [SMILES: *CC(=O)c1ccc(O)c(O)c1]

groups is about 0.1:0.8:0.1. In some embodiments, in formula (IXe), the alkyl group is a C₁₀ group. In some embodiments, in formula (IXe), the alkyl group is a C₆ alkyl group.

In another aspect, the disclosure describes a PEI polymer comprising at least one moiety of formula (IXf), or substructures thereof:

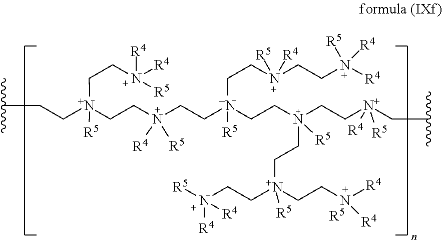

wherein in formula (IXf):
each R⁴ is independently optionally substituted alkyl; and
each R⁵ is independently optionally substituted alkyl, a moiety of formula (VII):

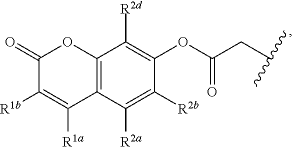

or a catechol moiety of

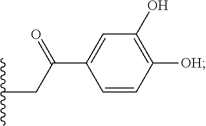 [SMILES: *CC(=O)c1ccc(O)c(O)c1]

each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl;
   with the proviso that at least one R⁵ is a moiety of formula (VIII):

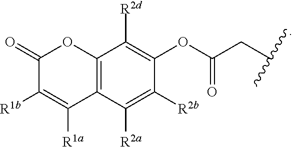

In some embodiments, in formula (IXf), each R⁴ is independently C₁-C₃ alkyl. In some embodiments, in formula (IXf), each R⁴ is methyl. In some embodiments, in formula (IXf), 1, 2, 3, 4, 5, 6, 7, 8, 9, 10, or 11 R⁵ are moieties of formula (VII):

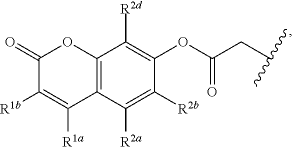

and the remaining R⁵ are C₅-C₁₀ alkyl or

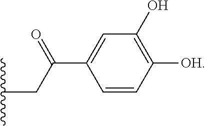 [SMILES: *CC(=O)c1ccc(O)c(O)c1]

In some embodiments, in formula (IXf), 1, 2, 3, 4, 5, 6, 7, 8, 9, 10, or 11 R⁵ are moieties of formula (VII):

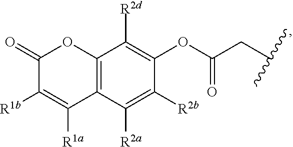

and the remaining R⁵ are C₁₀ alkyl or

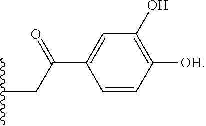 [SMILES: *CC(=O)c1ccc(O)c(O)c1]

In some embodiments, in formula (IXf), 1, 2, 3, 4, 5, 6, 7, 8, 9, 10, or 11 R⁵ are moieties of formula (VII):

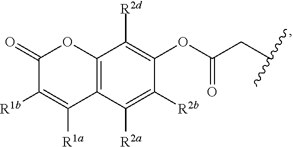

in formula (IXe), and the remaining R⁵ are C₆ alkyl or

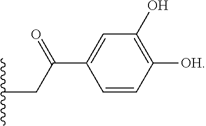 [SMILES: *CC(=O)c1ccc(O)c(O)c1]

In some embodiments, in formula (IXf), 1, 2, 3, 4, 5, 6, 7, 8, 9, 10, or 11 R⁵ are moieties of formula (VII):

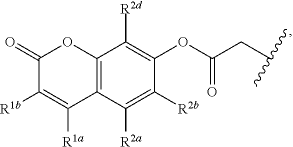

and the remaining R⁵ are C₁₂ alkyl or

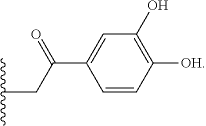 [SMILES: *CC(=O)c1ccc(O)c(O)c1]

Non-limiting examples of substructures of formula (IXf) include:

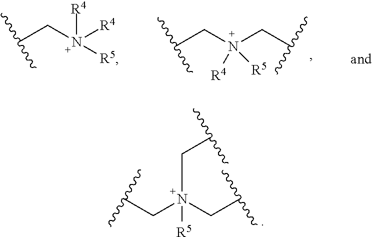

In some embodiments, in formula (IXf), R^{1a} is hydrogen. In some embodiments, in formula (VII), R^{1b} is hydrogen. In some embodiments, in formula (IXf), R^{2a} is hydrogen. In some embodiments, in formula (IXf), R^{2b} is hydrogen. In some embodiments, in formula (IXf), R^{2d} is hydrogen. In some embodiments, in formula (IXf), each R^{2a}, R^{2b}, R^{2c}, and R^{2d} is hydrogen. In some embodiments, in formula (IXf), each R^{1a}, R^{1b}, R^{2a}, R^{2b}, and R^{2d} is hydrogen.

In some embodiments, in formula (IXe), the moiety of formula (VII) is a moiety of formula (70):

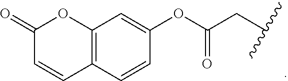 [SMILES: *CC(=O)Oc1ccc2ccc(=O)oc2c1]

In some embodiments, in formula (IXf), the molar ratio of the number of R⁵ moieties of formula (VII) to the number of R⁵ optionally substituted alkyl groups to the number of R⁵

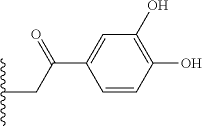 [SMILES: *CC(=O)c1ccc(O)c(O)c1]

groups is x:y:z, wherein about 0.05≤x≤0.4:0.2≤y≤0.9:0.05≤z≤0.4. In some embodiments, in formula (IXf), the molar ratio of the number of R⁵ moieties of formula (VII) to the number of R⁵ optionally substituted alkyl groups to the number of R⁵

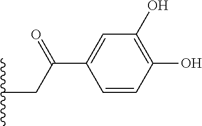 [SMILES: *CC(=O)c1ccc(O)c(O)c1]

groups is x:y:z, wherein about 0.08≤x≤0.12:0.76≤y≤0.84:0.08≤z≤0.12. In some embodiments, in formula (IXf), the PEI polymer is fully quaternized. In some embodiments, in formula (IXf), the molar ratio of the number of R⁵ moieties of formula (VII) to the number of R⁵ optionally substituted alkyl groups to the number of R⁵

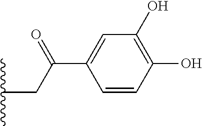 [SMILES: *CC(=O)c1ccc(O)c(O)c1]

groups is about 0.1:0.8:0.1. In some embodiments, in formula (IXf), the alkyl group is a C₁₀ group. In some embodiments, in formula (IXf), the alkyl group is a C₆ alkyl group.

In another aspect, the disclosure describes a PEI polymer comprising at least one moiety of formula (IXg), or substructures thereof:

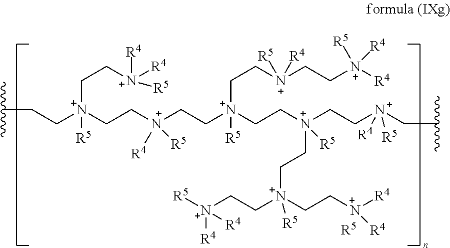

wherein in formula (IXg):
   each R⁴ is independently optionally substituted alkyl; and
   each R⁵ is independently optionally substituted alkyl, a moiety of formula (I):

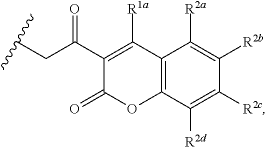

or a silane moiety of formula (9):

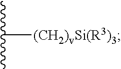

R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R³ is independently optionally substituted alkoxy; and
v is an integer from 3 to 10;
   with the proviso that at least one R⁵ is a moiety of formula (I):

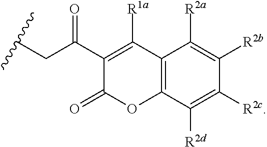

In some embodiments, in formula (IXg), each R⁴ is independently C₁-C₃ alkyl. In some embodiments, in formula (IXg), each R⁴ is methyl. In some embodiments, in formula (IXe), 1, 2, 3, 4, 5, 6, 7, 8, 9, 10, or 11 R⁵ are moieties of formula (I):

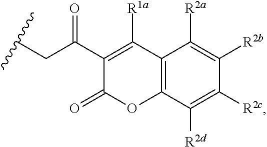

and the remaining R⁵ are C₅-C₁₀ alkyl or a silane moiety of formula (9). In some embodiments, in formula (IXg), 1, 2, 3, 4, 5, 6, 7, 8, 9, 10, or 11 R⁵ are moieties of formula (I):

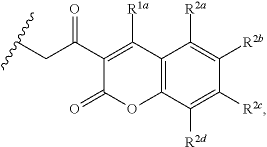

and the remaining R⁵ are C₁₀ alkyl or a silane moiety of formula (9). In some embodiments, in formula (IXg), 1, 2, 3, 4, 5, 6, 7, 8, 9, 10, or 11 R⁵ are moieties of formula (I):

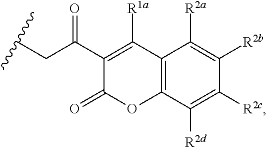

in formula (IXg), and the remaining R⁵ are C₆ alkyl or a silane moiety of formula (9). In some embodiments, in formula (IXg), 1, 2, 3, 4, 5, 6, 7, 8, 9, 10, or 11 R⁵ are moieties of formula (I):

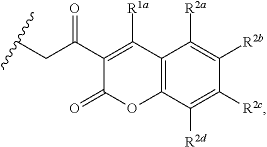

and the remaining R⁵ are C₁₂ alkyl or a silane moiety of formula (9).

Non-limiting examples of substructures of formula (IXg) include:

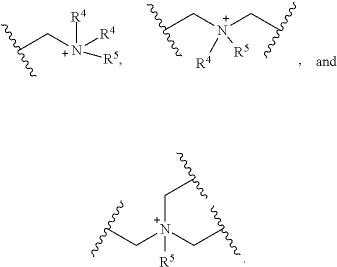

In some embodiments, in formula (IXg), R^{1a} is hydrogen. In some embodiments, in formula (IXg), R² is hydrogen. In some embodiments, in formula (IXg), R^{2b} is hydrogen. In some embodiments, in formula (IXg), R^{2e} is hydrogen. In some embodiments, in formula (IXg), R^{2d} is hydrogen. In some embodiments, in in formula (IXg), each R^{2a}, R^{2b}, R^{2c}, and R^{2d} is hydrogen. In some embodiments, in formula (IXg), each R^{1a}, R^{2a}, R^{2b}, R^{2c}, and R^{2d} is hydrogen.

In some embodiments, in formula (IXg), the moiety of formula (I) is a moiety of formula (10):

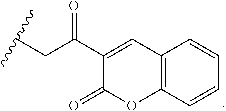 [SMILES: *CC(=O)c1cc2ccccc2oc1=O]

In some embodiments, in formula (9), v is an integer from 3 to 5. In some embodiments, in formula (9), v is 3. In some embodiments, in formula (9), each R³ is C₁-C₃ alkoxy. In some embodiments, each R³ is methoxy. In some embodiments, in formula (IV), v is 3 and each R³ is methoxy. In some embodiments, in formula (9), v is an integer from 3 to 5, optionally v is 3, and each R³ is C₁-C₃ alkoxy, optionally R³ is methoxy. In some embodiments, in formula (IXg), the moiety of formula (9) is the following moiety:

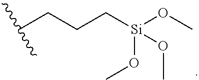 [SMILES: *CCC[Si](OC)(OC)OC]

In some embodiments, in formula (IXg), the molar ratio of the number of R⁵ moieties of formula (I) to the number of R⁵ optionally substituted alkyl groups to the number of R⁵ groups of formula (9) is x:y:z, wherein about 0.05≤x≤0.4:0.2≤y≤0.9:0.05≤z≤0.4. In some embodiments, in formula (IXg), the molar ratio of the number of R⁵ moieties of formula (I) to the number of R⁵ optionally substituted alkyl groups to the number of R⁵ groups of formula (9) is x:y:z, wherein about 0.08≤x≤0.12:0.76≤y≤0.84:0.08≤z≤0.12. In some embodiments, in formula (IXg), the PEI polymer is fully quaternized. In some embodiments, in formula (IXg), the molar ratio of the number of R⁵ moieties of formula (I) to the number of R⁵ optionally substituted alkyl groups to the number of number of R⁵ groups of formula (9) is about 0.1:0.8:0.1. In some embodiments, in formula (IXg), the alkyl group is a C₁₀ group. In some embodiments, in formula (IXg), the alkyl group is a C₆ alkyl group.

In another aspect, the disclosure describes a PEI polymer comprising at least one moiety of formula (IXh), or substructures thereof:

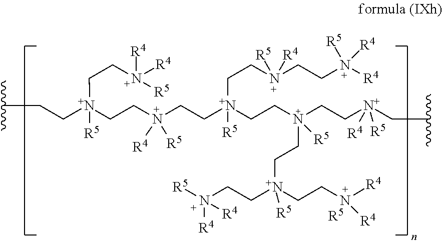

wherein in formula (IXh):
   each R⁴ is independently optionally substituted alkyl; and
   each R⁵ is independently optionally substituted alkyl, a moiety of formula (VII):

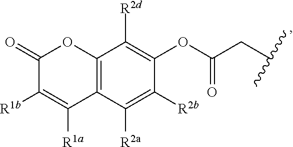

or a silane moiety of formula (9):

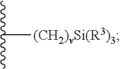

each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R³ is independently optionally substituted alkoxy; and
v is an integer from 3 to 10;
with the proviso that at least one R⁵ is a moiety of formula (VII):

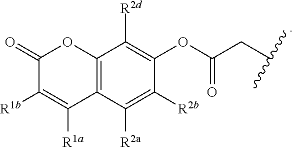

In some embodiments, in formula (IXh), each R⁴ is independently C₁-C₃ alkyl. In some embodiments, in formula (IX), each R⁴ is methyl. In some embodiments, in formula (IX), 1, 2, 3, 4, 5, 6, 7, 8, 9, 10, or 11 R⁵ are moieties of formula (VII):

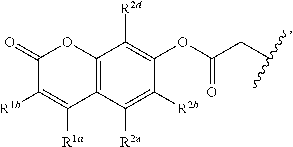

and the remaining R⁵ are C₅-C₁₀ alkyl or a silane moiety of formula (9). In some embodiments, in formula (IXh), 1, 2, 3, 4, 5, 6, 7, 8, 9, 10, or 11 R⁵ are moieties of formula (VII):

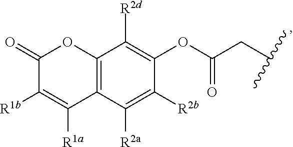

and the remaining R⁵ are C₁₀ alkyl or a silane moiety of formula (9).

In some embodiments, in formula (IXh), 1, 2, 3, 4, 5, 6, 7, 8, 9, 10, or 11 R⁵ are moieties of formula (VII):

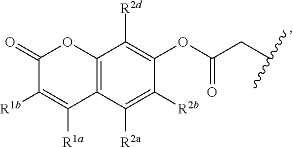

in formula (IXh), and the remaining R⁵ are C₆ alkyl or a silane moiety of formula (9). In some embodiments, in formula (IXh), 1, 2, 3, 4, 5, 6, 7, 8, 9, 10, or 11 R⁵ are moieties of formula (VII):

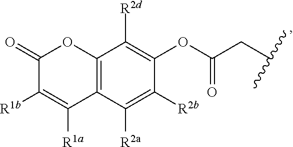

and the remaining R⁵ are C₁₂ alkyl or a silane moiety of formula (9).

Non-limiting examples of substructures of formula (IXh) include:

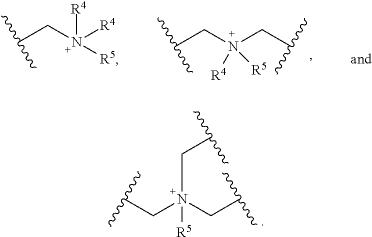

In some embodiments, in formula (IXh), R^{1a} is hydrogen. In some embodiments, in formula (IXh), R^{1b} is hydrogen. In some embodiments, in formula (IXh), R^{2a} is hydrogen. In some embodiments, in formula (IXh), R^{2b} is hydrogen. In some embodiments, in formula (IXh), R^{2d} is hydrogen. In some embodiments, in formula (IXf), each R^{2a}, R^{2b}, R^{2c}, and R^{2d} is hydrogen.

In some embodiments, in formula (IXh), each R^{1a}, R^{1b}, R^{2a}, R^{2b}, and R^{2d} is hydrogen.

In some embodiments, in formula (IXh), the moiety of formula (VII) is a moiety of formula (70):

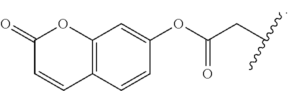 [SMILES: *CC(=O)Oc1ccc2ccc(=O)oc2c1]

In some embodiments, in formula (9), v is an integer from 3 to 5. In some embodiments, in formula (9), v is 3. In some embodiments, in formula (9), each R³ is C₁-C₃ alkoxy. In some embodiments, each R³ is methoxy. In some embodiments, in formula (IV), v is 3 and each R³ is methoxy. In some embodiments, in formula (9), v is an integer from 3 to 5, optionally v is 3, and each R³ is C₁-C₃ alkoxy, optionally R³ is methoxy. In some embodiments, in formula (IXg), the moiety of formula (9) is the following moiety:

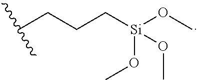 [SMILES: *CCC[Si](OC)(OC)OC]

In some embodiments, in formula (IXh), the molar ratio of the number of R⁵ moieties of formula (VII) to the number of R⁵ optionally substituted alkyl groups to the number of R⁵ groups of formula (9) is :y:z, wherein about 0.05≤x≤0.4:0.2≤y≤0.9:0.05≤z≤0.4. In some embodiments, in formula (IXh), the molar ratio of the number of R⁵ moieties of formula (VII) to the number of R⁵ optionally substituted alkyl groups to the number of R⁵ groups of formula (9) is x:y:z, wherein about 0.08≤x≤0.12:0.76≤y 0.84:0.08≤z≤0.12. In some embodiments, in formula (IXh), the PEI polymer is fully quaternized. In some embodiments, in formula (IXh), the molar ratio of the number of R⁵ moieties of formula (VII) to the number of R⁵ optionally substituted alkyl groups to the number of R⁵ groups of formula (9) is about 0.1:0.8:0.1. In some embodiments, in formula (IXh), the alkyl group is a C₁₀ group. In some embodiments, in formula (IXh), the alkyl group is a C₆ alkyl group.

The amount of a particular moiety or monomer, whether the relative amount or a quantitative amount, present in a polymer or copolymer as described herein, can be determined and described using methods as understood by one of ordinary skill in the art. In one embodiment, the amount of each moiety present in a polymer is described by its molar ratio. In some embodiments, the molar ratio is 0.05, 0.06, 0.07, 0.08, 0.09, 0.11, 0.11, 0.12, 0.13, 0.14, 0.15, 0.16, 0.17. 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50. In some embodiments, a polymer comprises two different moieties, and the moieties are present in a molar ratio of 0.05≤x≤0.5:(1-x) or 0.05≤x≤0.2:(1−x). In some embodiments, x is 0.05, 0.06, 0.07, 0.08, 0.09, 0.11, 0.11, 0.12, 0.13, 0.14, 0.15, 0.16, 0.17. 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50. In some embodiments, a polymer comprises three different moieties, and the moieties are present in a molar ratio of x:y:z, wherein x+y+z=1.

In some embodiments, the polymer at least one moiety of formula (Ia), at least one moiety of formula (3), and at least one moiety of formula (4), and the moieties of formula (Ia), moieties of formula (3), and moieties of formula (4) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (Iaa), at least one moiety of formula (3), and at least one moiety of formula (4), and the moieties of formula (Iaa), moieties of formula (3), and moieties of formula (4) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (VIIa), at least one moiety of formula (3), and at least one moiety of formula (4), and the moieties of formula (VIIa), moieties of formula (3), and moieties of formula (4) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (VIIb), at least one moiety of formula (3), and at least one moiety of formula (4), and the moieties of formula (VIIb), moieties of formula (3), and moieties of formula (4) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (Ia), at least one moiety of formula (5), and at least one moiety of formula (6), and the moieties of formula (Ia), moieties of formula (5), and moieties of formula (6) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (Iaa), at least one moiety of formula (5), and at least one moiety of formula (6), and the moieties of formula (Iaa), moieties of formula (5), and moieties of formula (6) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (VIIa), at least one moiety of formula (5), and at least one moiety of formula (6), and the moieties of formula (VIIa), moieties of formula (5), and moieties of formula (6) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (VIIb), at least one moiety of formula (5), and at least one moiety of formula (6), and the moieties of formula (VIIb), moieties of formula (5), and moieties of formula (6) are present in a molar ratio of x:y:z, wherein 0.1≤x≤0.2:0.1≤y≤0.2:0.6≤z≤0.8, and x+y+z=1.

In some embodiments, the polymer comprises at least one moiety of formula (Ib) at least one moiety of formula (7), and at least one moiety of formula (8), and the moieties of formula (Ib), moieties of formula (7), and moieties of formula (8) are present in a molar ratio of x:y:z, wherein 0.1≤x≤0.2:0.6≤y≤0.8:0.1≤z≤0.2, and x+y+z=1.

In some embodiments, the polymer comprises at least one moiety of formula (Ibb) at least one moiety of formula (7), and at least one moiety of formula (8), and the moieties of formula (Ibb), moieties of formula (7), and moieties of formula (8) are present in a molar ratio of x:y:z, wherein 0.1≤x≤0.2:0.6≤y≤0.8:0.1≤z≤0.2, and x+y+z=1.

In some embodiments, the polymer comprises at least one moiety of formula (IIaa) at least one moiety of formula (7), and at least one moiety of formula (8), and the moieties of formula (IIaa), moieties of formula (7), and moieties of formula (8) are present in a molar ratio of x:y:z, wherein 0.1≤x≤0.2:0.6≤y≤0.8:0.1≤z≤0.2, and x+y+z=1.

In some embodiments, the polymer comprises at least one moiety of formula (IIa) at least one moiety of formula (7), and at least one moiety of formula (8), and the moieties of formula (IIa), moieties of formula (7), and moieties of formula (8) are present in a molar ratio of x:y:z, wherein 0.1≤x≤0.2:0.6≤y≤0.8:0.1≤z≤0.2, and x+y+z=1.

In some embodiments, the polymer comprises at least one moiety of formula (IIaa) at least one moiety of formula (7), and at least one moiety of formula (13), and the moieties of formula (IIaa), moieties of formula (7), and moieties of formula (13) are present in a molar ratio of x:y:z, wherein 0.1≤x≤0.2:0.6≤y≤0.8:0.1≤z≤0.2, and x+y+z=1.

In some embodiments, the polymer comprises at least one moiety of formula (IIa) at least one moiety of formula (7), and at least one moiety of formula (13), and the moieties of formula (IIa), moieties of formula (7), and moieties of formula (13) are present in a molar ratio of x:y:z, wherein 0.1≤x≤0.2:0.6≤y≤0.8:0.1≤z≤0.2, and x+y+z=1.

Any polymer is contemplated for use within the disclosure, as would be understood by one of ordinary skill in the art. In some embodiments, the polymer is a random copolymer. In some embodiments, the polymer comprises polyvinylpyridine (PVP), polyvinylbenzylchloride, polyethyleneimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide) and poly(diallyldimethylammonium). In some embodiments, the polymer further comprises polyvinylpyridine (PVP) or polyethyleneimine (PEI). In some embodiments, the polymer further comprises an optionally substituted C₄-C₂₂ alkyl group. In some embodiments, the polymer further comprises an optionally substituted C₃-C₂₂ alkyne In some embodiments, the polymer further comprises an optionally substituted C₃-C₂₂ terminal alkyne. In some embodiments, the polymer is fully quaternized. In some embodiments, the polymer is partially quaternized. In some embodiments, the ratio of quaternized to unquaternized moieties (N⁺/N ratio) is about 30% to about 50%.

In one aspect, the disclosure provides a compound comprising a detectable moiety of the disclosure.

In one aspect, the disclosure provides a compound of formula (III):

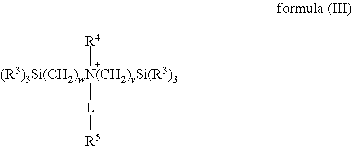

wherein in formula (III):
L is a single bond or a linking group;
each R³ is independently optionally substituted alkoxy;
R⁴ is independently optionally substituted alkyl;
R⁵ is a detectable moiety;
v is an integer from 3 to 10; and
w is an integer from 3 to 10.

In one aspect, the disclosure provides a compound of formula (IIIa):

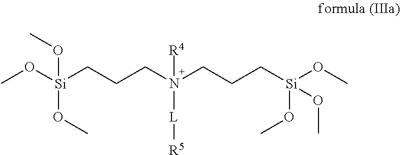

wherein in formula (IIIa):
L is a single bond or a linking group;
R⁴ is optionally substituted alkyl; and
R⁵ is a detectable moiety.

In one aspect, the disclosure provides a compound of formula (IIIb):

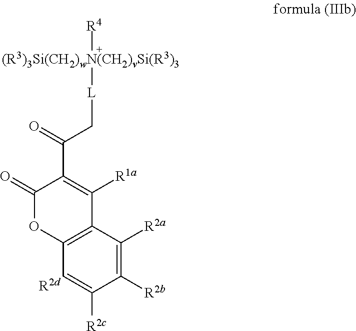

wherein in formula (IIIb):
L is a single bond or a linking group;
R^{1a} is independently optionally selected from hydrogen and alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R³ is independently optionally substituted alkoxy; and
R⁴ is optionally substituted alkyl.

In some embodiments, in formula (IIIb), R^{1a} is hydrogen. In some embodiments, in formula (IIaa), R^{1a} is C₁-C₃ alkyl. In some embodiments, in formula (IIIb), R^{1a} is methyl. In some embodiments, in formula (IIIb), R^{2a} is hydrogen. In some embodiments, in formula (IIaa), R^{2b} is hydrogen. In some embodiments, in formula (IIIb), R^{2c} is hydrogen. In some embodiments, in formula (IIIb), R^{2d} is hydrogen. In some embodiments, in formula (IIIb), each R^{1a}, R^{2a}, R^{2b}, R^{2c}, and R^{2d} is hydrogen. In some embodiments, in formula (IIIb), R^{1a} is methyl and each R^{2a}, R^{2b}, R^{2c}, and R^{2d} is hydrogen. In some embodiments, in formula (III), (IIIa), or (IIIb), R⁴ is C₁-C₁₀ alkyl, C₁-C₅ alkyl, C₁-C₃ alkyl, or C₅-C₁₀ alkyl. In some embodiments, in formula (III), (IIIa), or (IIIb), R⁴ is methyl. In some embodiments, in formula (III), (IIIa), or (IIIb), R⁴ is ethyl. In some embodiments, in formula (III), (IIIa), or (IIIb), R⁴ is propyl. In some embodiments, in formula (III), (IIIa), or (IIIb), R⁴ is —C₁₀H₂₁. In some embodiments, in formula (III), (IIIa), or (IIIb), R⁴ is C₁-C₃ alkyl. In some embodiments, in formula (III), (IIIa), or (IIIb), R⁴ is C₅-C₁₀ alkyl.

In some embodiments, in formula (IIIb), each R^{1a}, R^{2a}, R^{2b}, R^{2c}, and R^{2d} is hydrogen, and each R⁴ is C₁-C₃ alkyl, optionally each R⁴ is ethyl. In some embodiments, in formula (IIIb), R^{1a} is C₁-C₃ alkyl, optionally R^{1a} is methyl, each R^{2a}, R^{2b}, R^{2c}, and R^{2d} is hydrogen, and each R⁴ is C₁-C₃ alkyl, optionally each R⁴ is ethyl.

In some embodiments, in formula (III), (IIIa), or (IIIb), L is a single bond. In some embodiments, formula (III), (IIIa), or (IIIb), L is a linking group. The linking group may be any organic moiety, as would be understood by one of ordinary skill in the art. In some embodiments, the linking group comprises an optionally substituted alkyl group and a carbonyl group. In some embodiments, formula (III), (IIIa), or (IIIb), L is selected from

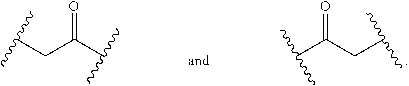 [SMILES: *CC(*)=O ; *CC(*)=O]

In some embodiments, formula (III), (IIIa), or (IIIb), the linking group comprises optionally substituted amino, optionally substituted heterocyclyl, carboxyl, or optionally substituted thiol. In some embodiments, the linking group comprises a primary amino group, a secondary amino group, or a tertiary amino group. In some embodiments, formula (III), (IIIa), or (IIIb), the organic linker comprises dimethylamino, diethylamino, —C(O)O—, —S—, diethylcarboxylate, acetyl, optionally substituted triazole group, or optionally substituted tetrazole group.

In some embodiments, in formula (III), (IIIa), or (IIIb), v is an integer from 3 to 5. In some embodiments, in formula (III), (IIIa), or (IIIb), v is 3. In some embodiments, in formula (III), (IIIa), or (IIIb), w is an integer from 3 to 5. In some embodiments, in formula (III), (IIIa), or (IIIb), w is 3. In some embodiments, in formula (III), (IIIa), or (IIIb), each R³ is C₁-C₃ alkoxy. In some embodiments, in formula (III), (IIIa), or (IIIb), each R³ is methoxy. In some embodiments, in formula (III), (IIIa), or (IIIb), v is an integer from 3 to 5, optionally v is 3, w is an integer from 3 to 5, optionally w is 3, and each R³ is C₁-C₃ alkoxy, optionally R³ is methoxy.

In some embodiments, in formula (III), (IIIa), or (IIIb), L is a single bond. In some embodiments, in formula (III), (IIIa), or (IIIb), L is selected from and

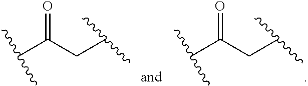 [SMILES: *CC(*)=O ; *CC(*)=O]

in some embodiments, in formula (III), (IIIa), or (IIIb), R⁵ comprises a coumarin moiety. In some embodiments, in formula (III) or (IIIa), R⁵ is a detectable moiety having formula (I), formula (11), formula (II), formula (10), formula (12), formula (20), formula (21).

In some embodiments, the compound of formula (III) is a compound having formula (300):

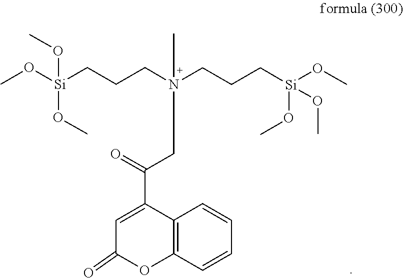

In some embodiments, the compound of formula (III) is a compound having formula (301):

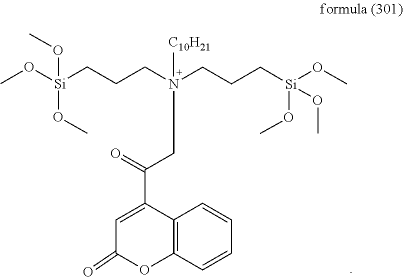

In one aspect, the disclosure provides a compound of formula (IV):

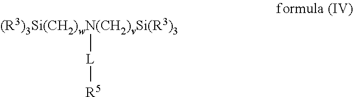

wherein in formula (IV):
L is a single bond or a linking group;
each R³ is independently optionally substituted alkoxy;
R⁵ is a detectable moiety;
v is an integer from 3 to 10; and
w is an integer from 3 to 10.

In one aspect, the disclosure provides a compound of formula (IVa):

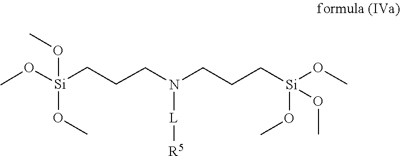

wherein in formula (IVa):
L is a single bond or a linking group; and
R⁵ is a detectable moiety.

In some embodiments, the disclosure provides a compound of formula (IVb):

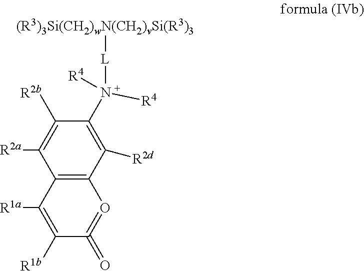

wherein in formula (IVb):
L is a single bond or a linking group;
each R³ is independently optionally substituted alkoxy;
each R⁴ is independently optionally substituted alkyl;
v is an integer from 3 to 10; and
w is an integer from 3 to 10.

In some embodiments, in formula (IV), (IVa), or (IVb), L is a single bond. In some embodiments, in formula (IV), (IVa), or (IVb), L is a linking group. The linking group may be any organic moiety, as would be understood by one of ordinary skill in the art. In some embodiments, the linking group comprises an optionally substituted alkyl group and a carbonyl group. In some embodiments, in formula (IV), (IVa), or (IVb), L is selected from

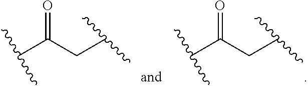 [SMILES: *CC(*)=O ; *CC(*)=O]

In some embodiments, the linking group comprises optionally substituted amino, optionally substituted heterocyclyl, carboxyl, or optionally substituted thiol. In some embodiments, the linking group comprises a primary amino group, a secondary amino group, or a tertiary amino group. In some embodiments, in formula (IV), (IVa), or (IVb), the organic linker comprises dimethylamino, diethylamino, —C(O)O—, —S—, diethylcarboxylate, acetyl, optionally substituted triazole group, or optionally substituted tetrazole group.

In some embodiments, in formula (IV) or (IVb), v is an integer from 3 to 5. In some embodiments, in formula (IV) or (IVb), v is 3. In some embodiments, in formula (IV) or (IVb), w is an integer from 3 to 5. In some embodiments, in formula (IV) or (IVb), w is 3. In some embodiments, in formula (IV) or (IVb), each R³ is C₁-C₃ alkoxy. In some embodiments, in formula (IV) or (IVb), each R³ is methoxy. In some embodiments, in formula (IV) or (IVb), v is an integer from 3 to 5, optionally v is 3, w is an integer from 3 to 5, optionally w is 3, and each R³ is C₁-C₃ alkoxy, optionally R³ is methoxy.

In some embodiments, in formula (IVb), R⁴ is C₁-C₁₀ alkyl, C₁-C₅ alkyl, C₁-C₃ alkyl, or C₅-C₁₀ alkyl. In some embodiments, in formula (IVb), R⁴ is methyl. In some embodiments, in formula (IVb), R⁴ is ethyl. In some embodiments, in formula (IVb), R⁴ is propyl. In some embodiments, in formula (IVb), R⁴ is —C₁₀H₂₁. In some embodiments, in formula (IVb), R⁴ is C₁-C₃ alkyl. In some embodiments, in formula (IVb), R⁴ is C₅-C₁₀ alkyl.

In some embodiments, in formula (IV), (IVa), or (IVb), L is a single bond. In some embodiments, in formula (IV), (IVa), or (IVb), L is selected from

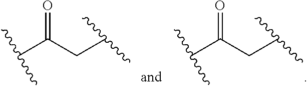 [SMILES: *CC(*)=O ; *CC(*)=O]

In some embodiments, in formula (IV), (IVa), or (IVb), R⁵ comprises a coumarin moiety. In some embodiments, in formula (IV), (IVa), or (IVb), R⁵ is a detectable moiety having formula (I), formula (11), formula (II), formula (10), formula (12), formula (20), formula (21).

In some embodiments, the compound of formula (IV) is a compound having formula (400):

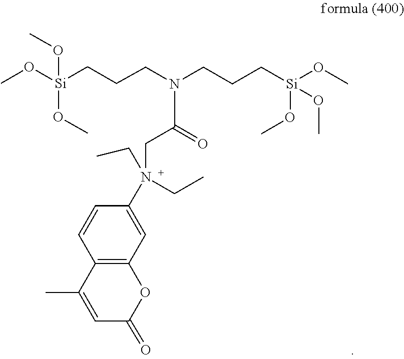

In one aspect, the disclosure provides a compound of formula (VI):

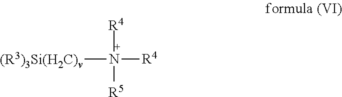

wherein in formula (VI):
L is a single bond or a linking group;
each R³ is independently optionally substituted alkoxy;
each R⁴ is independently optionally substituted alkyl;
R⁵ is a detectable moiety; and
v is an integer from 3 to 10.

In one aspect, the disclosure provides a compound of formula (VIa):

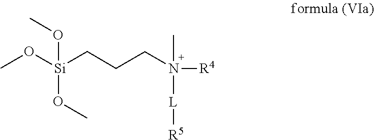

wherein in formula (VIa):
L is a single bond or a linker;
each R⁴ is independently optionally substituted alkyl; and
R⁵ is a detectable moiety.

In one aspect, the disclosure provides a compound of formula (VIb):

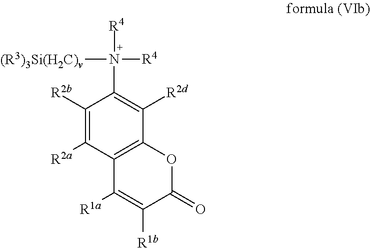

wherein in formula (VIb):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkoxy;
each R⁴ is independently optionally substituted alkyl; and
v is an integer from 3 to 10.

In one aspect, the disclosure provides a compound of formula (Ic):

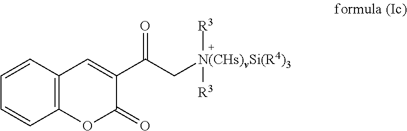

wherein in formula (Ic):
each R³ is independently optionally substituted alkyl;
each R⁴ is independently optionally substituted alkoxy; and
v is an integer from 3 to 10.

In some embodiments, in formula (Ic), each R³ is independently C₁-C₂₀ alkyl, C₁-C18 alkyl, C₁₆-C18 alkyl, C₁-C₅ alkyl, C₁-C₃ alkyl, or C₅-C₁₀ alkyl. In some embodiments, in formula (Ic), each R³ is methyl. In some embodiments, in formula (Ic), one R³ is C₁-C₃ alkyl and the other R³ is C₁₆-C18 alkyl. In some embodiments, in formula (Ic), one R³ is methyl and the other R³ is —C₁₈H₃₇. In some embodiments, in formula (Ic), v is an integer from 3 to 5. In some embodiments, in formula (Ic), v is 3. In some embodiments, in formula (Ic), each R⁴ is C₁-C₅ alkoxy or C₁-C₃ alkoxy. In some embodiments, in formula (Ic), each R⁴ is methoxy. In some embodiments, in formula (Ic), each R³ is methyl, v is 3, and each R⁴ is methoxy. In some embodiments, in formula (Ic), each R³ is independently C₁-C₂₀ alkyl or C₁-C18 alkyl, optionally one R³ is C₁-C₃ alkyl and the other R³ is C₁₆-C18 alkyl, optionally one R³ is methyl and the other R³ is —C₁₈H₃₇, v is 3, and each R⁴ is C₁-C₃ alkoxy, optionally each R⁴ is methoxy.

In some embodiments, in formula (VIb), R^{1a} is hydrogen. In some embodiments, in formula (VIb), R^{1a} is C₁-C₃ alkyl. In some embodiments, in formula (VIb), R^{1a} is methyl. In some embodiments, in formula (VIb), R^{1b} is hydrogen. In some embodiments, in formula (VIb), R^{2a} is hydrogen. In some embodiments, in formula (VIb), R^{2b} is hydrogen. In some embodiments, in formula (VIb), R^{2d} is hydrogen. In some embodiments, in formula (VIb), each R^{1a}, R^{1b} R^{2a}, R^{2b}, and R^{2d} is hydrogen. In some embodiments, in formula (VIb), R^{1a} is methyl and each R^{1b} R^{2a}, R^{2b}, and R^{2d} is hydrogen. In some embodiments, in formula (VIb), each R³ is C₁-C₃ alkyl. In some embodiments, in formula (VIb), each R³ is ethyl. In some embodiments, in formula (VIb), R^{1a} is C₁-C₃ alkyl, optionally R^{1a} is methyl, each R^{1b} R^{2a}, R^{2b}, and R^{2d} is hydrogen, and each R³ is C₁-C₃ alkyl, optionally each R³ is ethyl.

In some embodiments, in formula (VI) or (VIa), L is a single bond. In some embodiments, formula (VI) or (VIa), L is a linking group. The linking group may be any organic moiety, as would be understood by one of ordinary skill in the art. In some embodiments, the linking group comprises an optionally substituted alkyl group and a carbonyl group. In some embodiments, formula (VI) or (VIa), L is selected from

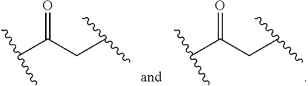 [SMILES: *CC(*)=O ; *CC(*)=O]

In some embodiments, formula (VI) or (VIa), the linking group comprises optionally substituted amino, optionally substituted heterocyclyl, carboxyl, or optionally substituted thiol. In some embodiments, the linking group comprises a primary amino group, a secondary amino group, or a tertiary amino group. In some embodiments, formula (VI) or (VIa), the organic linker comprises dimethylamino, diethylamino, —C(O)O—, —S—, diethylcarboxylate, acetyl, optionally substituted triazole group, or optionally substituted tetrazole group.

In some embodiments, in formula (VI), (VIa), or (VIb), R⁴ is C₁-C₁₀ alkyl, C₁-C₅ alkyl, C₁-C₃ alkyl, or C₅-C₁₀ alkyl. In some embodiments, in formula (VI), (VIa), or (VIb), R⁴ is methyl. In some embodiments, in formula (VI), (VIa), or (VIb), R⁴ is ethyl. In some embodiments, in formula (VI), (VIa), or (VIb), R⁴ is propyl. In some embodiments, in formula (VI), (VIa), or (VIb), R⁴ is —C₁₀H₂₁. In some embodiments, in formula (VI), (VIa), or (VIb), R⁴ is C₁-C₃ alkyl. In some embodiments, in formula (VI), (VIa), or (VIb), R⁴ is C₅-C₁₀ alkyl.

In some embodiments, in formula (VI) or (VIb), v is an integer from 3 to 5. In some embodiments, in formula (VI) or (VIb), v is 3. In some embodiments, in formula formula (VI) or (VIb), w is an integer from 3 to 5. In some embodiments, in formula (VI) or (VIb), w is 3. In some embodiments, in formula (VI) or (VIb), each R³ is C₁-C₃ alkoxy. In some embodiments, in formula (VI) or (VIb), each R³ is methoxy. In some embodiments, in formula (VI) or (VIb), v is an integer from 3 to 5, optionally v is 3, w is an integer from 3 to 5, optionally w is 3, and each R³ is C₁-C₃ alkoxy, optionally R³ is methoxy.

In some embodiments, in formula (VI) or (VIa), L is a single bond. In some embodiments, in formula (VI) or (VIa), L is selected from

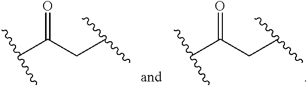 [SMILES: *CC(*)=O ; *CC(*)=O]

In some embodiments, in formula (VI) or (VIa), R⁵ comprises a coumarin moiety. In some embodiments, in formula (VI) or (VIa), R⁵ is a detectable moiety having formula (I), formula (11), formula (II), formula (10), formula (12), formula (20), formula (21).

In some embodiments, the compound of formula (VI) is a compound having formula (600):

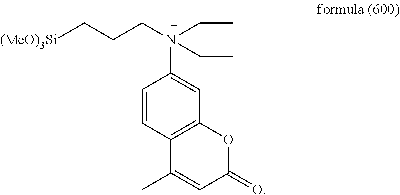

In some embodiments, the compound of formula (Ic) is a compound having formula (6001):

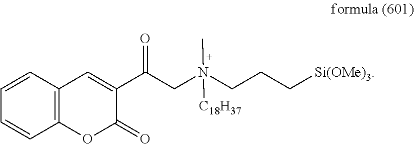

In one aspect, the disclosure provides a compound of formula (VIII):

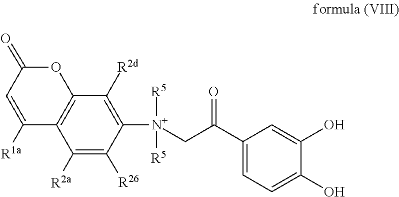

wherein in formula (VIII):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R⁵ is independently optionally substituted alkyl.

In some embodiments, in formula (VIII), R^{1a} is hydrogen. In some embodiments, in formula (VIII), R^{1a} is C₁-C₃ alkyl. In some embodiments, in formula (VIII), R^{1a} is methyl. In some embodiments, in formula (VIII), R^{1b} is hydrogen. In some embodiments, in formula (VIII), R^{2a} is hydrogen. In some embodiments, in formula (VIII), R^{2b} is hydrogen. In some embodiments, in formula (VIII), R^{2d} is hydrogen. In some embodiments, in formula (VIII), each R^{1a}, R^{1b} R^{2a}, R^{2b}, and R^{2d} is hydrogen. In some embodiments, in formula (VIII), R^{1a} is methyl and each R^{1b} R^{2a}, R^{2b}, and R^{2d} is hydrogen. In some embodiments, in formula (VIII), each R⁵ is C₁-C₅ alkyl, or C₁-C₃ alkyl. In some embodiments, in formula (VIII), each R⁵ is ethyl. In some embodiments, in formula (VIII), R^{1a} is C₁-C₃ alkyl, optionally R^{1a} is methyl, each R^{1b} R^{2a}, R^{2b} and R^{2d} is hydrogen, and each R³ is C₁-C₃ alkyl, optionally each R⁵ is ethyl.

In one aspect, the disclosure provides a compound of formula (VIIIa):

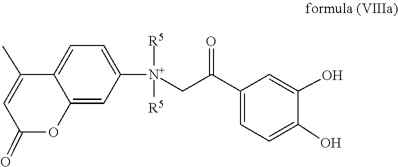

wherein in formula (VIIIa):
each R⁵ is independently optionally substituted alkyl.

In some embodiments, in formula (VIIIa), each R⁵ is C₁-C₅ alkyl, or C₁-C₃ alkyl. In some embodiments, in formula (VIIIa), each R⁵ is ethyl.

In one aspect, the disclosure provides a compound of formula (800):

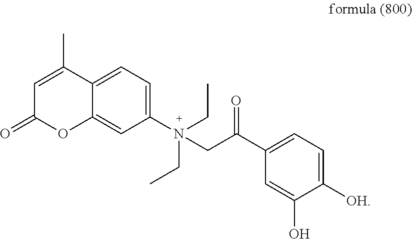

In one aspect, the present invention provides a method for preparing a polymer comprising a detectable moiety. In some embodiments, the method comprises treating a precursor polymer with a reactive detectable compound. Any compound that comprises one or more detectable moieties and is capable of forming a covalent bond with a polymer is contemplated by the invention as a reactive detectable compound. In some embodiments, the detectable compound is covalently attached to the precursor polymer after treatment.

In some embodiments, the detectable moiety comprises one or more moieties selected from a fluorescent moiety, a phosphorescent moiety, and a luminescent moiety. In some embodiments, the fluorescent moiety is selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety. In some embodiments, detectable compound comprises a coumarin moiety.

In one aspect, the disclosure provides a reactive detectable compound of formula (100):

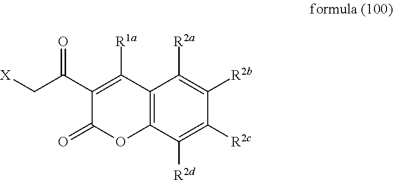

wherein in formula (100):
X is halogen or N(R³)₂;
R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
R³ is at independently at each occurrence alkyl.

In some embodiments, X is bromine.

In some embodiments, R^{1a} is hydrogen.

In some embodiments, R^{2a} is hydrogen. In some embodiments, R^{2b} is hydrogen. In some embodiments, R^{2c} is hydrogen. In some embodiments, R^{2d} is hydrogen. In some embodiments, each R^{2a}, R^{2b}, R^{2c}, and R^{2d} is hydrogen.

In one aspect, the disclosure provides a reactive detectable compound of formula (200):

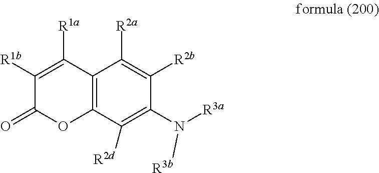

wherein in formula (200):
each R^{1a} and R^{1a} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

In some embodiments, R^{1a} is hydrogen. In some embodiments, R^{1a} is C₁-C₃ alkyl. In some embodiments, R^{1a} is methyl. In some embodiments, R^{1b} is hydrogen. In some embodiments, each R^{1a} and R^{1b} is hydrogen. In some embodiments, R^{1a} is methyl and R^{1b} is hydrogen.

In some embodiments, R^{2a} is hydrogen. In some embodiments, R^{2b} is hydrogen. In some embodiments, R², is hydrogen. In some embodiments, R^{2c} is dialkylamino. In some embodiments, R², is diethylamino. In some embodiments, R^{2d} is hydrogen. In some embodiments, each R^{2a}, R^{2b}, R^{2c}, and R^{2d} is hydrogen. In some embodiments, each R^{2a}, R^{2b}, and R^{2d} is hydrogen and R^{2c} is diethylamino.

In some embodiments, the compound of formula (200) is a compound having formula (210):

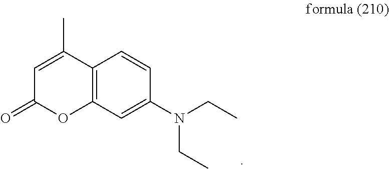 [SMILES: CCN(CC)c1ccc2c(C)cc(=O)oc2c1]

In one aspect, the disclosure provides a method of preparing a coating, the method comprising depositing a biocidal polymer solution comprising one or more polymers

### Coatings

In one aspect, the disclosure provides detectable coatings comprising one or more polymers and/or compounds of the disclosure. In a non-limiting embodiment, the coatings are grafted onto surfaces, and can be visualized using any method known in the art. In some embodiments, the detectable coating comprises one or more detectable moieties of the disclosure. In some embodiments, the detectable coating comprises one or more detectable moieties of formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (I), formula (10), formula (11), formula (12), formula (II), formula (20), formula (21), formula (VII), formula (70), formula (Iaa).

In some embodiments, the detectable coating comprises one or more polymers of the disclosure. In some embodiments, the detectable coating comprises one or more polymers comprising a moiety of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11).

In some embodiments, the detectable coating comprises one or more compounds of the disclosure. In some embodiments, the detectable coating comprises one or more compounds of formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210). In some embodiments, the detectable coating comprises one or more polymers and one or more compounds of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11), formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210). In some embodiments, the polymer is selected from PVP-silane-coalkyl-PVP and PVP-catechol-coalkyl-PVP. For non-limiting examples of PVP-silane-coalkyl-PVP, see U.S. Pat. No. 10,238,110, which is incorporated by reference herein in its entirety. In some embodiments, the coating comprises PVP-silane-coalkyl-PVP and a compound of formula (800).

In some embodiments, the coating comprises one or more compounds of formula (I), formula (II), formula (10), formula (20), formula (Ic), and/or formula (IIa) and one or more polymers in a ratio ranging from about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1.5:1 to about 1:1.5 w/w. In some embodiments, the coating comprises one or more compounds of formula (I), formula (II), formula (10), formula (20), formula (Ic), and/or formula (IIa) and one or more polymers in a ratio of about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, or about 1:9.

In some embodiments, the coating comprises PVP-silane-coalkyl-PVP and a compound of formula (IIbb), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IIbb), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (IIa), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IIa), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (800), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (800), optionally in a ratio ranging from about 2:1 to about 1:2, or about 1.5:1 to about 1:1.5 w/w, optionally in a ratio of about 1:1 w/w.

In some embodiments, the coating comprises PVP-silane-coalkyl-PVP and a compound of formula (III), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (III), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (IIIa), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IIIa), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (IIIb), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IIIb), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (300), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (300), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (301), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (301), optionally in a ratio ranging from about 2:1 to about 1:2, or about 1.5:1 to about 1:1.5 w/w, optionally in a ratio of about 1:1 w/w.

In some embodiments, the coating comprises PVP-silane-coalkyl-PVP and a compound of formula (IV), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IV), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (IVa), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IVa), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (IVb), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (IVb), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (400), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (400), optionally in a ratio ranging from about 2:1 to about 1:2, or about 1.5:1 to about 1:1.5 w/w, optionally in a ratio of about 1:1 w/w.

In some embodiments, the coating comprises PVP-silane-coalkyl-PVP and a compound of formula (VI), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (VI), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (VIa), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (VIa), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (VIb), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (VIb), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (Ic), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (Ic), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (600), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (600), optionally in a ratio ranging from about 2:1 to about 1:2, or about 1.5:1 to about 1:1.5 w/w, optionally in a ratio of about 1:1 w/w. In some embodiments, the coating comprises PVP-silane-coalkyl-PVP and a compound of formula (601), optionally in a ratio ranging from about 2:1 to about 1:2, or about 1.5:1 to about 1:1.5 w/w, optionally in a ratio of about 1:1 w/w. In some embodiments, the coating comprises PVP-catechol-coalkyl-PVP and a compound of formula (601), optionally in a ratio ranging from about 2:1 to about 1:2, or about 1.5:1 to about 1:1.5 w/w, optionally in a ratio of about 1:1 w/w.

In some embodiments, the detectable coating emits light when exposed to light of a certain wavelength. In some embodiments, the light is UV light. In some embodiments, the detectable coating emits light when exposed to light having a wavelength ranging from about 10 nm to about 400 nm.

In one aspect, the present invention provides a method of preparing a detectable coating. In some embodiments, the method comprises depositing a solution onto a surface, wherein the solution comprises one or more polymers of the disclosure and/or one or more compounds of the disclosure, and combinations thereof. In some embodiments, the solution is a biocidal polymer solution. In some embodiments, the method comprises waiting a suitable period of time for the biocidal polymer solution to dry.

In one aspect, the disclosure provides a method of preparing a detectable coating. In some embodiments, the method comprises depositing a biocidal polymer solution comprising one or more polymers of the disclosure onto a surface. In some embodiments, the method comprises waiting a suitable period of time for the biocidal polymer solution to dry. In some embodiments, the one or more polymer is selected from a polymer comprising a moiety of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11).

In one aspect, the disclosure provides a method of preparing a detectable coating. In some embodiments, the method comprises depositing a biocidal polymer solution comprising one or more compounds of the disclosure onto a surface. In some embodiments, the method comprises depositing a biocidal polymer solution comprising one or more polymers and one or more compounds of the disclosure onto a surface. In some embodiments, the method comprises waiting a suitable period of time for the biocidal polymer solution to dry. In some embodiments, the compound is cross-linked to the polymer after drying. In some embodiments, the one or more compound is a compound of formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210). In some embodiments, the polymer is selected from PVP-silane-coalkyl-PVP and PVP-catechol-coalkyl-PVP.

In some embodiments, the solution comprises an alcohol. Any alcohol can be used, as understood by one of ordinary skill in the art. Non-limiting examples of alcohols include ethanol, methanol, n-propanol, isopropanol, t-butyl alcohol, and t-amyl alcohol. In some embodiments, the solution is an antibacterial solution. In some embodiments, the solution is a biocidal solution. In some embodiments, the solution is an antiviral solution. In some embodiments, the solution is an antifungal solution. In some embodiments, the solution is an antiprotozoal solution. In some embodiments, the solution is a ready-to-use solution for grafting. In some embodiments, the solution is a biocidal polymer solution. In some embodiments, the biocidal polymer solution comprises an alcohol and one or more polymers of the disclosure. In some embodiments, the biocidal polymer solution comprises an alcohol and one or more compounds of the disclosure. In some embodiments, the biocidal polymer solution comprises an alcohol, one or more polymers, and one or more compounds of the disclosure.

In some embodiments, the biocidal polymer solution comprises a mixture of one or more polymers and one or more compounds of the disclosure. In some embodiments, the biocidal polymer solution comprises one polymer and one compound of the disclosure in a ratio of about 1:10 by weight, about 1:5 by weight, about 1:4 by weight, about 1:3 by weight, about 1:2 by weight, about 1:1 by weight, about 2:1 by weight, about 3:1 by weight, about 4:1 by weight, about 5:1 by weight, or about 10:1 by weight. In some embodiments, the biocidal polymer solution comprises one polymer and one compound of the disclosure in a ratio of about 1:1 by weight. In some embodiments, the compound a compound of any of formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210).

In some embodiments, the biocidal polymer solution comprises one or more polymers of the disclosure. In some embodiments, the one or more polymers are present in the biocidal polymer solution at a concentration ranging from about 1 mg/mL to about 100 mg/mL, about 5 mg/mL to about 50 mg/mL, about 5 mg/mL to about 25 mg/mL or about 8 mg/mL to about 12 mg/mL. In some embodiments, the one or more polymers are present in the biocidal polymer solution at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some embodiments, the method comprises waiting a suitable period of time for the biocidal polymer solution to dry, as would be understood by one of ordinary skill in the art. In some embodiments, a suitable period of time for the biocidal polymer solution to dry ranges from about 1 minute to about 12 hours, about 1 minute to about 15 minutes, about 5 minutes to about 10 minutes, about 6 hours to about 12 hours, or about 3 hours to about 6 hours. In some embodiments, the drying is performed at a temperature in a range from about 0° C. to about 250° C., about 25° C. to about 100° C., about 100° C. to about 120° C., about 30° C. to about 75° C. or about 20° C. to about 30° C. In some embodiments, the drying is performed at room temperature, above room temperature, about 20° C., about 25° C., about 30° C., about 50° C., about 75° C., about 100° C., about 120° C., about 125° C., about 150° C., about 175° C., or about 200° C.

The coatings described herein may be applied to any surface. Non-limiting examples of surfaces include metals such as chromium, iron, cobalt, cobalt-chrome alloys, aluminum, titanium and titanium alloys, iron, and steel, such as stainless steel; metal oxides; ceramics; polymers such as polyethylene, Teflon, polyethylene terephthalate, and polypropylene, silicones, rubbers, latex, plastics, polyanhydrides, polyesters, polyorthoesters, polyamides, polyacrylonitrile, polyurethanes, polytetrafluoroethylene, polyethylenetetraphthalate and polyphazenes, leather, textiles or textile materials, synthetic fabrics such as nylon and polyester; textile material comprising fibers comprising fiber material such as acrylic polymers, acrylate polymers, aramid polymers, nylon, polyolefins, polyester, polyamide, polypropylene, rayon, spandex, silk, viscose, silicon, and glass. In a non-limiting embodiment, the durability of the coatings was found to be particularly efficacious when the compositions and formulations were coated onto metallic substrates. In some embodiments, the surface is a metallic surface. In some embodiments, the surface is activated and/or naturally hydroxylated.

### Grafting

In one aspect of the disclosure, methods for grafting graftable substrates of the disclosure are provided.

In one aspect of the disclosure, methods for grafting polymers are provided. In some embodiments, the polymer is one or more polymers comprising one or more polymers comprising a moiety of formula (I), formula (10), formula (11), formula (12), formula (II), formula (20), formula (21), formula (VII), formula (70), formula (Iaa), formula (Iaa), formula (Ia), formula (IIaa), formula (IIa), formula (VIIa), formula (VIIb), formula (Ibb), formula (Ib), formula (IXa), formula (IXb), formula (IXc), formula (IXa1), formula (IXb1), formula (IXc1), formula (IXa2), formula (IXb2), formula (IXc2), formula (IXe), formula (IXf), formula (IXg), formula (IXh), formula (3), formula (4), formula (5), formula (6), formula (7), formula (8), formula (9), and/or formula (11).

In one aspect of the disclosure, methods for grafting compounds are provided. In some embodiments, the compound is a compound of any one of formula (III), formula (IIIa), formula (IIIb), formula (300), formula (301), formula (IV), formula (IVa), formula (IVb), formula (400), formula (VI), formula (VIa), formula (VIb), formula (Ic), formula (600), formula (601), formula (VIII), formula (VIIIa), formula (800), formula (100), formula (200), and/or formula (210).

In one aspect, the disclosure describes methods for grafting a substrate onto a surface. In some embodiments, the method includes depositing a graftable substrate of the disclosure onto the surface; and heating the surface for a period of time. In some embodiments, the graftable substrate is deposited by spraying, dip coating, or spin-coating. In some embodiments, the graftable substrate is deposited in a solvent comprising an alcohol selected from ethanol, methanol, n-propanol, isopropanol, t-butyl alcohol, and t-amyl alcohol.

In one aspect, the disclosure describes methods of grafting a polymer onto a surface, the method comprising depositing a polymer of the disclosure onto the surface; and heating the surface for a period of time. In some embodiments, the polymer is deposited in a solvent. In some embodiments, the solvent is an alcohol. In some embodiments, the solvent is water. Non-limiting examples of alcohols include ethanol, methanol, n-propanol, isopropanol, t-butyl alcohol, and t-amyl alcohol.

In another aspect, the disclosure describes methods of grafting a compound onto a surface, the method comprising depositing a compound of the disclosure onto the surface; and heating the surface for a period of time. In some embodiments, the compound is deposited in a solvent. In some embodiments, the solvent is an alcohol. In some embodiments, the solvent is water. Non-limiting examples of alcohols include ethanol, methanol, n-propanol, isopropanol, t-butyl alcohol, and t-amyl alcohol.

Any method of deposition is contemplated for use herein, as would be understood by one of ordinary skill in the art. Non-limiting examples of methods of deposition include spraying, dip coating, or spin-coating.

Any temperature suitable for grafting is contemplated for use herein, as would be understood by one of ordinary skill in the art. In some embodiments, the surface is heated to a temperature ranging from about 50° C. to about 120° C. In some embodiments, the surface is heated to a temperature of about 110° C.

As would be understood by one of ordinary skill in the art, any temperature suitable for grafting is contemplated for use herein. In some embodiments, the surface is heated for a period of time ranging from about 15 minutes to about 12 hours. In some embodiments, the surface is heated for a period of time ranging from about 6 hours to about 12 hours. In some embodiments, the surface is heated for a period of time ranging from about 15 minutes to about 90 minutes. In some embodiments, the surface is heated for a period of time ranging from about 30 minutes to about 60 minutes. In some embodiments, if the polymer or compounds to be grafted comprises a catechol moiety, the surface is heated for a period of time ranging from about 15 minutes to about 12 hours.

In some embodiments, the method further comprises washing the surface with a solvent. Non-limiting examples of solvents that can be used for washing include ether.

In some embodiments, the method further comprises sonicating the surface. Sonication can be performed for periods of time including, but not limited to, 5 minutes, 15 minutes, or 30 minutes. In some embodiments, the surface is sonicated while in a solvent. In non-limiting examples, solvents useful for sonication include acetone, ethanol, and distilled water.

In some embodiments, the surface is activated prior to grafting. Non-limiting examples of activation include plasma activation, acid activation, or UV/ozone activation.

### Methods of Use

In one aspect of the disclosure, methods for controlling the growth of at least one bacteria, fungi, protozoa, or virus are provided. In some embodiments, the method comprises grafting one or more polymers of the disclosure and/or one or more compounds of the disclosure onto a surface. In some embodiments, the method comprises grafting one or more polymers and one or more compounds of the disclosure. In some embodiments, the surface is activated prior to grafting.

In some embodiments, the bacteria is a gram-positive bacteria selected from M. tuberculosis (including multi drug resistant TB and extensively drug resistant TB), M. bovis, M. typhimurium, M. bovis strain BCG, BCG substrains, Mavium, Mintracellulare, Mafricanum, M. kansasii, M. marinum, Mulcerans, Mavium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthraces, B. subtilis, Nocardia asteroides, and other Nocardia species, Streptococcus viridans group, Peptococcus species, Peptostreptococcus species, Actinomyces israelii and other Actinomyces species, Propionibacterium acnes, Clostridium tetani, Clostridium perfringens, Clostridium botulinum, other Clostridium species, and Enterococcus species.

In some embodiments, the bacteria is a gram-negative bacteria selected from Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species, Brucella abortus, other Brucella species, Chlamydia trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, Escherichia hirae, and other Escherichia species, as well as other Enterobacteriacae, Burkholderia cepacia, Burkholderia pseudomallei, Francisella tularensis, Bacteroides fragilis, Fusobascterium nucleatum, Provetella species, Cowdria ruminantium, Klebsiella species, and Proteus species.

In some embodiments, the virus is selected from avian influenza, human immunodeficiency virus, herpex simplex virus, human respiratory syncytial virus, Middle East respiratory syndrome-related coronavirus (MERS-CoV), rhinovirus, polio, rotavirus, measles, Ebola, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, severe acute respiratory syndrome (SARS), severe acute respiratory syndrome coronavirus 2 (SARS-CoV-2), parainfluenza, and Pichinde viruses.

In another aspect, the disclosure describes a method of preventing digestion of cellulose by an organism. In some embodiments, the method comprising grafting a polymer or a compound of the disclosure onto a surface comprising cellulose. In some embodiments, surface comprises wood cellulose. In some embodiments, the organism is selected from a wood boring gribble, a shipworm, a woodlice, and a wood-boring insect. Non-limiting examples of wood-boring insects include termites, bark beetles, horntail larvae, moth larvae, beetles. In some embodiments, the insect is a xylophage. Non-limiting examples of xylophages include termites, bark beetles, horntail larvae, moth larvae, and beetles.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Dye-Labelled Graftable PVP-based Biocidal Polymer

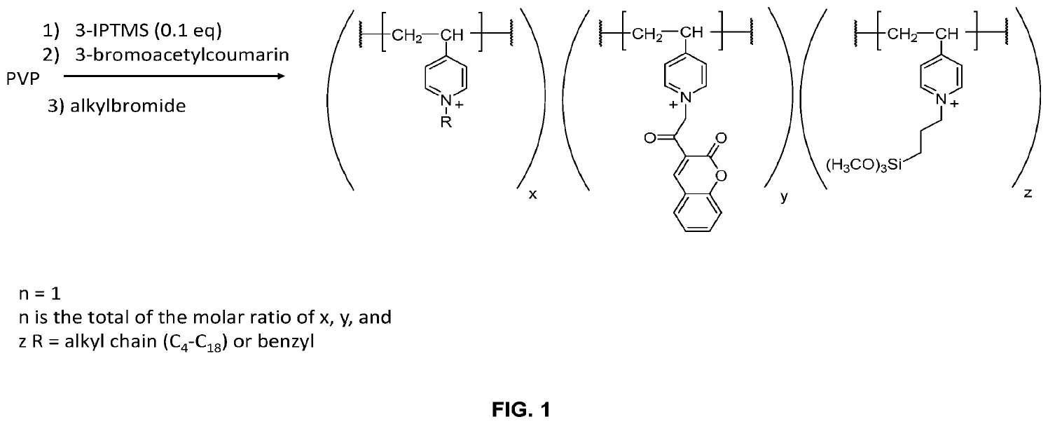

This Example describes a method for identifying the presence of an aminated polymer-based grafted or coated layer by covalently attaching a fluorescent marker to a biocidal polymer using polyvinylpyridine (PVP), as described in FIG. 1. The molar ratio of each moiety x, y, and z is as follows:

$$n = x + y + z = 1$$

$$x = 0.6 - 0.8 ,$$

$$y = 0.1 - 0.2 ; and$$

$$z = 0.1 - 0.2 .$$

The random copolymer is synthesized by quaternization of polyvinylpyridine (PVP) (Mw=60,000) or methyl-polyethyleneimine (PEI) first with halogenoacetylcoumarin then with a halogenopropyltrimethoxysilane (halogen: Br, I, or Cl). In a non-limiting example, 3-bromoacetylcoumarin is used since it is a commercially available compound (Sigma-Aldrich catalogue). The advantage of using 3-bromoacetylcoumarin, besides its availability, is its low cost in the current market when compared to other fluorescent dyes which are reactive to UV light. Any other fluorescent dyes could be used as needed for specific purposes based on the chemical scheme, as would be understood by one of ordinary skill in the art.

The quaternization is performed in refluxing ethanol/methanol for 3 to 6 hours with a mixture of halogenoacetylcoumarin and a halogenotrialkoxysilane. Then, a bromoalkyl chain or benzylbromide is added to complete the quaternization for another period (24 h to 96 h).

Such grafted or coated polymer may be visible to the naked eye by shining the layer with UV light (365 nm) which reflects green, blue or yellow fluorescent light.

### Experimental Protocol:

PVP (3 g) was dissolved in methanol (300 mL) in a round-bottom flask. After complete dissolution of PVP, 3-iodopropyltrimethoxysilane (350 microliters) along with 3-bromoacetylcoumarin (400 mg) were added. The mixture was stirred under reflux for 6 hours at 50° C. An alkylbromide (1-bromodecane, 30 mL) was added to the solution. The solution was kept under reflux for 48 hours. FTIR-ATR (diamond): 3066 CH═CH, 2933 CH₂, 2857 CH₂, 1731 C═O (coumarin), 1648 ν C—N⁺, 1604 C═C (coumarin), 1472 ν C—N⁺, 1173 (Si—O-Me) cm⁻¹.

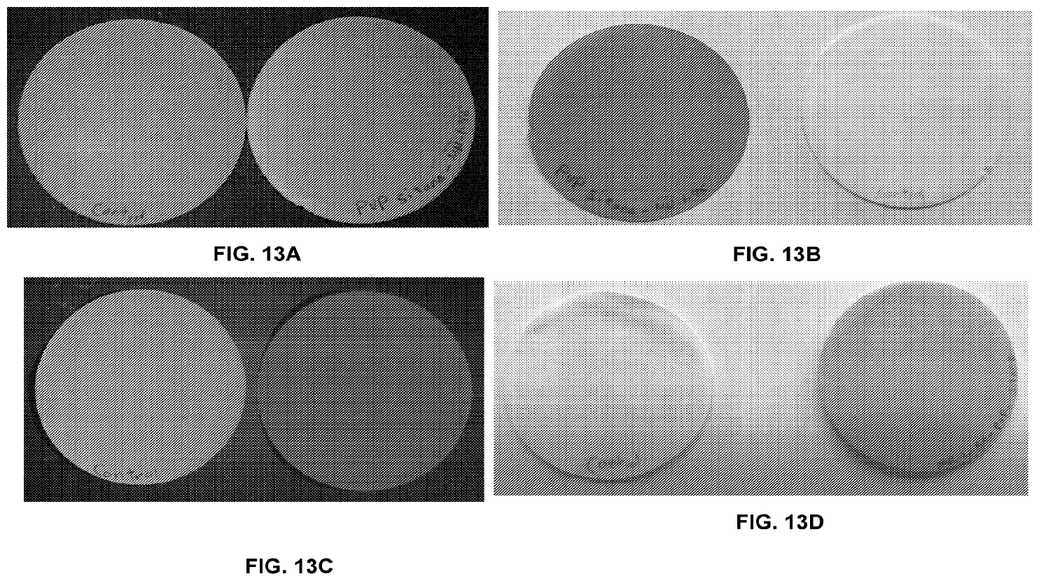

A filter paper was impregnated with the fluorescent catechol solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange. Filter papers under uv light photos were taken after drying for 10 minutes at 100° C. FIGS. 13A and 13B show images of a comparison of filter paper samples under UV light (FIG. 13A and FIG. 13C) and fluorescein test (FIG. 13B and FIG. 13D).

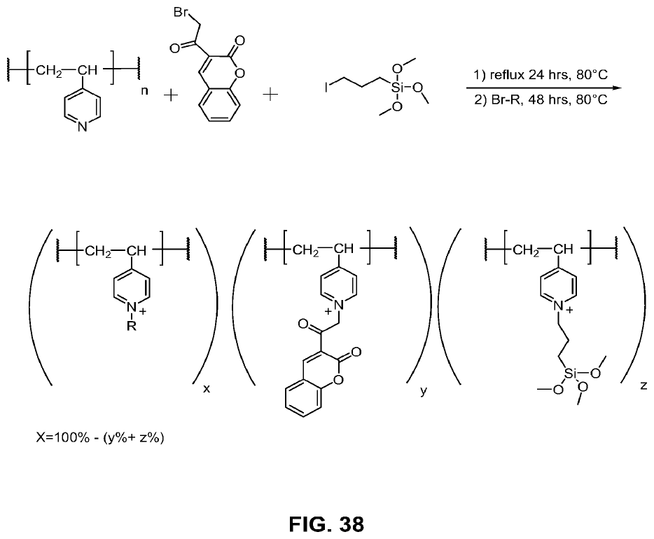

An exemplary reaction scheme is shown in FIG. 38. In some embodiments, 3-(bromoacetyl) coumarin can be replaced with 2-oxo-2H-chromen-7-yl 2-bromoacetate:

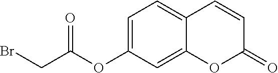 [SMILES: O=C(CBr)Oc1ccc2ccc(=O)oc2c1]

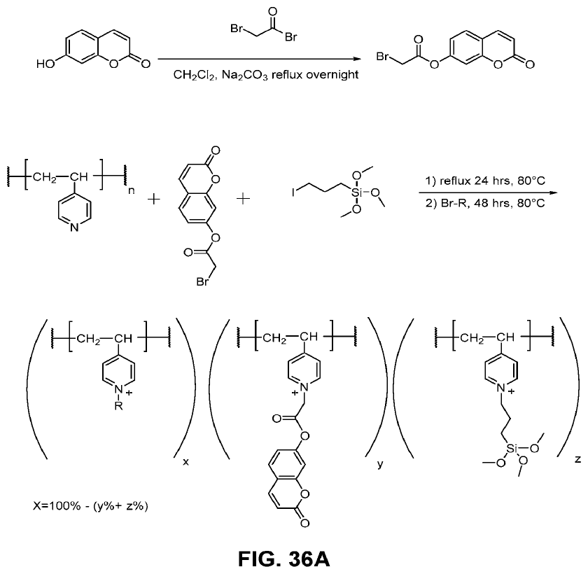

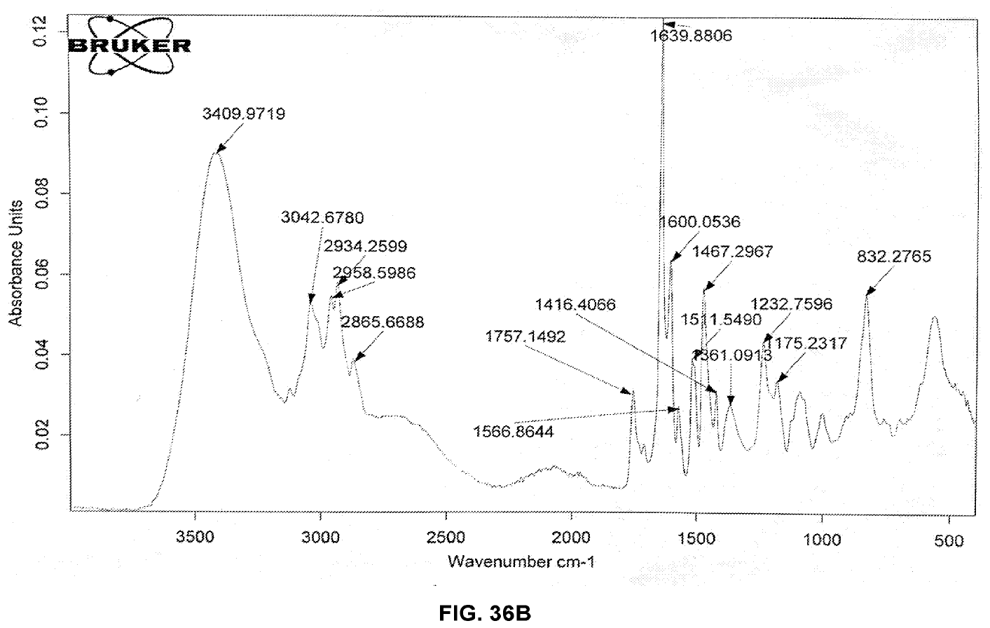

(see FIG. 36A as an non-limiting example).

In a non-limiting example, polyvinylpyridine (0.5 g, Mn=60,000), was dissolved in methanol (30 mL), -oxo-2H-chromen-7-yl 2-bromoacetate (132 mg, 0.465 mmol) and 3-iodopropyltrimethoxysilane (93 μL, 0.465 mmol) were added in 10% stoichiometric ratio, and the and the mixture was stirred under reflux during overnight at 70° C. 1-bromobutane (1.3 mL, 9.3 mmol) in 10 mL of methanol was added to the reaction solution. The reaction was allowed to stir for two days at 70° C. FTIR-ATR (diamond): 3042 CH═CH, 2934 CH₂, 2865 CH₂, 1757 C═O (coumarin), 1639 ν C═N⁺, 1600 C═C (coumarin), 1175 (Si—O-Me) cm⁻¹. FIG. 36B shows the IR spectrum.

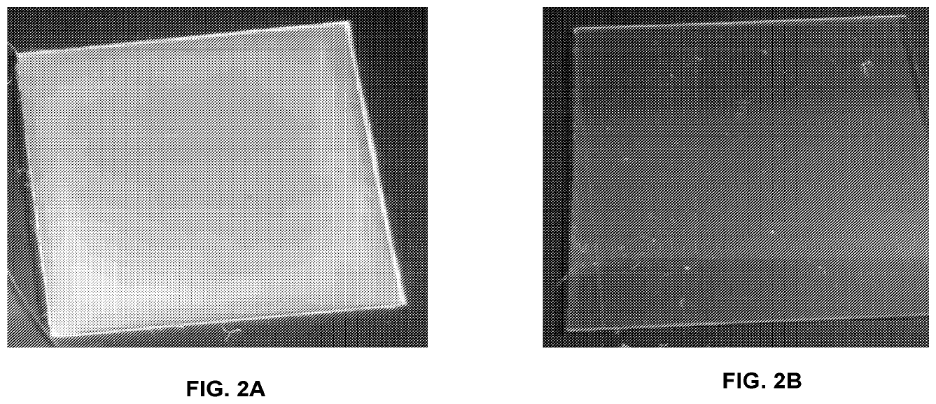

An activated glass slide was immersed in the prepared polymer solution (10 mg/mL). The solution was then evaporated to dryness in an oven at 120° C. The unbound copolymer was then removed by extensive washing with methanol, and then air-dried. The glass slide was observed under UV light (365 nm) and visually compared with a control. The treated slide (FIG. 2A) exhibited markedly yellow fluorescence while the control (FIG. 2B) did not shine under UV light.

### Example 2: Use of Fluorescent Silane as a Guest in a Biocidal Polymer Solution

This Example describes a method for identifying the presence of an aminated polymer-based grafted or coated layer by introducing a UV-active guest molecule into the prepared biocidal polymer solution used to prepare the layer. During curing, a covalent bond will form between the guest molecule and the biocidal polymer (cross-linking). This method can be used with coatings as well as surface grafting or modification.

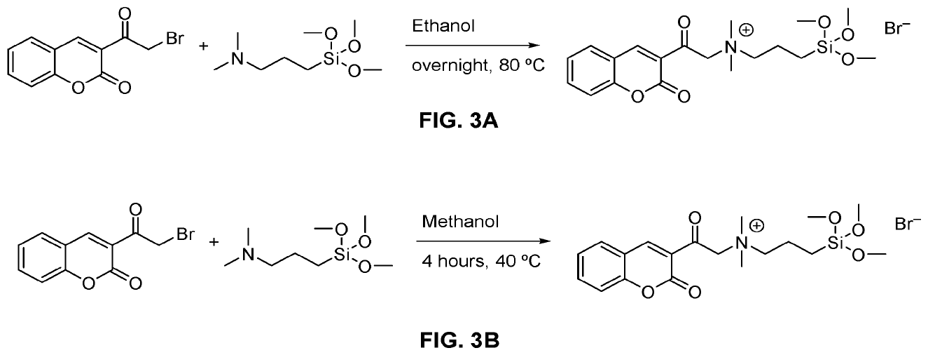

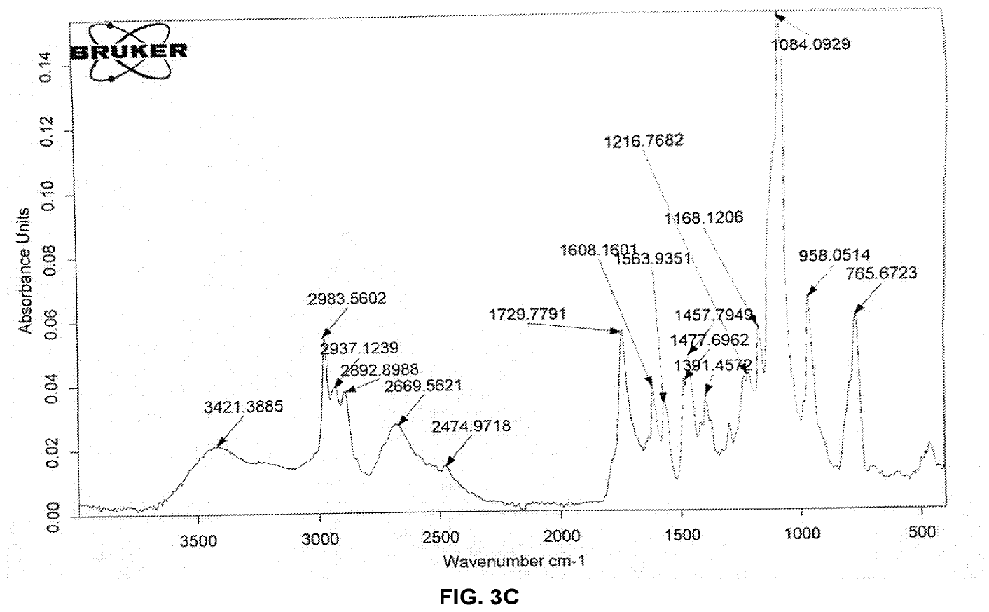

The fluorescent guest molecule was obtained by quaternization of N—N dimethylpropyltrimethoxysilane with 3-bromoacetylcoumarin (FIGS. 3A and 3B).

### Experimental Protocol:

In a non-limiting example, 3-bromoacetylcoumarin (275 mg, 1 mmol), and 3 N,N-dimethylaminopropyltrimethoxysilane (218 μL, 1 mmol) (FIG. 3A) were dissolved in 30 mL of ethanol and the mixture was stirred under reflux overnight at 80° C. FTIR-ATR (diamond): cm−1. The mixture was stirred for overnight at 80° C. FTIR-ATR (diamond): 2983 CH₂, 2937 CH₂, 2892 CH₂, 2669 CH₃, 1729 C═O (coumarin), 1608 C═C (coumarin), 1477 ν C—N⁺, 1084 (Si—O-Me) cm⁻¹. FIG. 3C shows the IR spectrum.

In a non-limiting example, 3-bromoacetylcoumarin (1 eq.) in methanol (40 mL) was treated with 3 N,N dimethylaminopropyltrimethoxysilane (FIG. 3B). The mixture was stirred for 4 hours at 40° C. The resultant quaternary ammonium compound was obtained with a high yield.

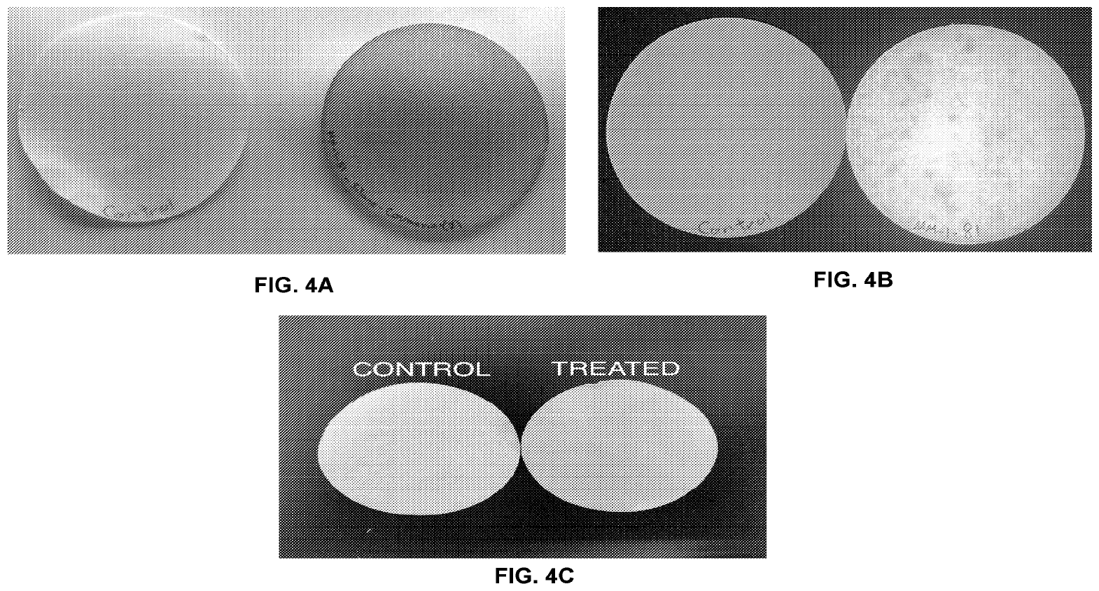

A filter paper was impregnated with the fluorescent silane solution and dried at 100° C. for 10 minutes. A fluorescein test was performed on both a control filter paper and the treated filter paper sample (FIGS. 4A and 4C). The control filter paper was prepared by impregnating filter paper with ethanol and drying for 10 minutes at 100° C. In contrast to the control which appeared white, the filter paper treated with the fluorescent silane solution appeared yellow, demonstrating that the fluorescent silane was effectively grafted onto the filter paper. Filter papers under uv light photos were taken after drying for 10 minutes at 100° C. (FIG. 13A) and fluorescein test (FIG. 4B).

Following verification of the grafting ability of the fluorescent silane, a solution comprising a mixture of PVP-silane-coalkyl-PVP (made according to the method described in U.S. Pat. No. 10,238,110, which is incorporated by reference herein in its entirety) and the fluorescent silane in methanol in the ratio 1:1 w/w was prepared.

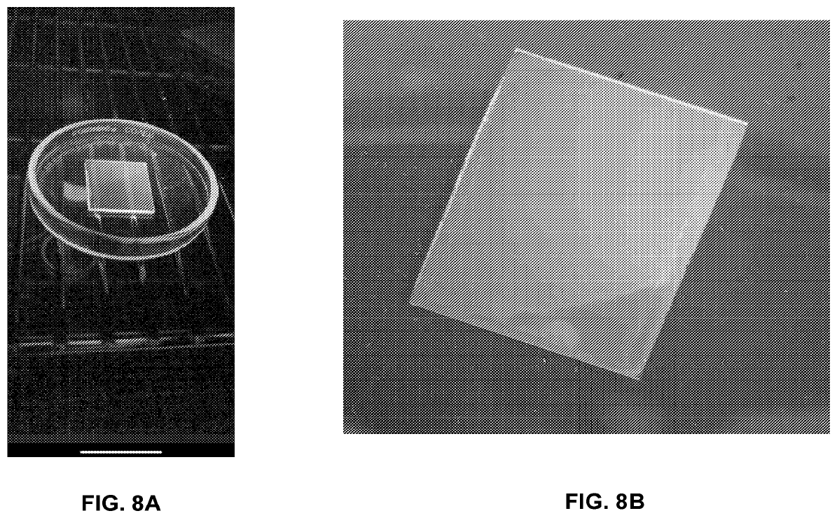

An activated glass slide was immersed in the prepared polymer solution (10 mg/mL). The solution was then evaporated to dryness in an oven at 120° C. The unbound copolymer was then removed by extensive washing with methanol and air-dried. The glass slide was observed under UV light (365 nm) and visually compared with a control. As shown in FIGS. 8A and 8B, the slide coated with the prepared polymer solution exhibited markedly green fluorescence under UV light.

### Example 3: One-Pot Synthesis and Deposition of Graftable Fluorescent Biocidal Polymers (Catechol-Based)

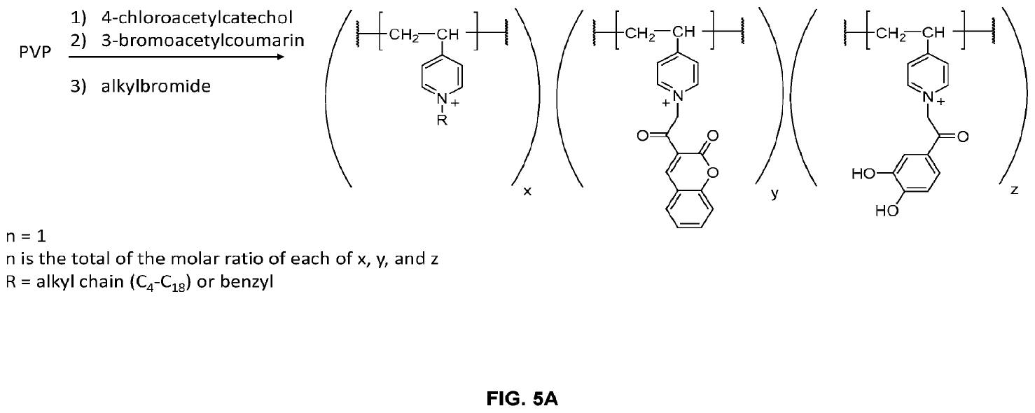

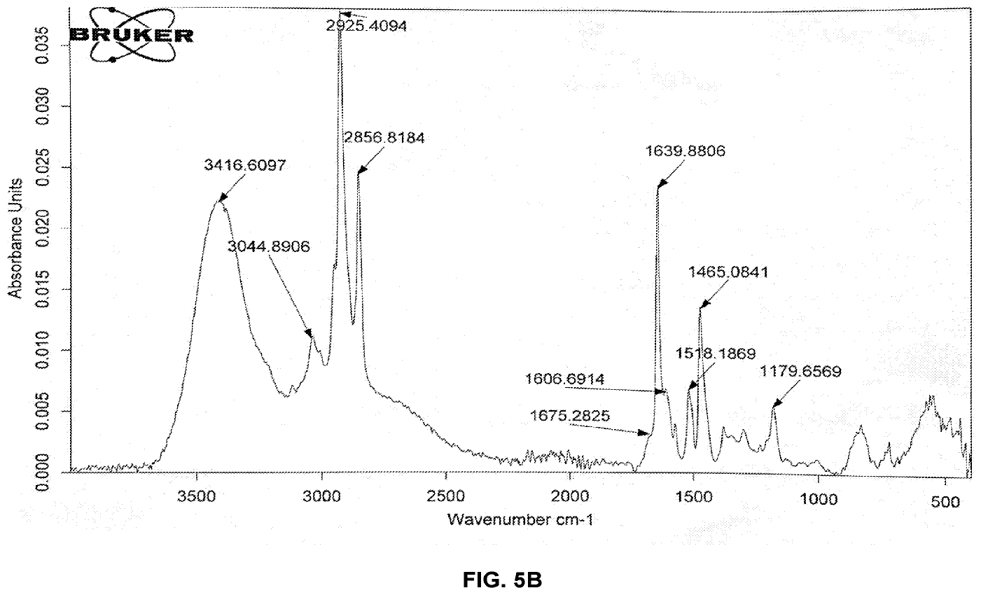

This Example describes a method for identifying the presence of a grafted or coated layer by covalently attaching a fluorescent marker to a biocidal polymer using polyvinylpyridine (PVP), as described in FIG. 5. The molar ratio of each moiety x, y, and z is as follows:

$$n = x + y + z = 1$$

$$x = 0.6 - 0.8 ,$$

$$y = 0.1 - 0.2 ; and$$

$$z = 0.1 - 0.2 .$$

The random copolymer is synthesized by quaternization of PVP (Mw=60,000) or methyl-PEI first with halogenoacetylcoumarin then with a halogenoacetylcatechol (halogen: Br, I, or Cl). In a non-limiting example, 3-bromoacetylcoumarin is used since it is a commercially available compound (Sigma-Aldrich catalogue). In some embodiments, the advantage of using 3-bromoacetylcoumarin, besides its availability, is its low cost in the current market when compared to other fluorescent dyes which are reactive to UV light. In a non-limiting example, any other fluorescent dyes could be used as needed for specific purposes based on the same chemical scheme, as would be understood by one of ordinary skill in the art.

The quaternization is performed in refluxing ethanol/methanol for 3 to 6 hours with a mixture of halogenoacetylcoumarin and a halogenoacetylcatechol. Then, a bromoalkyl chain or benzylbromide is added to complete the quaternization for another period (24 h to 96 h).

The grafted or coated polymer may be visible to the naked eye by irradiating the layer with UV light (365 nm) which reflects yellow fluorescent light.

### Experimental Protocol:

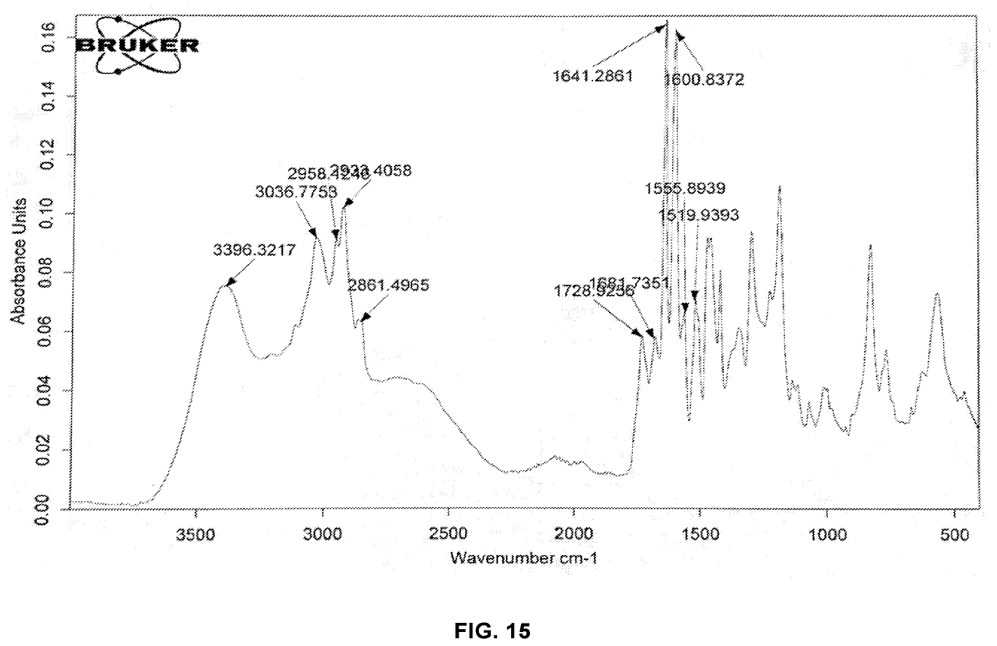

In a non-limiting example, polyvinylpyridine (1 g, Mn=60,000), was dissolved in methanol (70 mL), 3-(bromoacetyl) coumarin (250 mg, 0.936 mmol) and 2-chloro-3,4-dihydroxy-acetophenone (87 mg, 0.465 mmol) were added in 10% stoichiometric ratio, and the and the mixture was stirred under reflux overnight at 70° C. 1-bromobutane (1.3 mL, 9.3 mmol) in 10 mL of methanol was added to the reaction solution. The reaction was allowed to stir for two days at 70° C. FTIR-ATR (diamond): 3404 OH (catechol), 3047 CH═C (coumarin), 2959 CH═CH, 2929 CH₂, 2866 CH₂, 1732 C═O (coumarin), 1683 C═O (coumarin), 1640 ν C—N⁺, 1602 (C═C), 1472 νC—N⁺, 1298 C═C (catechol) cm¹. FIG. 15 shows an image of the IR spectrum.

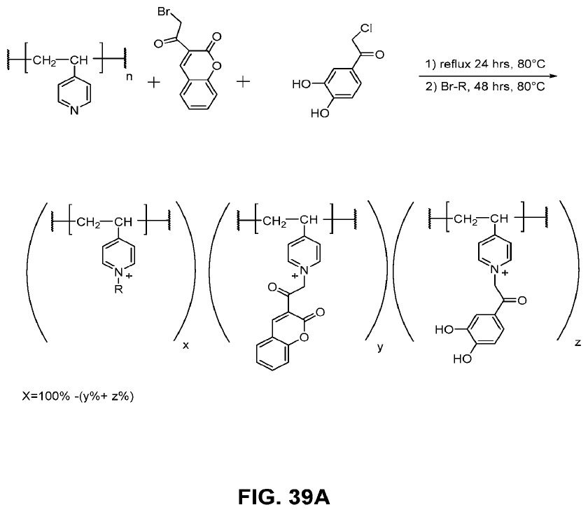

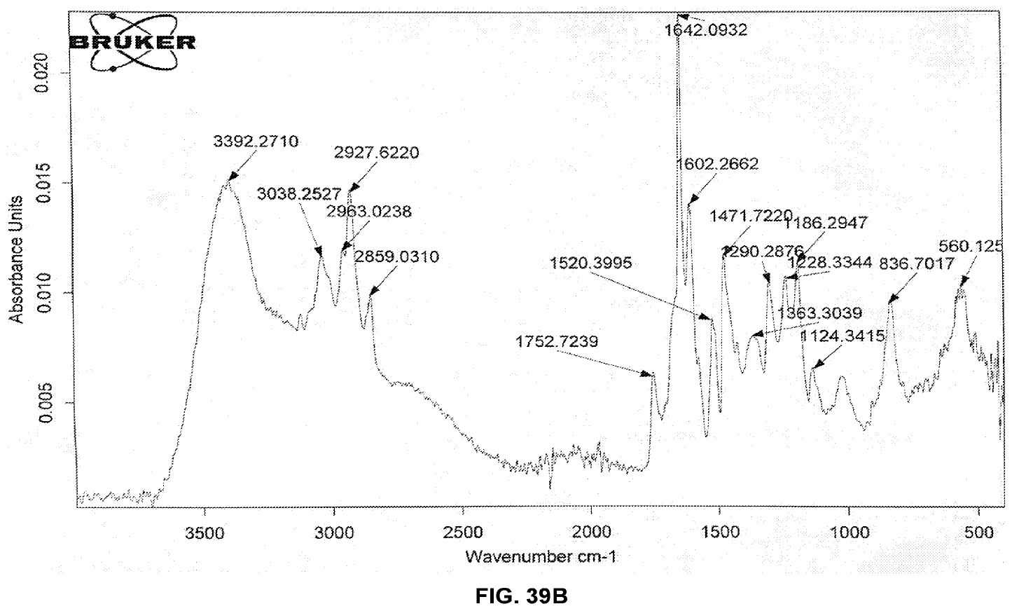

An exemplary reaction scheme is shown in FIG. 39A. In some embodiments, 3-(bromoacetyl) coumarin can be replaced with 2-oxo-2H-chromen-7-yl 2-bromoacetate:

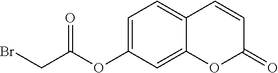 [SMILES: O=C(CBr)Oc1ccc2ccc(=O)oc2c1]

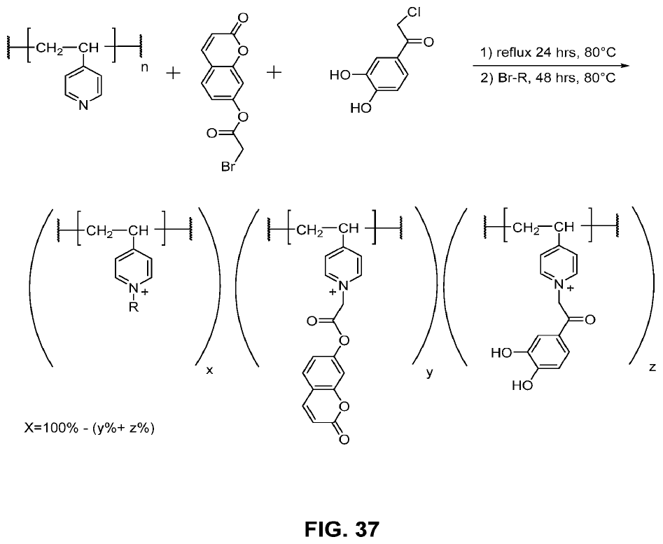

(see FIG. 37 as an non-limiting example).

In a non-limiting example, polyvinylpyridine (0.5 g, Mn=60,000), was dissolved in methanol (30 mL), -oxo-2H-chromen-7-yl 2-bromoacetate (132 mg, 0.465 mmol) and 2-chloro-3,4-dihydroxy-acetophenone (250 mg, 0.931 mmol) were added in 10% stoichiometric ratio, and the and the mixture was stirred under reflux during overnight at 70° C. 1-bromobutane (1.3 mL, 9.3 mmol) in 10 mL of methanol was added to the reaction solution. The reaction was allowed to stir for two days at 70° C. FTIR-ATR (diamond): 3392 OH (catechol), 3038 CH═C (coumarin), 2927 CH₂, 2860 CH₂, 1752 ν C═O (coumarin), 1642 ν C═N+, 1602 ν (C═C) cm¹. FIG. 39B shows the IR spectrum.

In a non-limiting example, PVP (1 Eq.) is dissolved in methanol in a round-bottom flask. After complete dissolution of PVP, 4-chloroacetylcatechol (0.05 to 0.2 Eq) along with 3-bromoacetylcoumarin (0.1 Eq) are added. The mixture is stirred under reflux for 6 hours at 50° C.

A slight excess of alkylbromide or benzyl-bromide added to the solution. The solution is stirred under reflux for 48 hours.

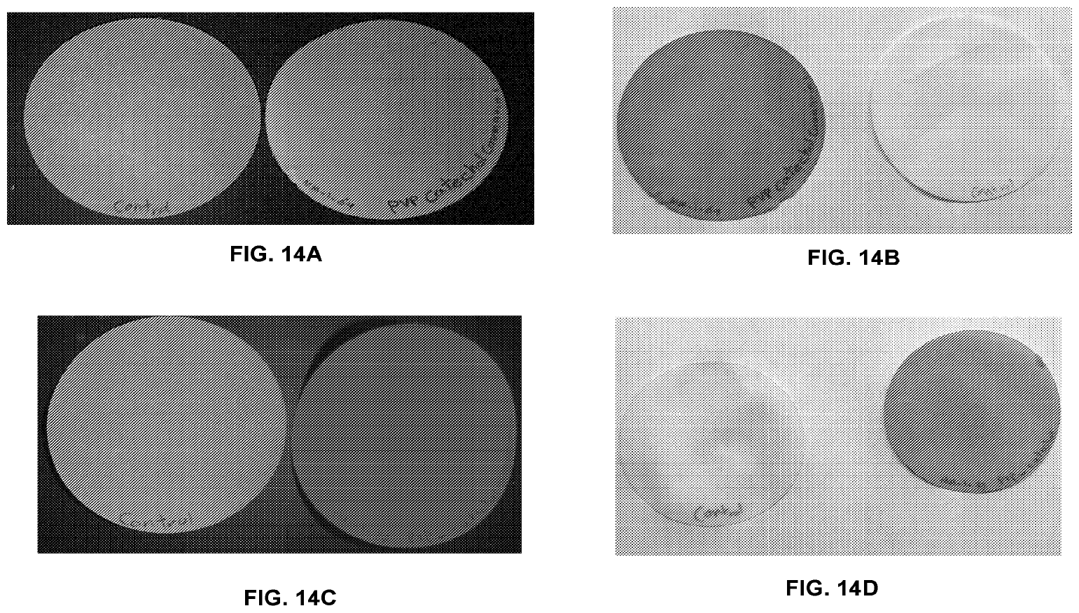

A filter paper was impregnated with the fluorescent catechol solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange. Treated filter was observed under UV light (365 nm) and visually compared with a control. The treated filter exhibited markedly yellow fluorescence while the control did not shine under UV light. FIGS. 14A-14D show images of a comparison of filter paper samples under UV light (FIG. 14A and FIG. 14C) and fluorescein test (FIG. 14B and FIG. 14D).

An activated glass slide is immersed in the prepared polymer solution. The solution is then evaporated to dryness in an oven at 120° C. The unbound copolymer is then removed by extensive washing with methanol and air-dried. The glass slide is observed under UV light (365 nm) and visually compared with a control. The treated slide exhibits markedly green fluorescence while the control does not shine under UV light.

### Example 4: Use of Fluorescent Catechol as a Guest in a Biocidal Polymer Solution

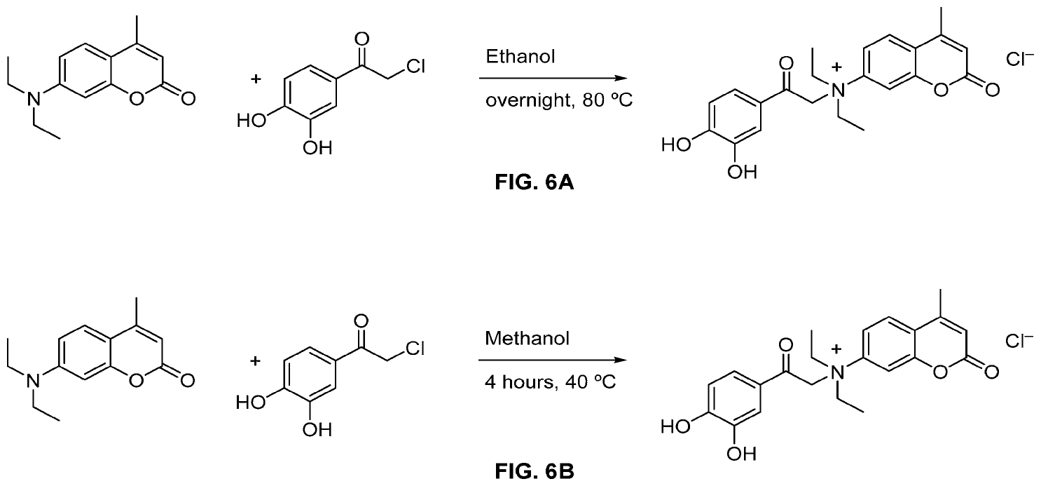

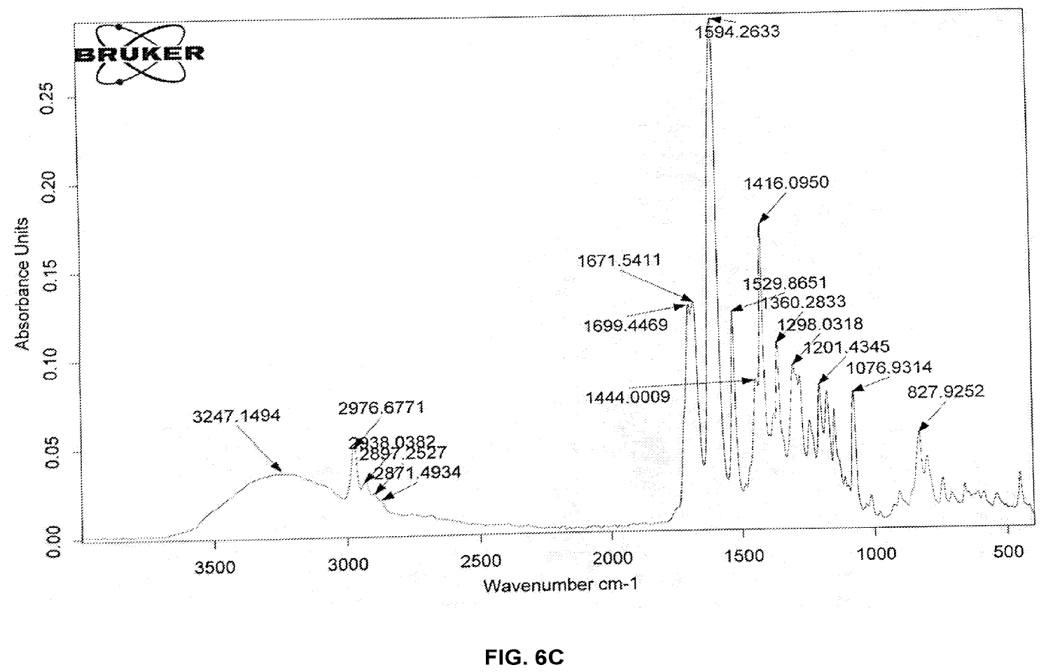

This Example describes a method for introducing a guest molecule into a prepared biocidal polymer solution used to form a grafted or coating layer. After deposition, both the guest molecule and the biocidal polymer form a covalent bond with the activated surface. In a non-limiting example, this method is particularly useful for covalent grafting or surface modifications on metals (non-limiting examples of metals include Cr, Fe, Ti, Co, stainless steel and other alloys). The fluorescent guest molecule was obtained by quaternization of 4-halogenoacetylcatechol (for example the commercial 4-chloroacetylcatechol) with a dialkylaminocoumarin (R=—CH₃ or —C₂H₅) such as the commercially available 7-diethylamino-4-methylcoumarin (FIGS. 6A and 6B).

In a non-limiting example, as depicted in FIG. 6A, 7-diethylamino-4-methylcoumarin (231 mg, 1 mmol), and 2-chloro-3,4-dihydroxy acetophenone (186 mg, 1 mmol) were dissolved in 40 mL of ethanol. The reaction was allowed to stir for 12 hours at 80° C. FTIR-ATR (diamond): 2975 CH₂, 2930 CH₂, 2886 CH₂, 1725 C═O (coumarin), 1601C═C (coumarin), 1420 ν C—N⁺, 1077 (Si—O-Me) cm⁻¹. FIG. 6C shows an image of the IR spectrum.

In a non-limiting example, as depicted in FIG. 6B, 7-diethylamino-4-methylcoumarin (1 Eq) in methanol (40 mL) is treated with 4-chloroacetylcatechol. The mixture is stirred for 4 hours at 40° C. The resultant quaternary ammonium compound is obtained with a high yield.

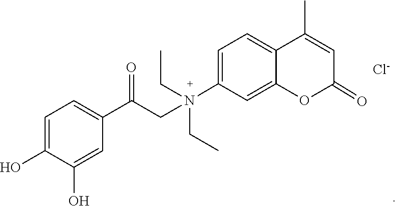

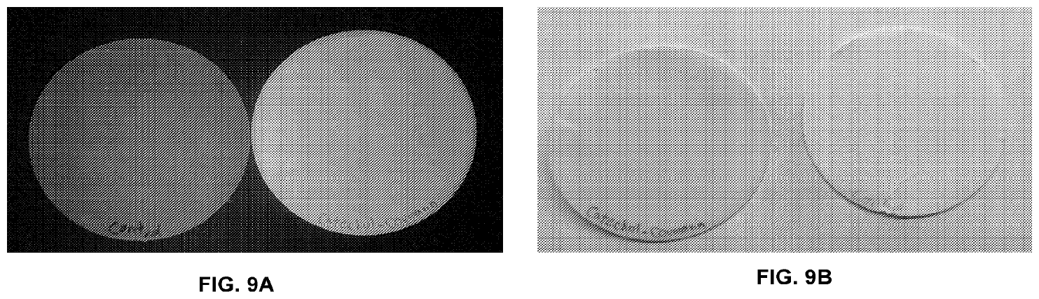

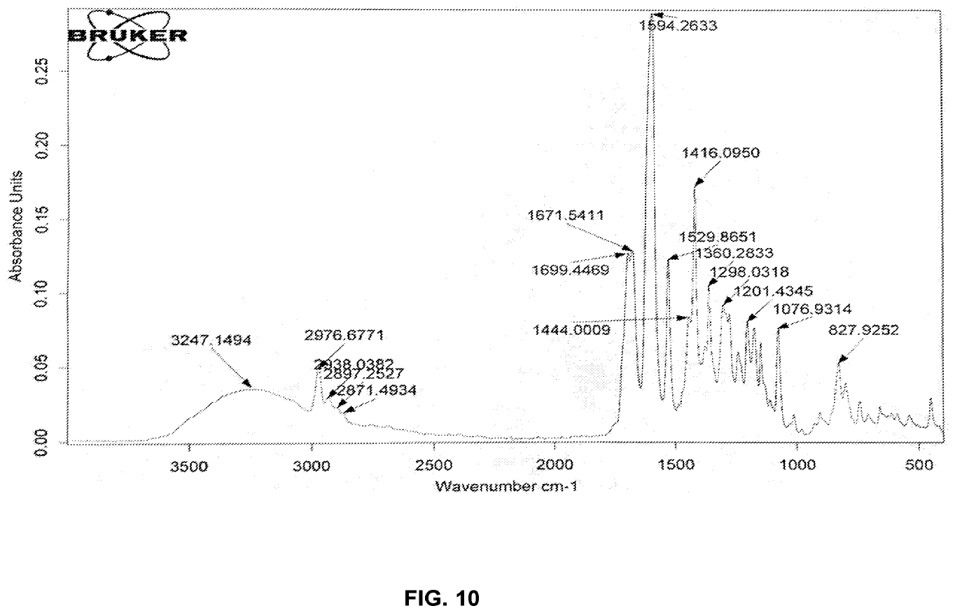

A filter paper was impregnated with the fluorescent catechol solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent catechol is effectively grafted on filter paper which appears yellow. FIGS. 9A and 9B show images of a comparison of filter paper samples under UV light (FIG. 9A) and fluorescein test (FIG. 9B).

Following verification of the grafting ability of the fluorescent catechol, a solution comprising a mixture of PVP-catechol-coalkyl-PVP and the guest fluorescent catechol compound in the ratio 1:1 w/w is prepared. In a non-limiting example, the PVP-catechol-coalkyl-PVP is prepared according to the following scheme:

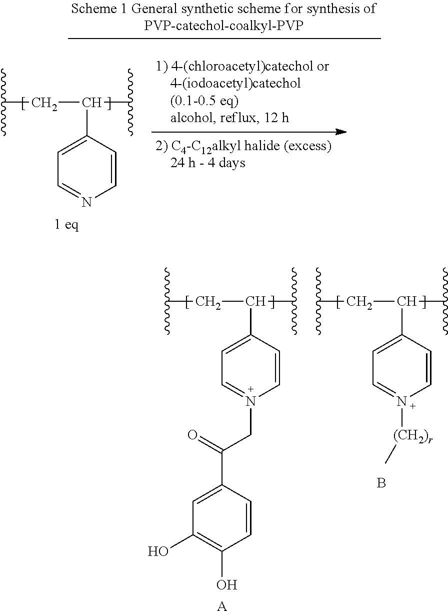

In a non-limiting example, polyvinylpyridine (1 g, Mn=60,000), was dissolved in methanol (70 mL). 2-chloro-3,4-dihydroxy-acetophenone (87 mg, 0.465 mmol) was added in 10% stoichiometric ratio, and the and the mixture was stirred under reflux during overnight at 70° C. 1-bromobutane (1.3 mL, 9.3 mmol) in 10 mL of methanol was added to the reaction solution. The reaction was allowed to stir for two days at 70° C. FTIR-ATR (diamond): 3416 OH (catechol), 3044 CH═C, 2925 CH═CH, 2929 CH₂, 2856 CH₂, 1675 C═O, 1640 ν C═N⁺, 1602 (C═C) cm⁻¹.

An activated glass slide is immersed in the prepared polymer solution (10 mg/mL). The solution is then evaporated to dryness in an oven at 120° C. The control glass slide is prepared by immersing the slide in methanol, drying at 120° C., and then observed under UV light. The unbound copolymer is then removed by extensive washing with methanol and air-dried. The glass slide is observed under UV light (365 nm) and visually compared with a control.

### Example 5: One-Pot Synthesis and Deposition of Graftable Fluorescent Biocidal Non-Aminated Polymers (Silane-Based)

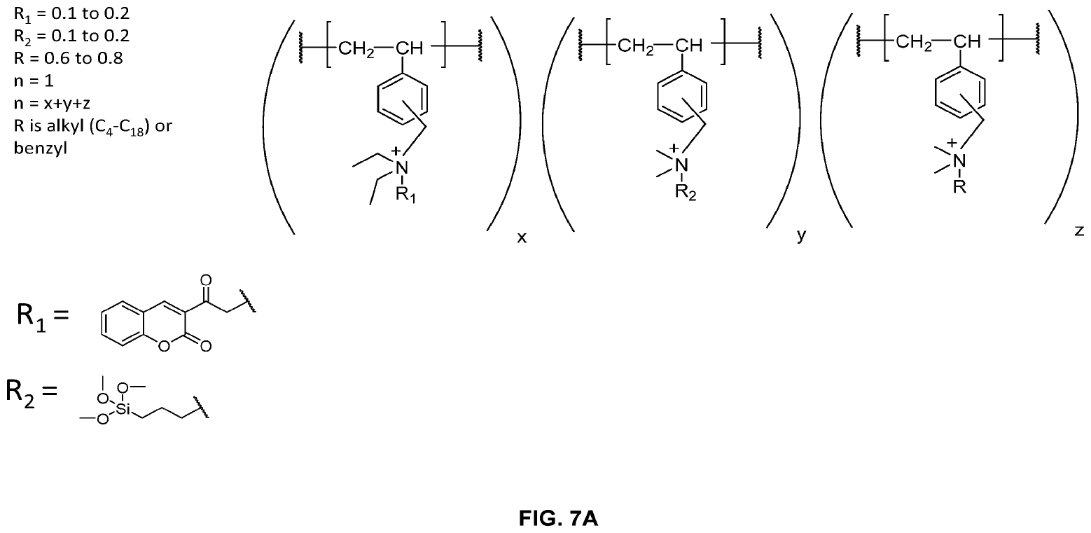

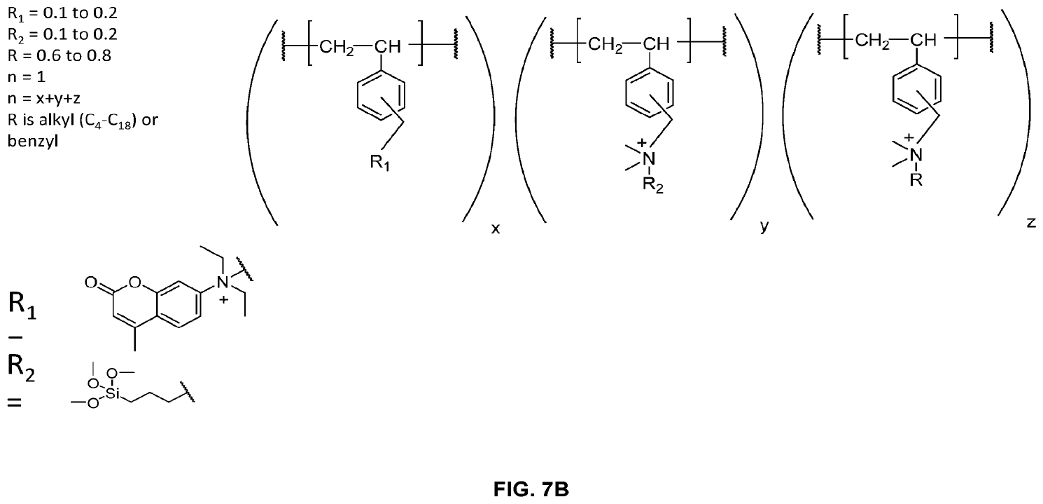

This Example describes a method to identify the presence of a grafted or coated layer by using a fluorescent marker covalently attached to a biocidal polymer comprising polyvinylbenzylchloride (PVBC) (FIGS. 7A-7B). The molar ratio of each moiety x, y, and z is as follows:

$$n = x + y + z = 1$$

$$x = 0.1 - 0.2 ;$$

$$y = 0.1 - 0.2 ; and$$

$$z = 0.6 - 0.8 .$$

### Experimental Protocol:

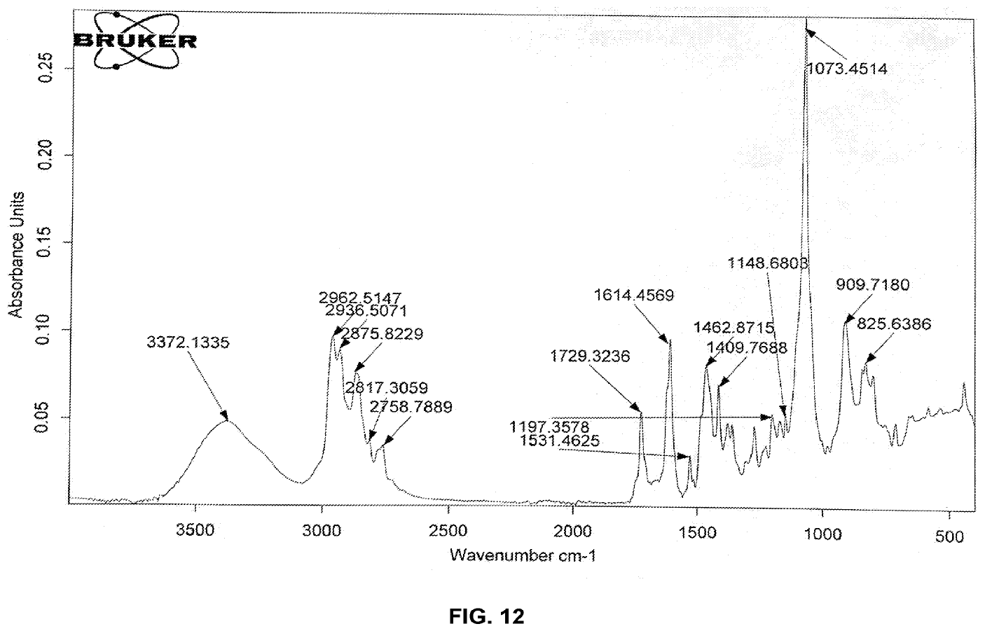

In a non-limiting example, the starting material, poly-(vinylbenzylchloride) (PVBC) (1 g, Mn=55,000, Ip=1.82) was dissolved in dry tetrahydrofuran (50 mL). Then N,N-dimethylaminopropyltrimethoxysilane (287 μL, 1.31 mmol) and 7-diethylamino-4-methylcoumarin (300 mg, 1.29 mmoles) were added in a 1% stoichiometric ratio, and the mixture was stirred under reflux during 6 hours at 50° C. N,N-dimethylbutylamine (3.30 mL, 23.6 mmol) in 30 mL of ethanol was added to the reaction solution. The reaction was allowed to stir for 12 h at 50° C. After the reaction was completed, THE was removed under reduced pressure to 5 mL and 100 mL of ethanol was added to the previous solution. FTIR-ATR (diamond): 2962 ν CH, 2931 ν CH, 2876 ν CH, 1737 C═O (coumarin), 1607 C═C (aromatic), 1473 νC—N+, 1071 (Si—O-Me) cm−1. FIG. 12 shows an image of the IR spectrum.

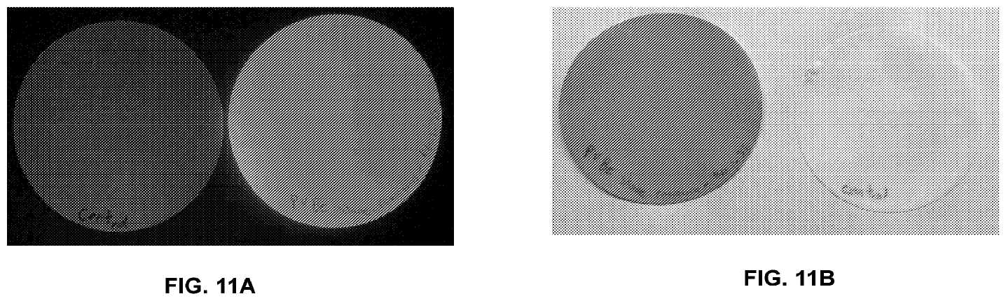

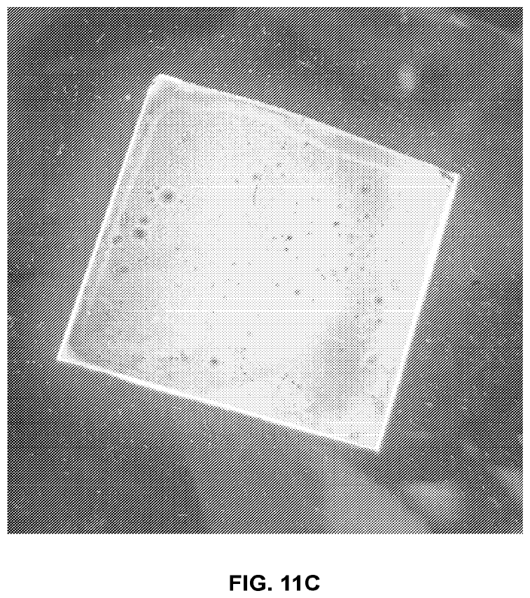

FIG. 11A illustrates an image comparing filter paper samples under UV light (365 nm): control (left) and treated (right) (treated with biocidal fluorescent quaternized polyvinylbenzylchloride of the disclosure). FIG. 11B illustrates an image comparing filter paper samples following a fluorescein test: control (right) and treated (left) (treated with biocidal fluorescent quaternized polyvinylbenzylchloride of the disclosure). FIG. 11C illustrates an image of a glass slide grafted with the biocidal fluorescent quaternized polyvinylbenzylchloride.

In a non-limiting example, the random copolymer was synthesized by quaternization of PVBC (Mw=55,000) first with 7-diethylamino-4-methylcoumarin then with a N,N dimethylaminopropyltrimethoxysilane (halogen: Br, I, or Cl). In a non-limiting example, 7-diethylamino-4-methylcoumarin is used since it is a commercially available compound (Sigma Aldrich). In some embodiments, the advantage of using 7-diethylamino-4-methylcoumarin, besides its availability, is its low cost in the current market when compared to other fluorescent dyes which are reactive to UV light. In a non-limiting example, any other fluorescent dyes could be used as needed for specific purposes based on the same chemical scheme, as would be understood by one of ordinary skill in the art.

In a non-limiting example, PVBC (1 Eq), N,N dimethylaminopropyltrimethoxysilane (0.1 Eq (A)), and 7-diethylamino-4-methylcoumarin 0.1 Eq (B) are added to THE and the reaction mixture is stirred for 6 hours at 50° C. Dimethylaminoalkane (R═C₄-C₁₂) in ethanol is then added to avoid the precipitation of the polycationic polymer, and then the reaction mixture is heated to reflux and stirred at reflux for 12 hours. The excess THE is then evaporated to provide an ethanolic solution of polycationic polymer product.

Such grafted or coated polymer may be visible to the naked eye by irradiating the layer with UV light (365 nm) which reflects blue fluorescent light.

### Example 6: Use of a Fluorescent Dipodal Silane as a Guest in a Biocidal Polymer Solution

This Example describes a method for identifying the presence of an aminated polymer-based grafted or coated layer by introducing a UV-active detectable moiety (guest molecule) into the prepared biocidal polymer solution used to prepare the layer. During curing, a covalent bond will form between the guest molecule and the biocidal polymer, thereby providing cross-linking. In non-limiting embodiments, this method can be used with coatings as well as surface grafting or modification.

### Experimental Protocol:

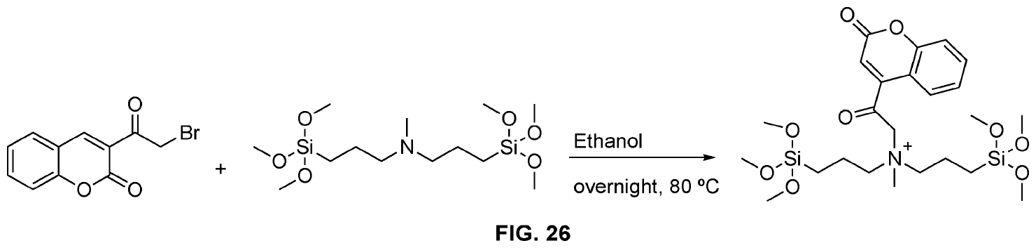

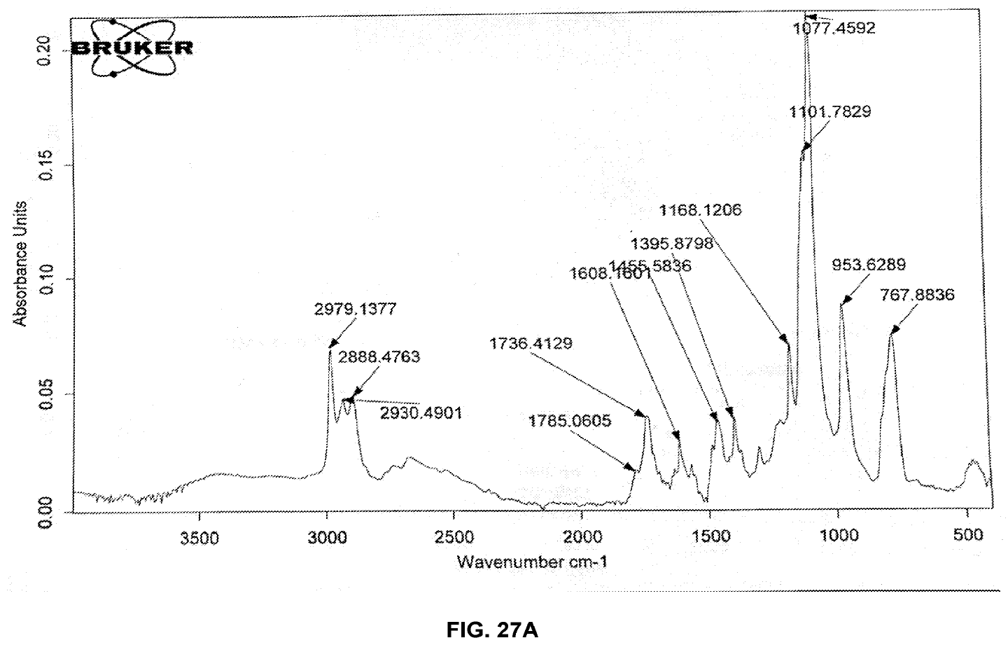

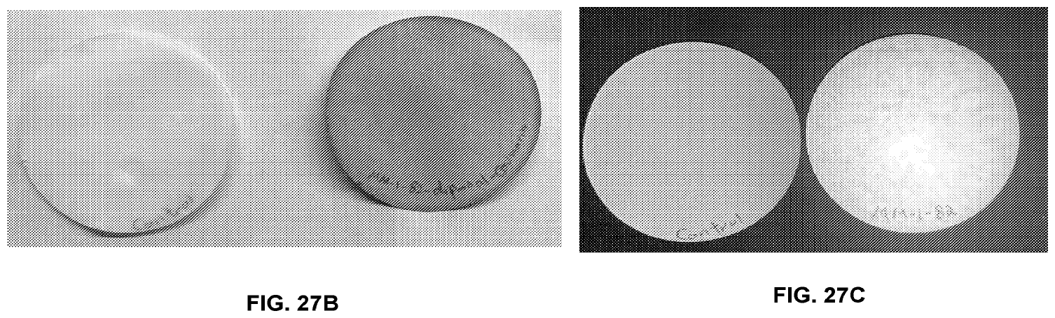

The fluorescent guest molecule was obtained by quaternization of 3-(bromoacetyl) coumarin and bis(3-trimethoxysilylpropyl-N-methylamine (FIG. 26). 3-(bromoacetyl) coumarin (275 mg, 1 mmol), and bis(3-trimethoxysilylpropyl)-N-methylamine (348 μL, 1 mmol) were dissolved in 30 mL of ethanol and the mixture was stirred under reflux overnight at 80° C. FTIR-ATR (diamond): 2979 CH₂, 2930 CH₂, 2888 CH₂, 1785 C═O (coumarin), 1736 C═O (coumarin), 1608 C═C (coumarin), 1455 ν C—N⁺, 1077 (Si—O-Me) cm⁻¹. FIG. 27A shows an image of the IR spectrum.

The resultant quaternary ammonium compound was obtained with a high yield.

A filter paper was impregnated with the fluorescent silane solution and dried at 100° C. for 10 minutes. A fluorescein test was performed on both a control filter paper and the treated filter paper sample (FIG. 27B). The control filter paper was prepared by impregnating filter paper with ethanol and drying for 10 minutes at 100° C. In contrast to the control which appeared white, the filter paper treated with the fluorescent silane solution appeared orange, demonstrating that the fluorescent silane was effectively grafted onto the filter paper.

Treated filter was observed under UV light (365 nm) and visually compared with a control. The treated filter exhibited markedly green fluorescence while the control did not shine under UV light. A comparison of filter paper samples under UV light is provided (FIG. 27C).

Following verification of the grafting ability of the fluorescent silane, a solution comprising a mixture of PVP-silane-coalkyl-PVP (made according to the method described in U.S. Pat. No. 10,238,110, which is incorporated by reference herein in its entirety) and the fluorescent dipodal silane in methanol in the ratio 1:1 w/w was prepared.

An activated glass slide is immersed in the prepared polymer solution (10 mg/mL). The solution is then evaporated to dryness in an oven at 120° C. The unbound copolymer was then removed by extensive washing with methanol and air-dried. The glass slide is observed under UV light (365 nm) and visually compared with a control.

### Example 7: Use of Fluorescent Silane as a Guest in a Biocidal Polymer Solution

This Example describes a method for identifying the presence of an aminated polymer-based grafted or coated layer by introducing a UV-active guest molecule into the prepared biocidal polymer solution used to prepare the layer. During curing, a covalent bond will form between the guest molecule and the biocidal polymer (cross-linking). This method can be used with coatings as well as surface grafting or modification.

### Experimental Protocol:

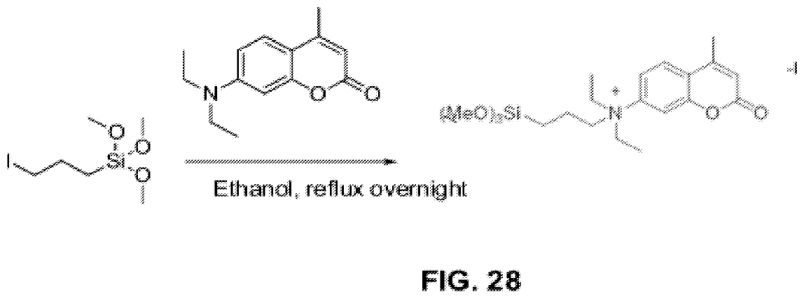

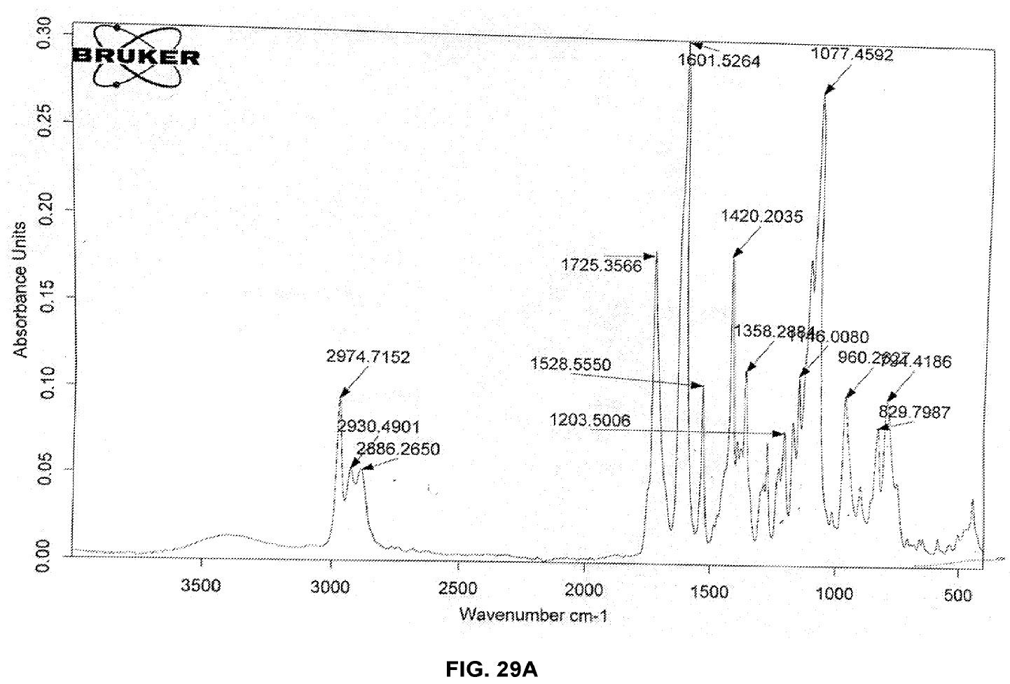

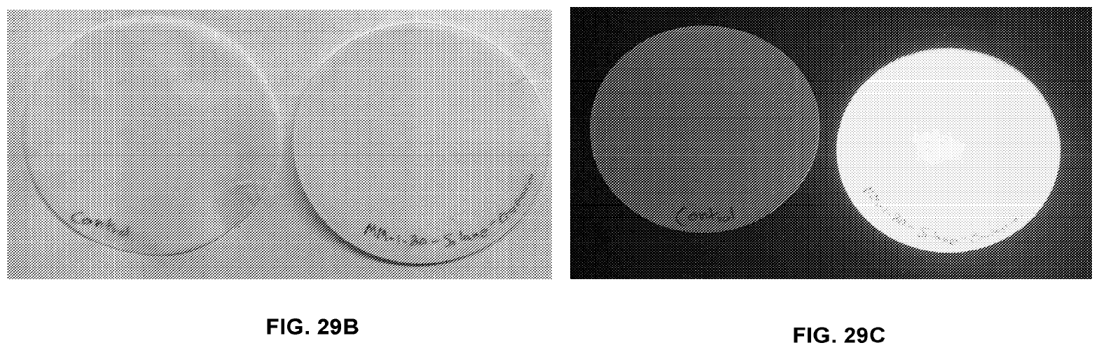

The fluorescent guest molecule was obtained by quaternization of 3-iodopropyl trimethoxysilane with 7-diethylamino-4-methylcoumarin (FIG. 28). 3-Iodopropyl trimethoxysilane (196 μL, 1 mmol) and 7-diethylamino-4-methylcoumarin (300 mg, 1.29 mmoles) were dissolved in 30 mL of ethanol and the mixture was stirred under reflux overnight at 80° C. FTIR-ATR (diamond): 2975 CH₂, 2930 CH₂, 2886 CH₂, 1725 C═O (coumarin), 1601C═C (coumarin), 1420 νC—N⁺, 1077 (Si—O-Me) cm−1. FIG. 29A shows an image of the IR spectrum.

The resultant quaternary ammonium compound was obtained with a high yield.

A filter paper was impregnated with the fluorescent silane solution and dried at 100° C. for 10 minutes. A fluorescein test was performed on both a control filter paper and the treated filter paper sample (FIG. 29B). The control filter paper was prepared by impregnating filter paper with ethanol and drying for 10 minutes at 100° C. In contrast to the control which appeared white, the filter paper treated with the fluorescent silane solution appeared yellow, demonstrating that the fluorescent silane was effectively grafted onto the filter paper. As shown in FIG. 29C, the filter paper treated with the fluorescent silane solution exhibited markedly blue fluorescence under UV light compared with a control.

Following verification of the grafting ability of the fluorescent silane, a solution comprising a mixture of PVP-silane-coalkyl-PVP (made according to the method described in U.S. Pat. No. 10,238,110, which is incorporated by reference herein in its entirety) and the fluorescent silane in methanol in the ratio 1:1 w/w was prepared.

An activated glass slide is immersed in the prepared polymer solution (10 mg/mL). The solution is then evaporated to dryness in an oven at 120° C. The unbound copolymer is then removed by extensive washing with methanol and air-dried. The glass slide is observed under UV light (365 nm) and visually compared with a control. The slide coated with the prepared polymer solution exhibits fluorescence under UV light.

### Example 8: Use of Fluorescent Dipodal Silane as a Guest in a Biocidal Polymer Solution

This Example describes a method for identifying the presence of an aminated polymer-based grafted or coated layer by introducing a UV-active guest molecule into the prepared biocidal polymer solution used to prepare the layer. During curing, a covalent bond will form between the guest molecule and the biocidal polymer (cross-linking). In a non-limiting example, this method can be used with coatings as well as surface grafting or modification.

### Experimental Protocol:

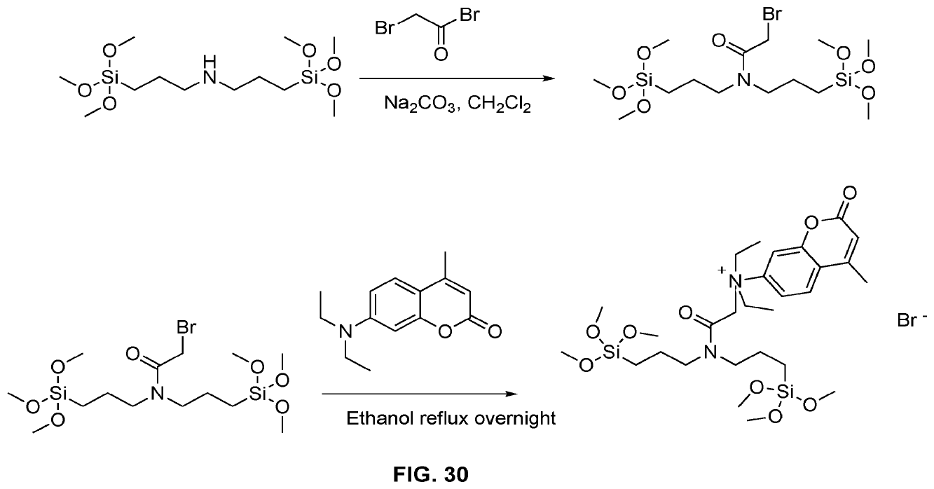

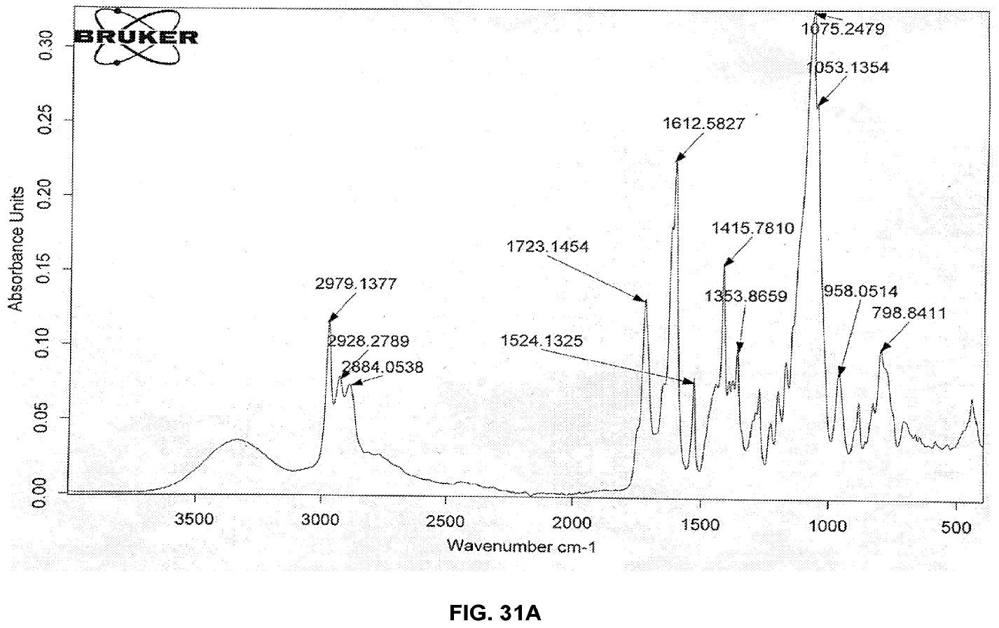

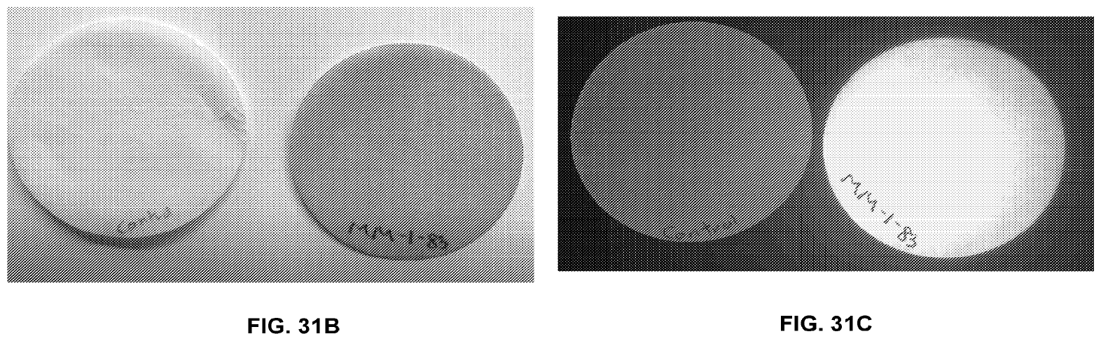

The fluorescent guest molecule was obtained by quaternization of N, N-Bis[3-(trimethoxysilylpropyl) bromoacetamide with 7-diethylamino-4-methylcoumarin (FIG. 30). A solution of Bromoacetylbromide (2.5 mL, 20 mmol) in 10 mL of dry dichloromethane was added slowly to a Bis[3-(trimethoxysilylpropyl amine (6.45 g, 20 mmol) in dichloromethane (20 mL) at room temperature. The reaction was allowed to stir overnight. After the reaction completion, dichloromethane was removed under reduced pressure and 50 mL of ethanol was added to the residue. 800 mg of potassium hydroxide in 5 mL of ethanol was added slowly to solution and formed a precipitate. The precipitate was filtered and the filtrate was taken into the next step directly. FTIR-ATR (diamond): 2981 CH₂, 2941 CH₂, 2888 CH₂, 2786 CH₃, 1743 C═0, 1015 (Si—O-Me) cm⁻¹. 7-diethylamino-4-methylcoumarin (300 mg, 1.29 mmoles) was added to the solution of N, N-Bis[3-(trimethoxysilylpropyl) bromoacetamide (462 mg, 1 mmol) in 35 mL of ethanol. The reaction mixture was allowed to stir over night at 80° C. FTIR-ATR (diamond): 2979 CH₂, 2928 CH₂, 2884 CH₂, 1723 C═O (coumarin), 1415 νC—N⁺, 1015 (Si—O-Me) cm⁻¹. FIG. 31A shows an image of the IR spectrum.

The resultant quaternary ammonium compound was obtained with a high yield.

A filter paper was impregnated with the fluorescent silane solution and dried at 100° C. for 10 minutes. A fluorescein test was performed on both a control filter paper and the treated filter paper sample (FIG. 31B). The control filter paper was prepared by impregnating filter paper with ethanol and drying for 10 minutes at 100° C. In contrast to the control which appeared white, the filter paper treated with the fluorescent silane solution appeared orange, demonstrating that the fluorescent silane was effectively grafted onto the filter paper.

Following verification of the grafting ability of the fluorescent silane, a solution comprising a mixture of PVP-silane-coalkyl-PVP (made according to the method described in U.S. Pat. No. 10,238,110, which is incorporated by reference herein in its entirety) and the fluorescent silane in methanol in the ratio 1:1 w/w was prepared.

### Example 9: One-Pot Synthesis and Deposition of Graftable Fluorescent Biocidal Non-Aminated Polymers (Catechol-Based)

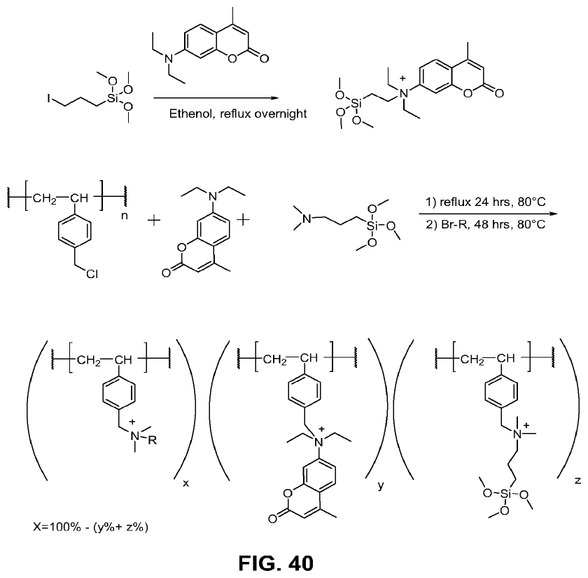

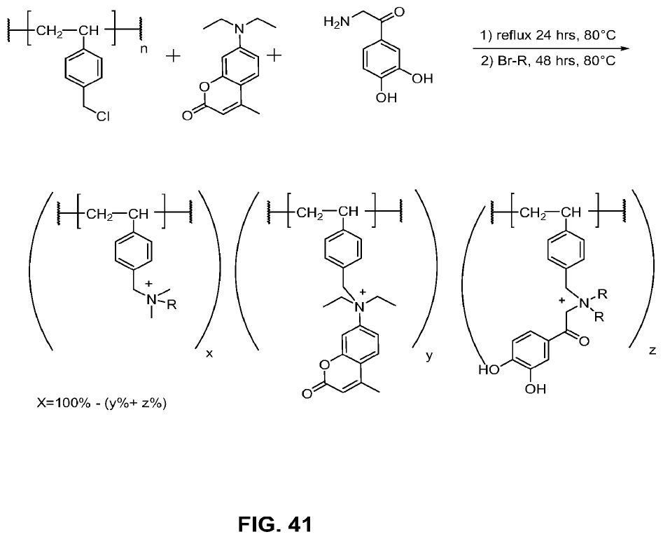

This Example describes exemplary methods to identify the presence of a grafted or coated layer by using a fluorescent marker covalently attached to a biocidal polymer comprising polyvinylbenzylchloride (PVBC) (FIGS. 39-40). In some embodiments, the molar ratio of each moiety x, y, and z is as follows:

$$n = x + y + z = 1$$

$$x = 0.1 - 0.2 ;$$

$$y = 0.1 - 0.2 ; and$$

$$z = 0.6 - 0.8 .$$

### Example 10: Dye-Labelled Graftable PEI-Based Biocidal Polymer (Silane-Based)

This Example describes exemplary method for identifying the presence of an aminated polymer-based grafted or coated layer by covalently attaching a fluorescent marker to a biocidal polymer using polyethylenimine (PEI).

The random copolymer is synthesized by quaternization of methyl-polyethylenimine (PEI) first with halogenoacetylcoumarin then with a halogenopropyltrimethoxysilane (halogen: Br, I, or Cl). In a non-limiting example, 3-bromoacetylcoumarin is used since it is a commercially available compound (Sigma-Aldrich catalogue). In some embodiments, the advantage of using 3-bromoacetylcoumarin, besides its availability, is its low cost in the current market when compared to other fluorescent dyes which are reactive to UV light. Any other fluorescent dyes could be used as needed for specific purposes based on the chemical scheme, as would be understood by one of ordinary skill in the art.

The quaternization is performed in refluxing ethanol/methanol for 3 to 6 hours with a mixture of halogenoacetylcoumarin and a halogenotrialkoxysilane. Then, a bromoalkyl chain or benzylbromide is added to complete the quaternization for another period (24 h to 96 h).

Such grafted or coated polymer may be visible to the naked eye by shining the layer with UV light (365 nm) which reflects yellow fluorescent light.

### Experimental Protocol:

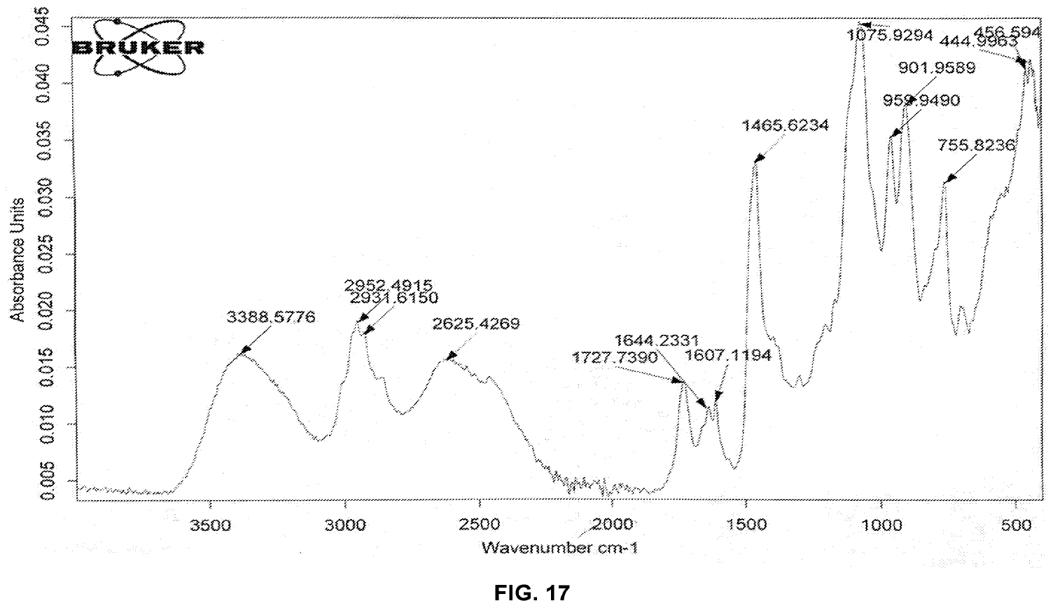

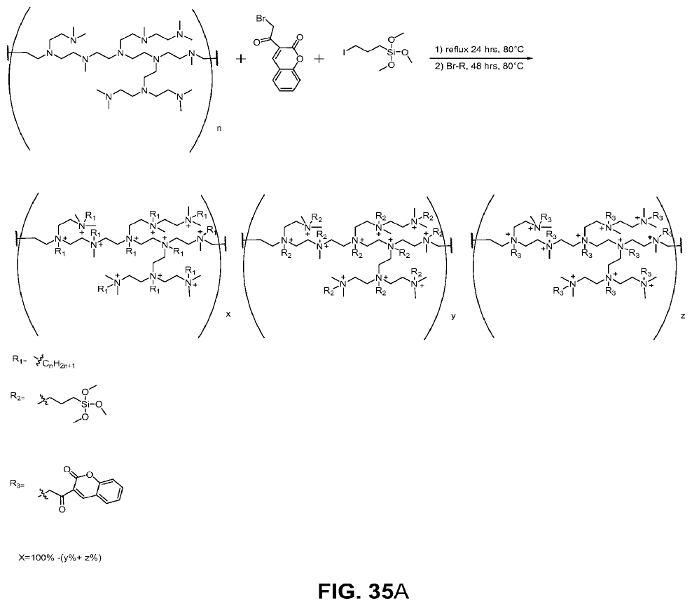

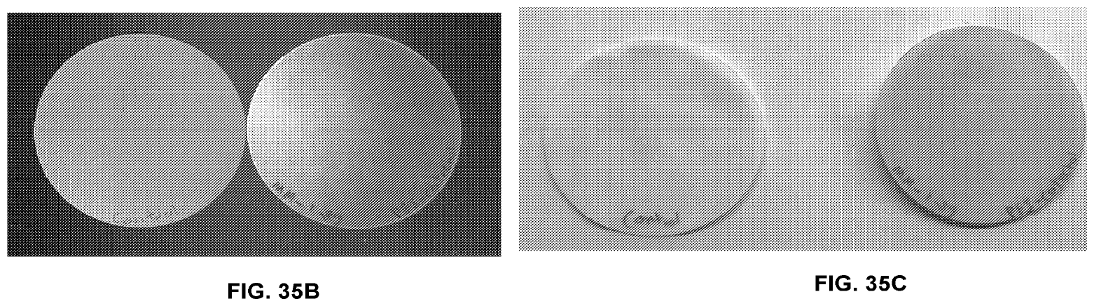

Methyl PEI (0.5 g, Mn=75,000) in 30 mL of ethanol was added to a round bottom flask, and then 3-(bromoacetyl)coumarin (200 mg, 0.75 mmol) and 3-iodopropyl trimethoxysilane (250 μL, 1.28 mmol) were added in 10% stoichiometric ratio. The mixture was then stirred under reflux overnight at 80° C. 1-bromohexane (2 mL, 8 mmol) in 10 mL of ethanol was added to the reaction solution. The reaction was allowed to stir for two days at 80° C. FTIR-ATR (diamond): 2952 ν CH₂, 2931 ν CH₂, 2625 ν CH₂, 1727 ν C═O (coumarin), 1644 ν C═O (coumarin), 1607 ν C═C (coumarin or catechol), 1465 ν C—N+, 1075 (Si—O-Me) cm⁻¹. FIG. 17 shows an image of the IR spectrum. An exemplary reaction scheme is shown in FIG. 35.

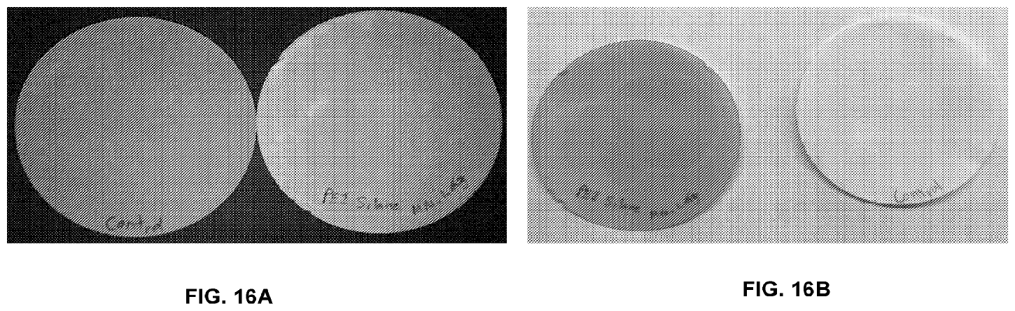

A filter paper was impregnated with the fluorescent catechol solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange. Treated filter was observed under UV light (365 nm) and visually compared with a control. The treated filter exhibited markedly blue fluorescence while the control did not shine under UV light. FIGS. 16A and 16B show images of a comparison of filter paper samples under UV light (FIG. 16A) and fluorescein test (FIG. 16B).

In some embodiments, 3-(bromoacetyl) coumarin can be replaced with 2-oxo-2H-chromen-7-yl 2-bromoacetate:

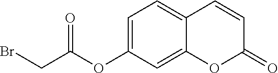 [SMILES: O=C(CBr)Oc1ccc2ccc(=O)oc2c1]

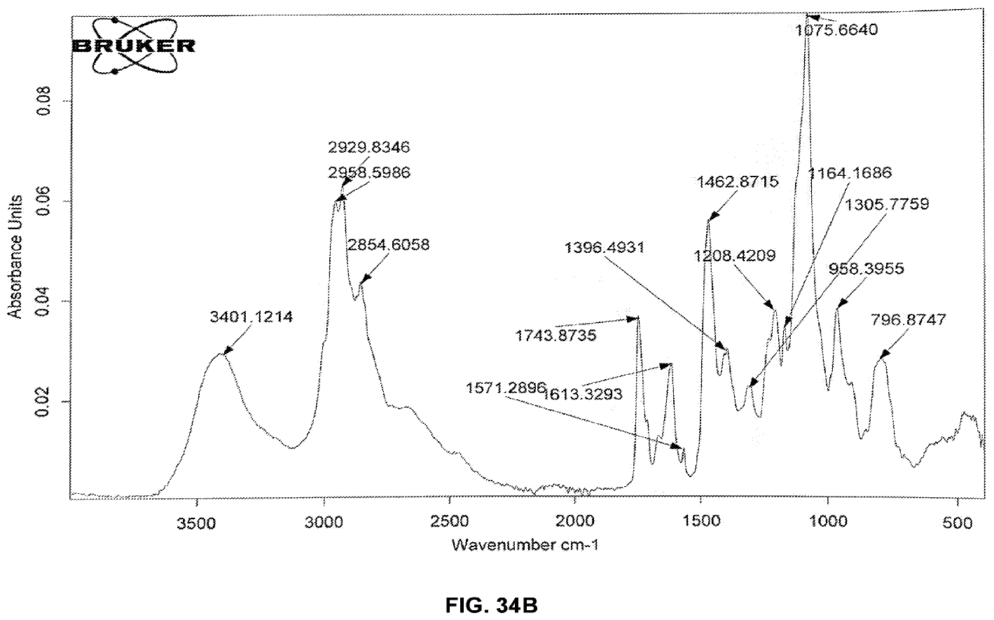

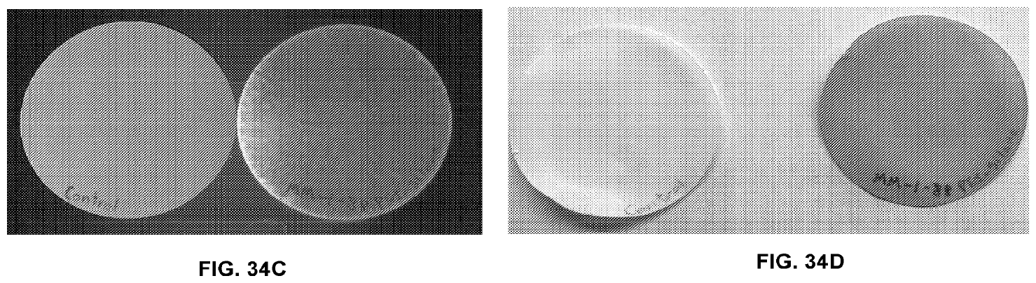

(see FIG. 34A as an non-limiting example).

Experimental protocol: Methyl PEI (0.5 g, Mn=75,000) in 30 mL of ethanol was added to a round bottom flask, and then 2-oxo-2H-chromen-7-yl 2-bromoacetate (211 mg, 0.75 mmol) and 3-iodopropyl trimethoxysilane (260 μL, 1.28 mmol) were added in 10% stoichiometric ratio. The mixture was then stirred under reflux overnight at 80° C. 1-bromohexane (2 mL, 8 mmol) in 10 mL of ethanol was added to the reaction solution. The reaction was allowed to stir for one day at 80° C. FTIR-ATR (diamond): 2958 ν CH₂, 2929 ν CH₂, 2854 ν CH₂, 1743 ν C═O (coumarin), 1607 ν C═C, 1465 ν C—N+, 1075 (Si—O-Me) cm⁻¹. FIG. 34B shows the IR spectrum.

A filter paper was impregnated with the fluorescent catechol solution and dried at 100° C. for 10 minutes. A fluorescein test was performed on both a control filter paper and the treated filter paper sample. Treated filter was observed under UV light (365 nm) and visually compared with a control. The treated filter exhibited fluorescence while the control did not shine under UV light. FIGS. 34C and 34D show images of a comparison of filter paper samples under UV light (FIG. 34C) and fluorescein test (FIG. 34D).

### Example 11: Dye-Labelled Graftable PEI-Based Biocidal Polymer (Catechol-Based)

This Example describes a method for identifying the presence of a grafted or coated layer by covalently attaching a fluorescent marker to a biocidal polymer using polyethylenimine (PEI).

The random copolymer is synthesized by quaternization of methyl-PEI first with halogenoacetylcoumarin then with a halogenoacetylcatechol (halogen: Br, I, or Cl). In a non-limiting example, 3-bromoacetylcoumarin is used since it is a commercially available compound (Sigma-Aldrich catalogue). In some embodiments, the advantage of using 3-bromoacetylcoumarin, besides its availability, is its low cost in the current market when compared to other fluorescent dyes which are reactive to UV light. Any other fluorescent dyes could be used as needed for specific purposes based on the same chemical scheme, as would be understood by one of ordinary skill in the art.

The quaternization is performed in refluxing ethanol/methanol for 3 to 6 hours with a mixture of halogenoacetylcoumarin and a halogenoacetylcatechol. Then, a bromoalkyl chain or benzylbromide is added to complete the quaternization for another period (24 h to 96 h).

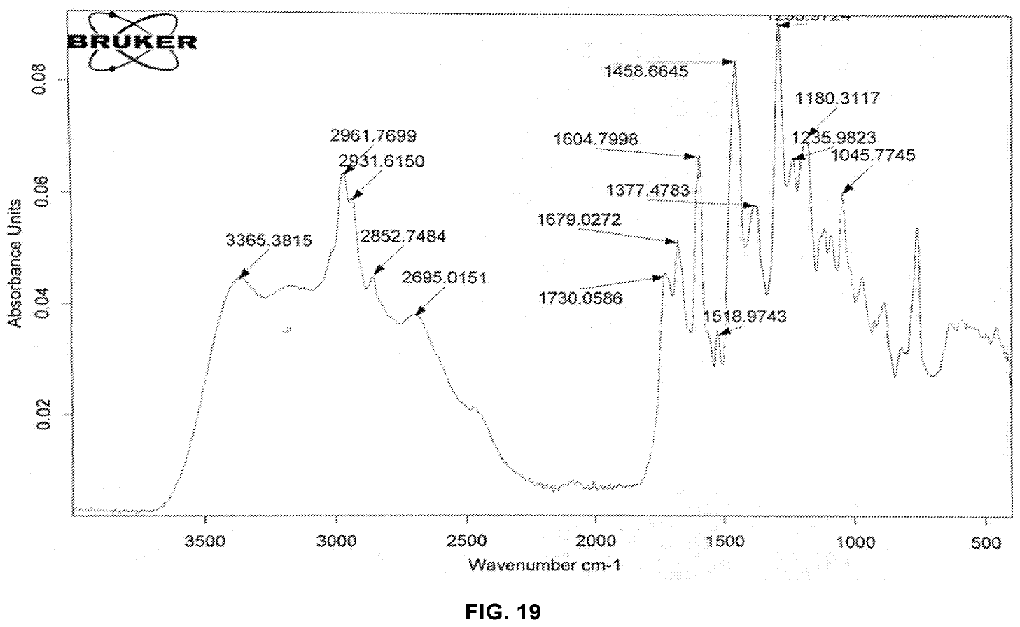

The grafted or coated polymer may be visible to the naked eye by irradiating the layer with UV light (365 nm) which reflects yellow fluorescent light. FIG. 19 shows an image of the IR spectrum.

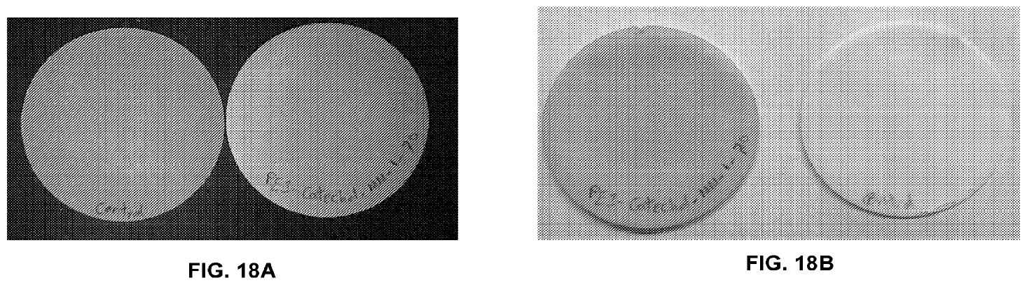

A filter paper was impregnated with the fluorescent catechol-based polymer solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange. Treated filter was observed under UV light (365 nm) and visually compared with a control. The treated filter exhibited markedly yellow fluorescence while the control did not shine under UV light. FIGS. 18A and 18B show images of a comparison of filter paper samples under UV light (FIG. 18A) and fluorescein test (FIG. 18B).

### Experimental Protocol:

Methyl PEI (641 mg, Mn=75,000) in 73 mL of ethanol was added to a round bottom flask, and then 3-(bromoacetyl) coumarin (534 mg, 2 mmol) and 2-chloro 3,4-dihydroxyacetophenone (373 mg, 2 mmol) were added in 10% stoichiometric ratio, and the mixture was stirred under reflux overnight at 80° C. 1-bromohexane (2 mL, 8 mmol) in 10 mL of ethanol was added to the reaction solution. The reaction was allowed to stir for two days at 80° C. FTIR-ATR (diamond): 3365 ν OH (catechol), 2961 νCH₂, 2931 νCH₂, 2853 νCH₂, 2695 νCH₂, 1730 ν C═O (coumarin), 1679 ν C═O (coumarin), ν 1644 C═O (catechol), 1604 ν C═C (coumarin or coumarin), 1465 νC—N+, 1298 ν C═C (catechol) cm⁻¹. FIG. 19 shows the IR spectrum. An exemplary reaction scheme is shown in FIG. 33.

A filter paper was impregnated with the resulting fluorescent catechol-based polymer solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange. A filter paper was impregnated with the fluorescent catechol solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange (FIG. 18B).

Treated filter was observed under UV light (365 nm) and visually compared with a control. The treated filter exhibited markedly green fluorescence while the control did not shine under UV light (FIG. 18A).

In some embodiments, 3-(bromoacetyl) coumarin can be replaced with 2-oxo-2H-chromen-7-yl 2-bromoacetate:

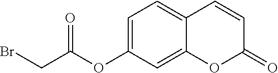 [SMILES: O=C(CBr)Oc1ccc2ccc(=O)oc2c1]

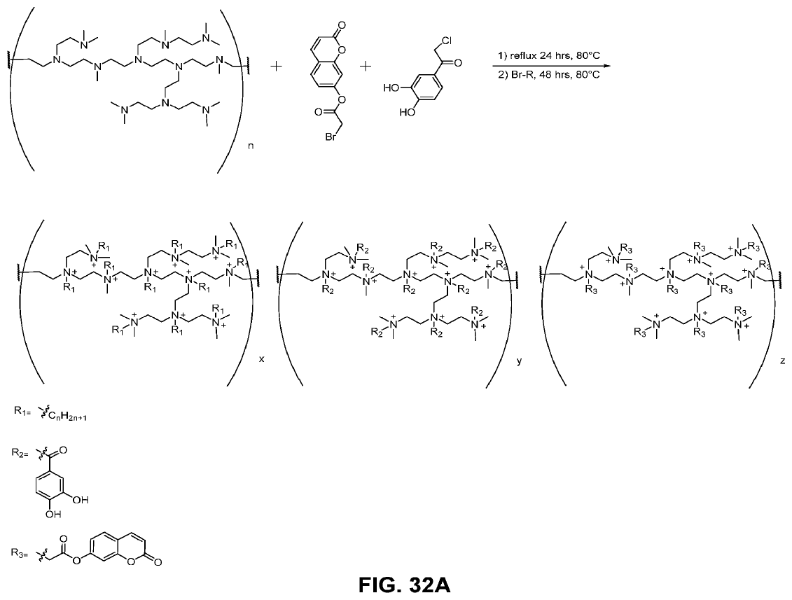

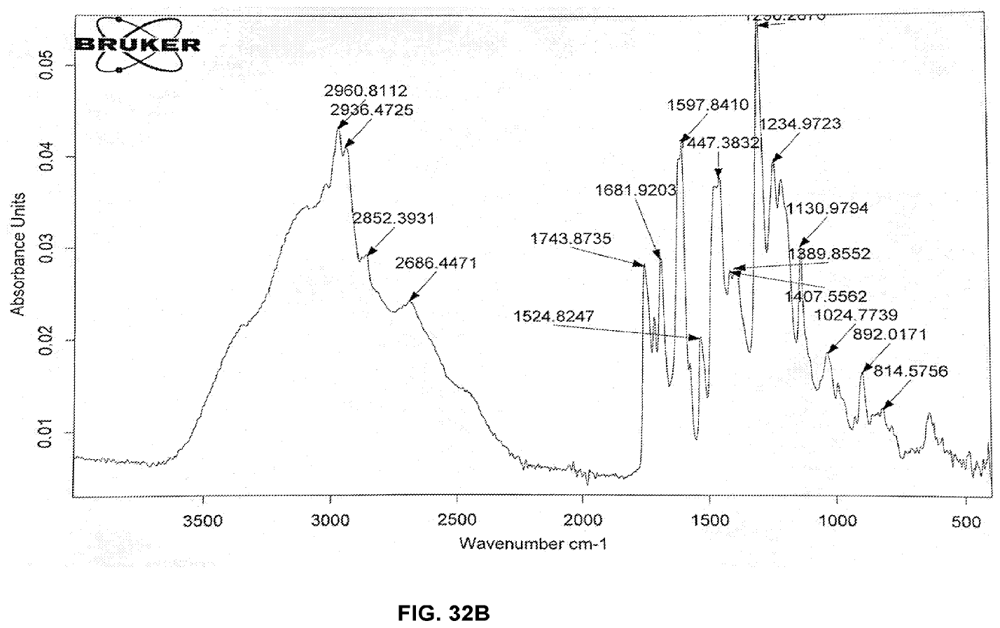

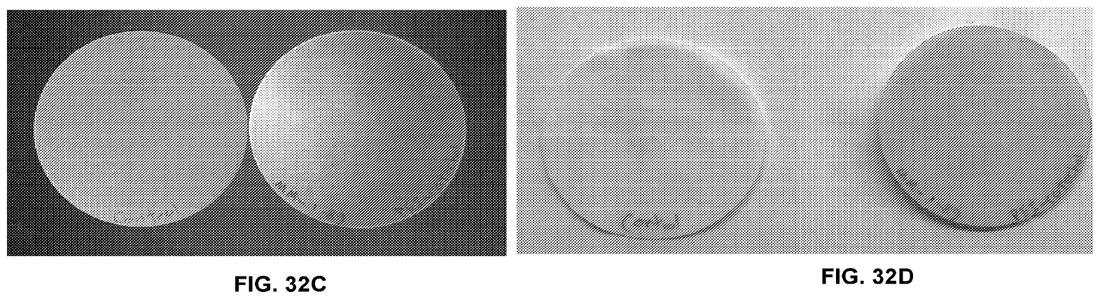

(see FIG. 32A as an non-limiting example).

Experimental protocol: Methyl PEI (0.5 g, Mn=75,000) in 30 mL of ethanol is added to a round bottom flask, and then 2-oxo-2H-chromen-7-yl 2-bromoacetate (211 mg, 0.75 mmol) and 2-chloro 3,4-dihydroxyacetophenone (247 mg, 1.32 mmol) are added in 10% stoichiometric ratio. The mixture is then stirred under reflux overnight at 80° C. 1-bromohexane (2 mL, 8 mmol) in 10 mL of ethanol is added to the reaction solution. The reaction is allowed to stir for one day at 80° C. FTIR-ATR (diamond): FIG. 32B shows the IR spectrum.

A filter paper was impregnated with the fluorescent catechol solution and dried at 100° C. for 10 minutes. A fluorescein test was performed on both a control filter paper and the treated filter paper sample. Treated filter was observed under UV light (365 nm) and visually compared with a control. The treated filter exhibited markedly green fluorescence while the control did not shine under UV light. FIGS. 35B and 35D show images of a comparison of filter paper samples under UV light (FIG. 32D) and fluorescein test (FIG. 32C).

### Example 12: Dye-Labelled Graftable C18 Quaternary Ammonium Compounds (Silane-Based)

This Example describes a method for identifying the presence of a grafted or coated layer by covalently attaching a fluorescent marker to an antimicrobial C18 quaternary ammonium compound.

### Experimental Protocol:

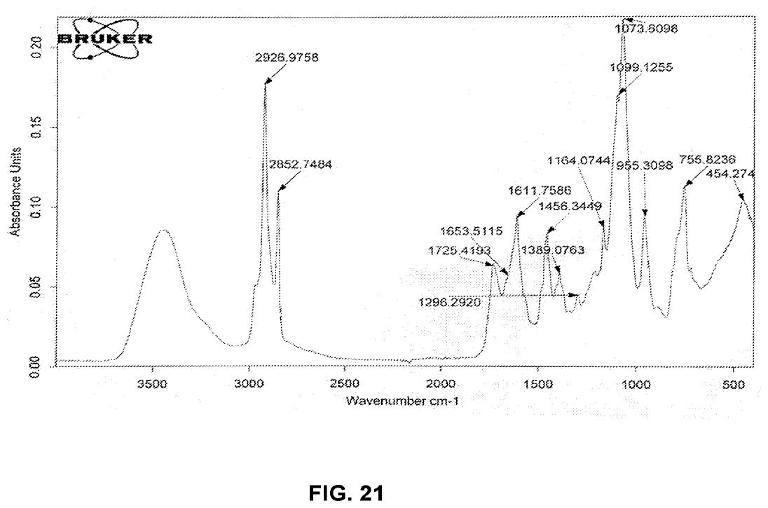

N-methyloctadecylamine (283 mg, 1 mmol), 3-(bromoacetyl) coumarin (275 mg, 1 mmol) and 3-iodopropyl trimethoxysilane (196 μL, 1 mmol) and Na₂CO₃ (330 mg, 3.11 mmol) were dissolved in 30 mL of ethanol and the mixture was stirred under reflux overnight at 80° C. FTIR-ATR (diamond): 2920 νCH₂, 2850 νCH₃, 1737 ν C═O (coumarin), 1653 ν C═O (coumarin), 1607 ν C═C (coumarin), 1463 νC—N⁺, 1076 (Si—O-Me) cm¹. FIG. 21 shows the IR spectrum.

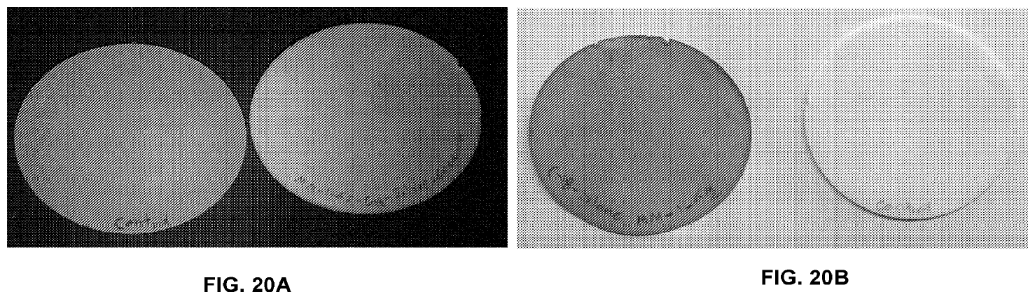

A filter paper was impregnated with the fluorescent silane-based C18 quaternary ammonium compound solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange (FIG. 20B).

Treated filter was observed under UV light (365 nm) and visually compared with a control. The treated filter exhibited markedly green fluorescence while the control did not shine under UV light (FIG. 20A).

### Example 13: Dye-Labelled Graftable C18 Quaternary Ammonium Compounds (Catechol-Based)

This Example describes a method for identifying the presence of a grafted or coated layer by covalently attaching a fluorescent marker to an antimicrobial C18 quaternary ammonium compound.

### Experimental Protocol:

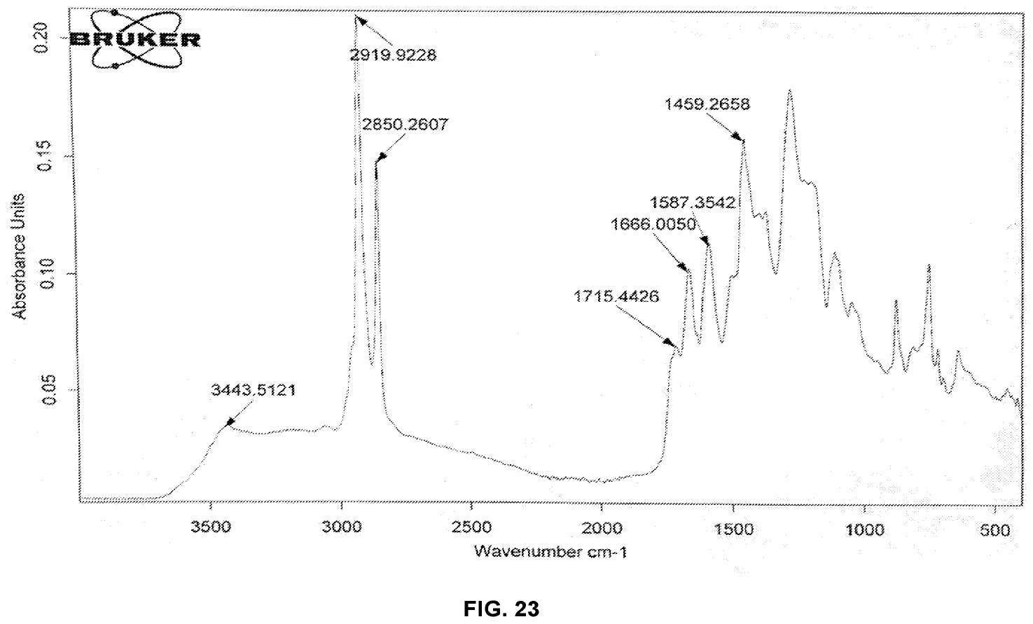

N-methyloctadecylamine (283 mg, 1 mmol), 3-(bromoacetyl) coumarin (275 mg, 1 mmol) 2-chloro 3,4-dihydroxyacetophenone (194 mg, 1 mmol) and Na₂CO₃ (330 mg, 3.11 mmol) were dissolved in 30 mL of ethanol and the mixture was stirred under reflux overnight at 80° C. FTIR-ATR (diamond): 3437 ν OH (catechol), 2920 νCH₂, 2850 νCH₃, 1716 ν C═O (coumarin), 1662 ν C═O (coumarin), 1590 ν C═C (coumarin or catechol), 1458 νC—N+, 1275 ν C═C (catechol) cm⁻¹. FIG. 23 shows the IR spectrum.

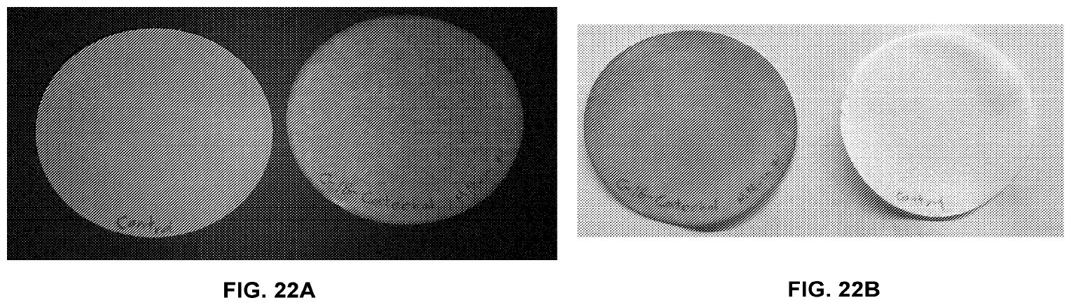

A filter paper was impregnated with the fluorescent catechol-based C18 quaternary ammonium compound solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange (FIG. 22B).

Treated filter was observed under UV light (365 nm) and visually compared with a control. The treated filter exhibited markedly green fluorescence while the control did not shine under UV light (FIG. 22A).

### Example 14: Dye-Labelled Graftable C10 Quaternary Ammonium Compounds (Dipodal Silane-Based)

This Example describes a method for identifying the presence of a grafted or coated layer by covalently attaching a fluorescent marker to a dipodal-silane based antimicrobial C10 quaternary ammonium compound.

### Experimental Protocol:

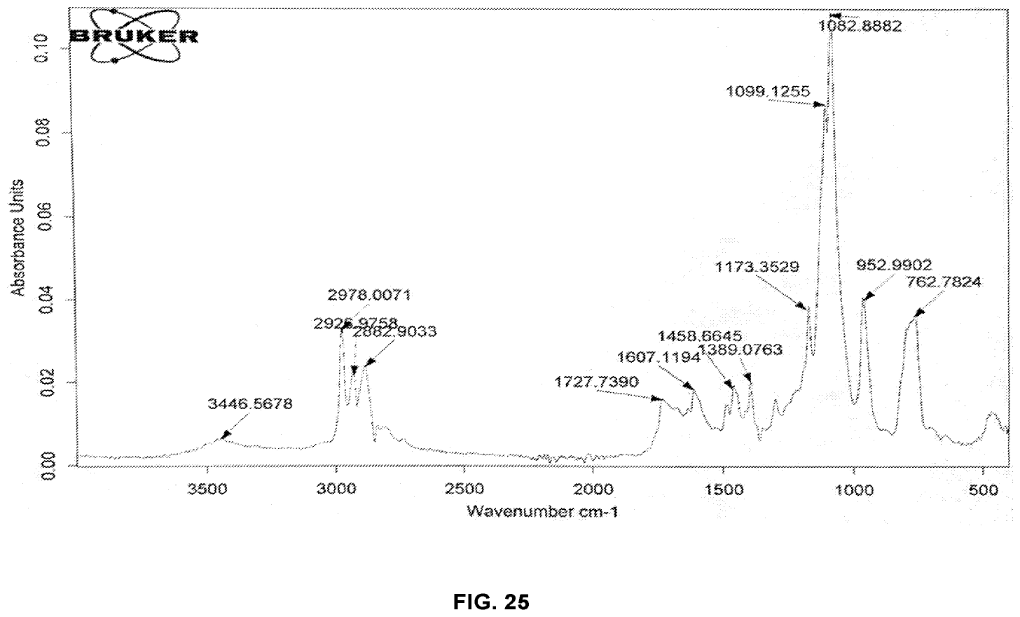

1-bromodecane (221 mg, 1 mmol), 3-(bromoacetyl) coumarin (275 mg, 1 mmol), Bis[3-(trimethoxysilyl)propyl]amine (328 μL, 1 mmol) and Na₂CO₃ (330 mg, 3.11 mmol) were dissolved in 30 mL of ethanol and the mixture was stirred under reflux overnight at 80° C. FTIR-ATR (diamond): 2978 CH₂, 2926 CH₂, 2882 CH₃, 1727 C═O (coumarin), 1607 C═C (coumarin), 1458 νC—N⁺, 1082 (Si—O-Me) cm⁻¹. FIG. 25 shows the IR spectrum.

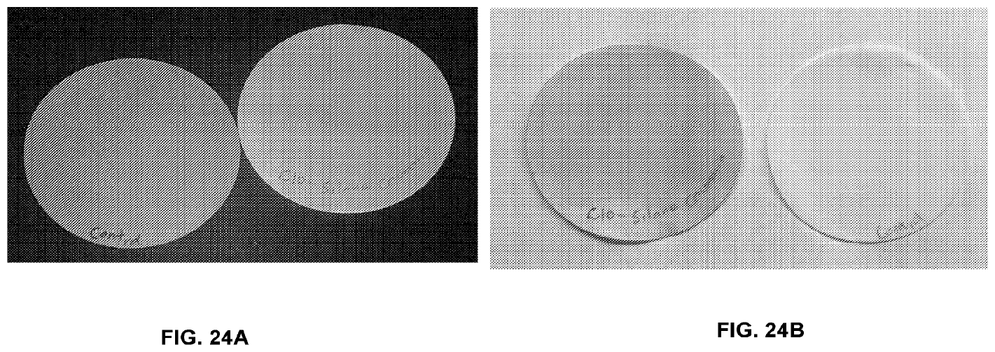

A filter paper was impregnated with the fluorescent dipodal silane-based C10 quaternary ammonium compound solution and dried at 100° C. for 10 minutes. A fluorescein test was performed on both a control filter paper and the treated filter paper sample. The control filter paper was prepared by impregnating filter paper with ethanol and drying for 10 minutes at 100° C. In contrast to the control filter paper, the fluorescent biocidal polymer was effectively grafted on filter paper which appeared orange (FIG. 24B).

Treated filter was observed under UV light (365 nm) and visually compared with a control. The treated filter exhibited markedly green fluorescence while the control did not shine under UV light (FIG. 24A).

### REFERENCES

1. Kügler R., Bouloussa O., Rondelez F. Evidence of a Charge-Density Threshold for Optimum Efficiency of Biocidal Cationic Surfaces. Microbiology 2005 May; 151(Pt 5):1341-1348.
2. Oh J. Y., Cho I. H., Lee H., Park K-J., Lee H., Park S. Y. 2012, Bio-inspired catechol chemistry: a new way to develop a rer-moldable and injectable coacervate hydrogel. Chem. Comm. 48:11895-11897.
3. Elena P., Miri K. 2018, Formation of contact active antimicrobial surfaces by covalent grafting of quaternary ammonium compounds. Colloids and Surfaces B: Biointerfaces 169:195-205.
4. Kim S., Nam J. A., Lee S., In I., Park, S. Y. 2014, Amtimicrobial activity of water resistant surface coating from catechol conjugated polyquaternary amine on versatile substrates. J. Appl. Polym. Sci. 131:40708.
5. Kim S. H., Lee S., In I., Park, S. Y. 2016, Synthesis and antibacterial activity of surfacecoated catechol-conjugated polymer with silver nanoparticles on versatile substrate. Surf. Interface Anal. 9:995-1001.
6. Le T.-N., Au-Duong A.-N., Lee C.-K. 2019, Facile coating on microporous polypropylene membrane for antifouling microfiltration using comb-shaped poly(N-vinaylpyrrolidone) with multivalent catechol. J. Membrane Science 574:164-173.
7. Wu Z., Wang J., Pei D., Li L., Mu Y., Wan X. 2018, A simple strategy to achieve mussel-inspired highly effective antibacterial coating. Macromol. Mater. Eng. 303:1700430.
8. Nam J. A., Nahain A.-A., Kim S. M., In I., Park S. Y. 2013, Successful stabilization of functionalized hybrid graphene for high-performance antimicrobial activity. Acta Biomaterialia 9:7996-8003.
9. Jeong C. J., In, I. Park S. Y. 2014, Facile preparation of metal nanoparticle-coated polystyrene beads by catechol conjugated polymer. Surf Interface Anal. 47:253-258.
10. Bouloussa et al., “A new, simple approach to confer permanent antimicrobial properties to hydroxylated surfaces by surface functionalization,” (2008) Chem. Commun. pp. 951-953.

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

1. A polymer comprising one or more detectable moieties.

2. The polymer of claim 1, wherein the detectable moiety is one or more selected from a fluorescent moiety, a phosphorescent moiety, and a luminescent moiety.

3. The polymer of claim 1, wherein the detectable moiety is a fluorescent moiety selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety.

4. The polymer of any of claims 1-3, wherein the detectable moiety is a moiety of formula (I), formula (II), or formula (VII): 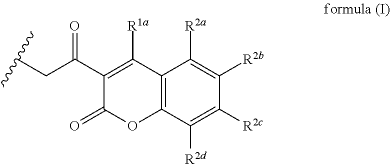
wherein in formula (I):
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; 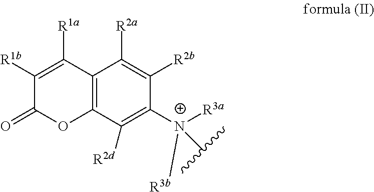
wherein in formula (II):
each R^{1a} and R^{1a} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; 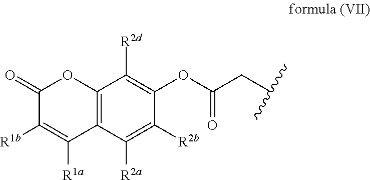
wherein in formula (VII):
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

5. The polymer of claim 4, wherein the detectable moiety is selected from formula (10), formula (20), formula (21), and formula (70): 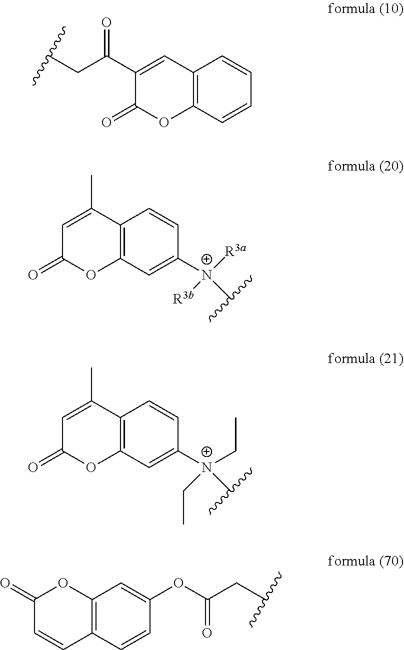
wherein in formula (20), R^{3a} and R^{3b} are each independently optionally substituted alkyl.

6. The polymer of claim 4 or claim 5, wherein R^{3a} and R^{3b} are each ethyl.

7. The polymer of claim 1, wherein the polymer comprises at least one moiety of formula (Iaa), at least one moiety of formula (3), and at least one moiety of formula (4): 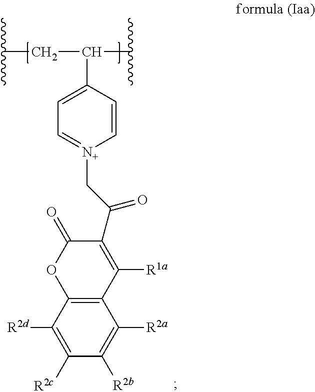
wherein in formula (Iaa):
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; 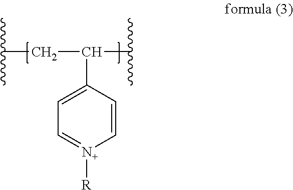
wherein in formula (3):
R is C₄-C₁₈ alkyl or benzyl; 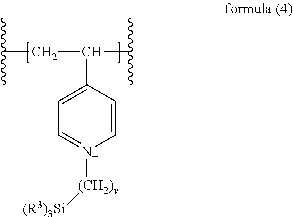
wherein in formula (4):
each R³ is independently optionally substituted alkoxy; and
v is an integer from 3 to 10.

8. The polymer of claim 1 or 7, wherein the polymer comprises at least one moiety of formula (Ia), at least one moiety of formula (3), and at least one moiety of formula (4): 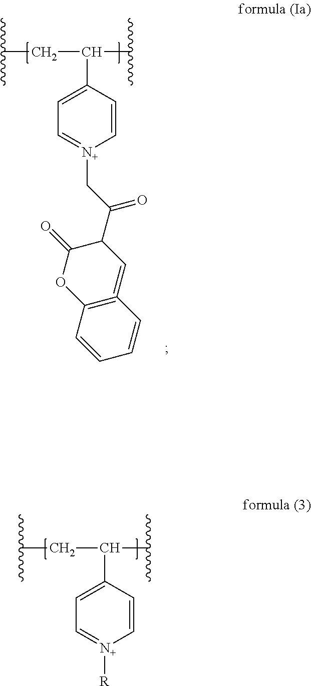
wherein in formula (3):
R is C₄-C₁₈ alkyl or benzyl; 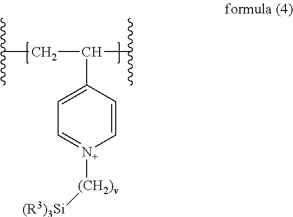
wherein in formula (4):
each R³ is independently optionally substituted alkoxy; and
v is an integer from 3 to 10.

9. The polymer of claim 1, wherein the polymer comprises at least one moiety of formula (VIIa), at least one moiety of formula (3), and at least one moiety of formula (4): 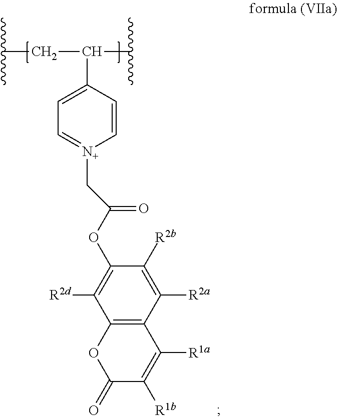
wherein in formula (VIIa):
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; 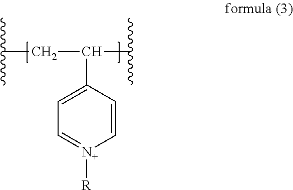
wherein in formula (3):
R is C₄-C₁₈ alkyl or benzyl; 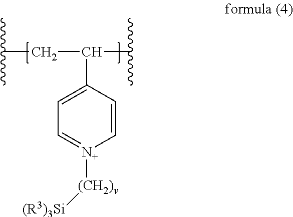
wherein in formula (4):
each R³ is independently optionally substituted alkoxy; and
v is an integer from 3 to 10.

10. The polymer of claim 1 or 9, wherein the polymer comprises at least one moiety of formula (VIIb), at least one moiety of formula (3), and at least one moiety of formula (4): 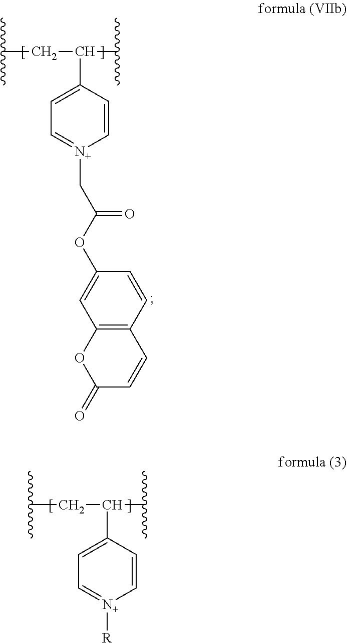
wherein in formula (3):
R is C₄-C₁₈ alkyl or benzyl; 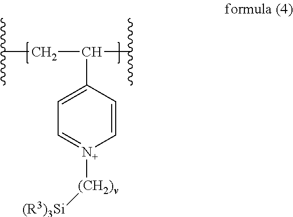
wherein in formula (4):
each R³ is independently optionally substituted alkoxy; and
v is an integer from 3 to 10.

11. The polymer of any one of claims 7-10, wherein in formula (4), v is 3 and each R³ is methoxy.

12. The polymer of any one of claims 7-11, wherein the moieties of formula (Ia), moieties of formula (3), and moieties of formula (4) are present in a molar ratio of x:y:z, wherein 0.1≤x≤0.2:0.6≤y≤0.8:0.1≤z≤0.2, and x+y+z=1;
wherein the moieties of formula (Iaa), moieties of formula (3), and moieties of formula (4) are present in a molar ratio of x:y:z, wherein 0.1≤x≤0.2:0.6≤y≤0.8:0.1≤z≤0.2, and x+y+z=1;
wherein the moieties of formula (VIIa), moieties of formula (3), and moieties of formula (4) are present in a molar ratio of x:y:z, wherein 0.1≤x≤0.2:0.6≤y≤0.8:0.1≤z≤0.2, and x+y+z=1; and/or
wherein the moieties of formula (VIIb), moieties of formula (3), and moieties of formula (4) are present in a molar ratio of x:y:z, wherein 0.1≤x≤0.2:0.6≤y≤0.8:0.1≤z≤0.2, and x+y+z=1.

13. The polymer of claim 1, wherein the polymer comprises at least one moiety of formula (Iaa), at least one moiety of formula (5), and at least one moiety of formula (6): 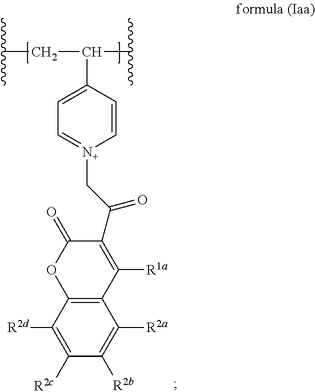
wherein in formula (Iaa):
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; 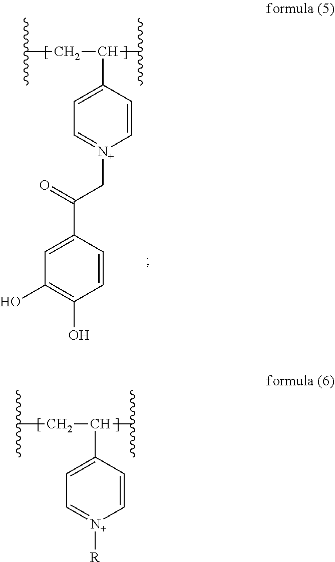
wherein in formula (6):
R is C₄-C₁₈ alkyl or benzyl.

14. The polymer of claim 1 or 13, wherein the polymer comprises at least one moiety of formula (Ia), at least one moiety of formula (5), and at least one moiety of formula (6): 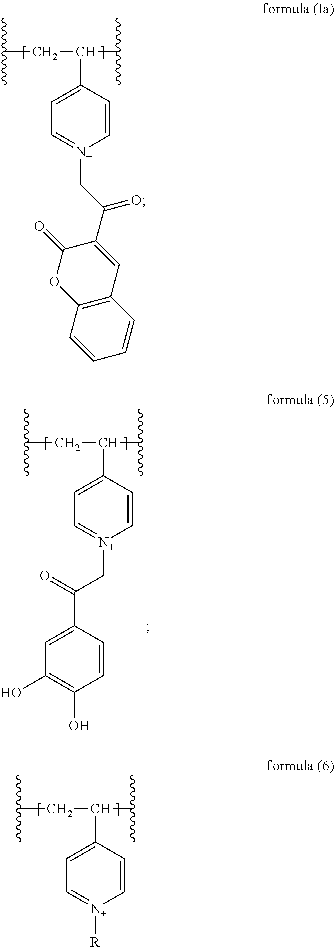
wherein in formula (6):
R is C₄-C₁₈ alkyl or benzyl.

15. The polymer of claim 1, wherein the polymer comprises at least one moiety of formula (VIIa), at least one moiety of formula (5), and at least one moiety of formula (6): 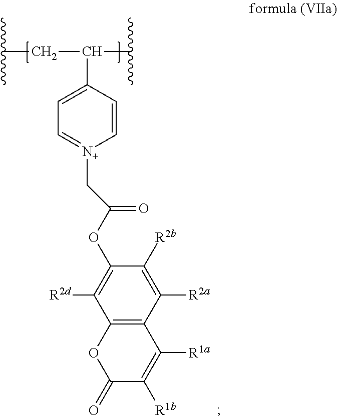
wherein in formula (VIIa):
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; 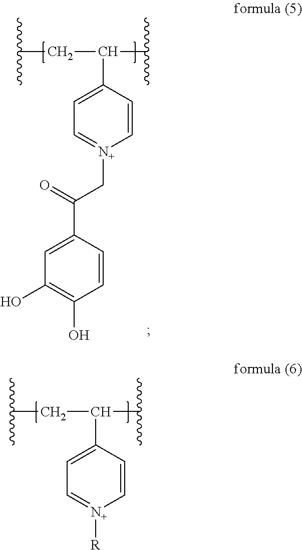
wherein in formula (6):
R is C₄-C₁₈ alkyl or benzyl.

16. The polymer of claim 1 or 15, wherein the polymer comprises at least one moiety of formula (VIIb), at least one moiety of formula (5), and at least one moiety of formula (6): 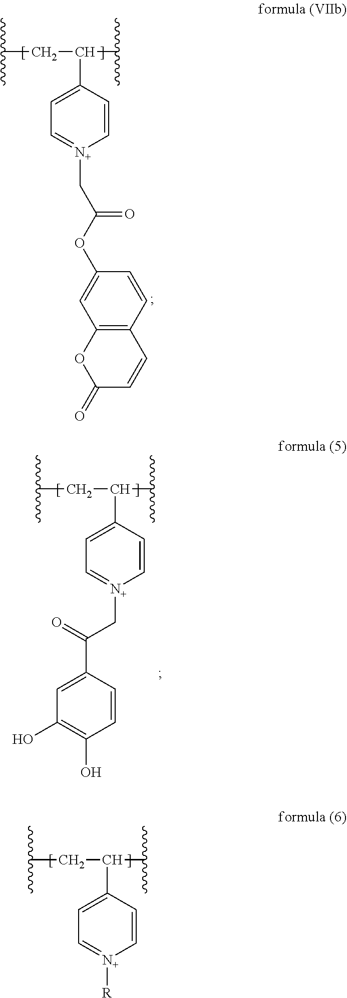
wherein in formula (6):
R is C₄-C₁₈ alkyl or benzyl.

17. The polymer of any one of claims 13-16, wherein the moieties of formula (Iaa), moieties of formula (5), and moieties of formula (6) are present in a molar ratio of x:y:z, wherein 0.1≤x≤0.2:0.1≤y≤0.2:0.6≤z≤0.8, and x+y+z=1;
the moieties of formula (Ia), moieties of formula (5), and moieties of formula (6) are present in a molar ratio of x:y:z, wherein 0.1≤x≤0.2:0.1≤y≤0.2:0.6≤z≤0.8, and x+y+z=1;
the moieties of formula (VIIa), moieties of formula (5), and moieties of formula (6) are present in a molar ratio of x:y:z, wherein 0.1≤x≤0.2:0.1≤y≤0.2:0.6≤z≤0.8, and x+y+z=1;
and/or the moieties of formula (VIIb), moieties of formula (5), and moieties of formula (6) are present in a molar ratio of x:y:z, wherein 0.1≤x≤0.2:0.1≤y≤0.2:0.6≤z≤0.8, and x+y+z=1.18.
The polymer of claim 1, wherein the polymer comprises at least one moiety of formula (Ibb) at least one moiety of formula (7), and at least one moiety of formula (8): 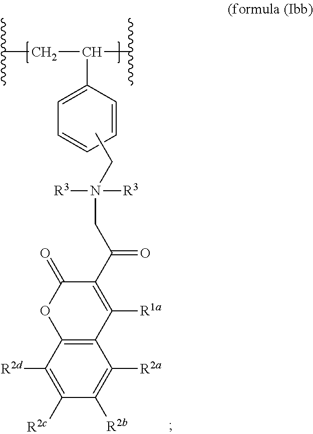
wherein in formula (Ibb):
R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkyl; 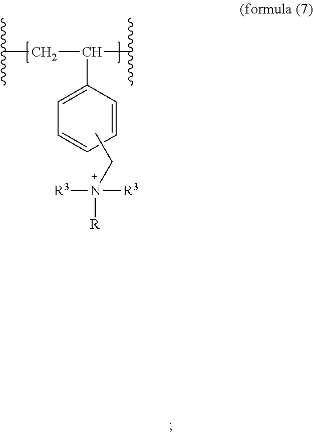
wherein in formula (7):
each R³ is independently optionally substituted alkyl; and
R is C₄-C₁₈ alkyl or benzyl; 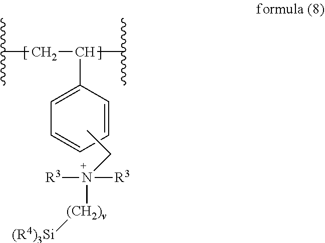
wherein in formula (8):
each R³ is independently optionally substituted alkyl;
each R⁴ is independently optionally substituted alkoxy; and
v is an integer from 3 to 10.

19. The polymer of claim 1 or 18, wherein the polymer comprises at least one moiety of formula (Ib) at least one moiety of formula (7), and at least one moiety of formula (8): 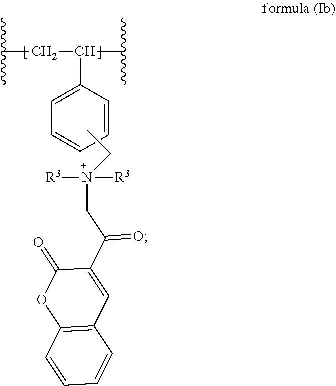
wherein in formula (Ib):
each R³ is independently optionally substituted alkyl; 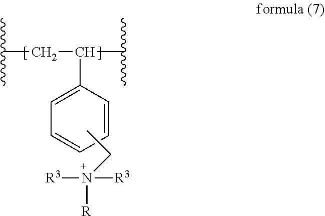
wherein in formula (7):
each R³ is independently optionally substituted alkyl; and
R is C₄-C₁₈ alkyl or benzyl; 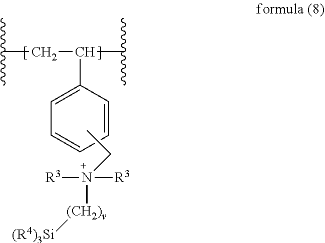
wherein in formula (8):
each R³ is independently optionally substituted alkyl;
each R⁴ is independently optionally substituted alkoxy; and
v is an integer from 3 to 10.

20. The polymer of claim 1, wherein the polymer comprises at least one moiety of formula (IIaa), at least one moiety of formula (7), and at least one moiety of formula (8): 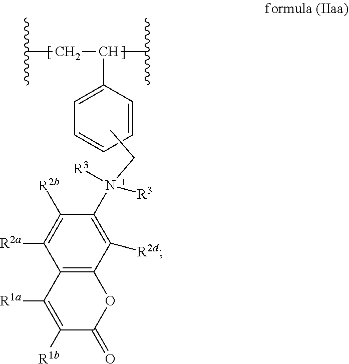
wherein in formula (IIaa):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkyl; 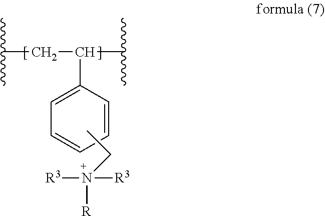
wherein in formula (7):
each R³ is independently optionally substituted alkyl; and
R is C₄-C₁₈ alkyl or benzyl; 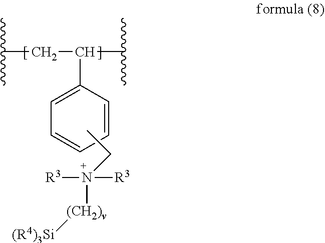
wherein in formula (8):
each R³ is independently optionally substituted alkyl;
each R⁴ is independently optionally substituted alkoxy; and
v is an integer from 3 to 10.

21. The polymer of claim 1 or 20, wherein the polymer comprises at least one moiety of formula (IIa), at least one moiety of formula (7), and at least one moiety of formula (8): 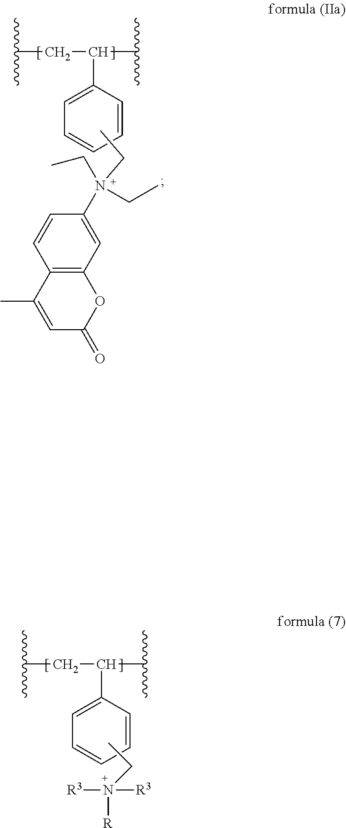
wherein in formula (7):
each R³ is independently optionally substituted alkyl; and
R is C₄-C₁₈ alkyl or benzyl; 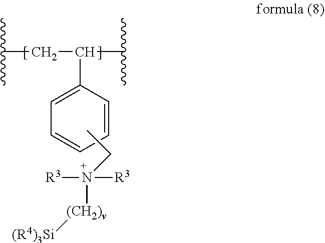
wherein in formula (8):
each R³ is independently optionally substituted alkyl;
each R⁴ is independently optionally substituted alkoxy; and
v is an integer from 3 to 10.

22. The polymer of claim 19, wherein in formula (Ib), each R³ is ethyl.

23. The polymer of any one of claims 18-23, wherein in formula (7), each R³ is methyl.

24. The polymer of any one of claims 18-24, wherein in formula (8), each R³ is methyl, v is 3, and each R⁴ is methoxy.

25. The polymer of any one of claims 12-15, wherein the moieties of formula (Ibb), moieties of formula (7), and moieties of formula (8) are present in a molar ratio of x:y:z, wherein 0.1≤x≤0.2:0.6≤y≤0.8:0.1≤z≤0.2, and x+y+z=1;
the moieties of formula (Ibb), moieties of formula (7), and moieties of formula (8) are present in a molar ratio of x:y:z, wherein 0.1≤x≤0.2:0.6≤y≤0.8:0.1≤z≤0.2, and x+y+z=1;
the moieties of formula (IIaa), moieties of formula (7), and moieties of formula (8) are present in a molar ratio of x:y:z, wherein 0.1≤x≤0.2:0.6≤y≤0.8:0.1≤z≤0.2, and x+y+z=1; and/or
the moieties of formula (IIa), moieties of formula (7), and moieties of formula (8) are present in a molar ratio of x:y:z, wherein 0.1≤x≤0.2:0.6≤y≤0.8:0.1≤z≤0.2, and x+y+z=1.

26. The polymer of claim 1, wherein the polymer comprises at least one moiety of formula (IIaa), at least one moiety of formula (7), and at least one moiety of formula (13): 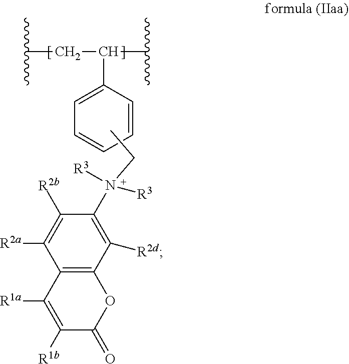
wherein in formula (IIaa):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkyl; 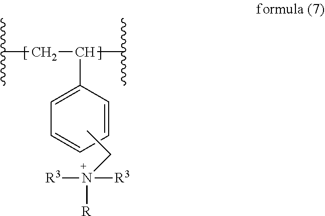
wherein in formula (7):
each R³ is independently optionally substituted alkyl; and
R is C₄-C₁₈ alkyl or benzyl; 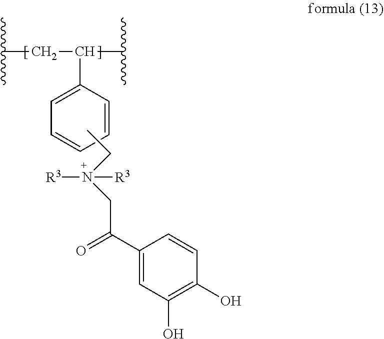
wherein in formula (13):
each R³ is independently optionally substituted alkyl.

27. The polymer of claim 1 or 26, wherein the polymer comprises at least one moiety of formula (IIa), at least one moiety of formula (7), and at least one moiety of formula (13): 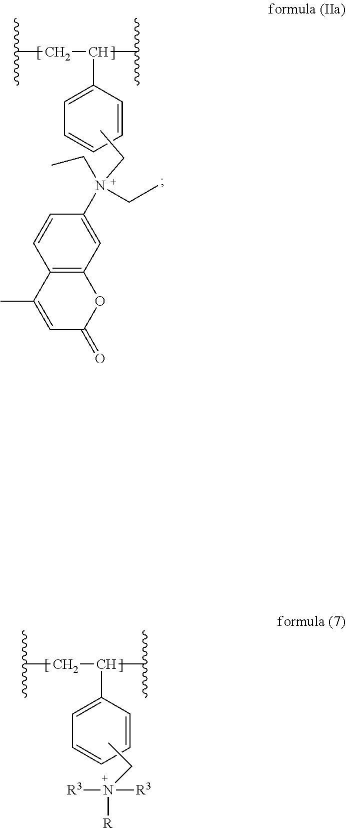
wherein in formula (7):
each R³ is independently optionally substituted alkyl; and
R is C₄-C₁₈ alkyl or benzyl; 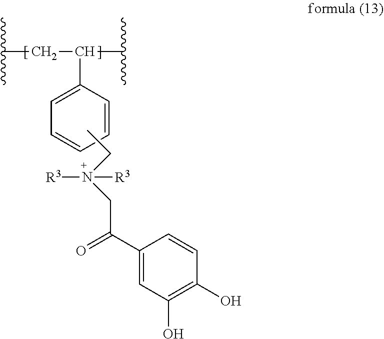
wherein in formula (13):
each R³ is independently optionally substituted alkyl.

27. The polymer of any one of claims 25 and 26, wherein the moieties of formula (IIaa), moieties of formula (7), and moieties of formula (13) are present in a molar ratio of x:y:z, wherein 0.1≤x≤0.2:0.6≤y≤0.8:0.1≤z≤0.2, and x+y+z=1; and/or
the moieties of formula (IIa), moieties of formula (7), and moieties of formula (13) are present in a molar ratio of x:y:z, wherein 0.1≤x≤0.2:0.6≤y≤0.8:0.1≤z≤0.2, and x+y+z=1.

28. The polymer of any one of claims 1-3, wherein the polymer comprises a polyethylenimine (PEI) polymer comprising at least one moiety of formula (IXa), formula (IXb), or formula (IXc): 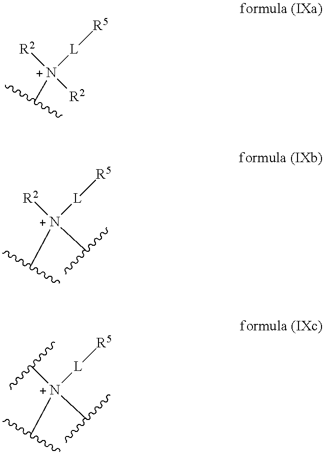
wherein in formula (IXa), formula (IXb), and formula (IXc):
L is a single bond or a linking group;
R⁵ is a detectable moiety; and
each R² is independently optionally substituted alkyl.

29. The polymer of claim 28, wherein L is selected from

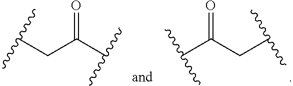 [SMILES: *CC(*)=O ; *CC(*)=O]

30. The polymer of claim 28, wherein the PEI polymer comprises at least one moiety of formula (IXa1), formula (IXb1), or formula (IXc1): 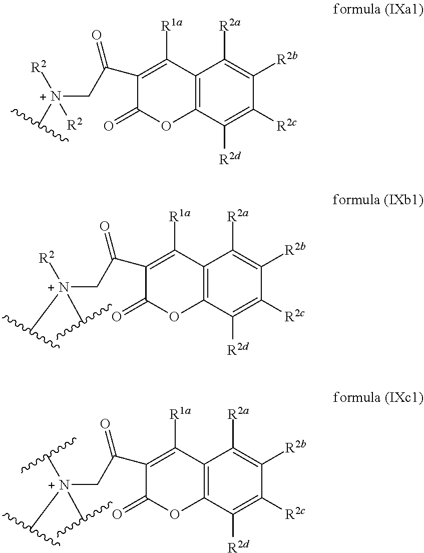
wherein in formula (IXa1), formula (IXb1), and formula (IXc1):
each R² is independently optionally substituted alkyl;
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl, optionally wherein each R^{2a}, R^{2b}, R^{2c}, and R^{2d} is hydrogen.

31. The polymer of claim 28, wherein the PEI polymer comprises at least one moiety of formula (IXa2), formula (IXb2), or formula (IXc2): 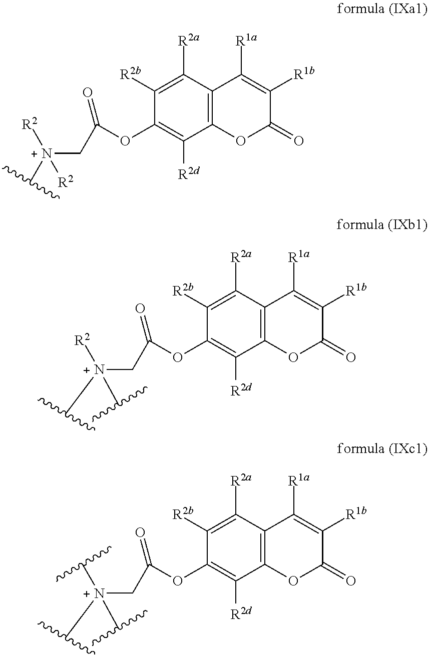
wherein in formula (IXa2), formula (IXb2), and formula (IXc2):
each R² is independently optionally substituted alkyl;
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl, optionally wherein each R^{1a}, R^{1b}, R^{2a}, R^{2b}, and R^{2d} is hydrogen.

32. The polymer of any one of claims 28-31, wherein the PEI polymer is fully quaternized, optionally fully methylated.

33. The polymer of any one of claims 28-32, wherein the PEI polymer further comprises one or more optionally substituted C₄-C₂₂ alkyl groups, optionally C₆ alkyl groups or C₁₀ alkyl group, and one or more 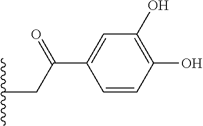 [SMILES: *CC(=O)c1ccc(O)c(O)c1]
groups.

34. The polymer of claim 33, wherein the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number of 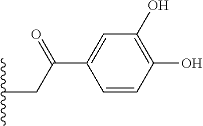 [SMILES: *CC(=O)c1ccc(O)c(O)c1]
groups is x:y:z, wherein about 0.05≤x≤0.4:0.2≤y≤0.9:0.05≤z≤0.4, optionally 0.1:0.8:0.1.

35. The polymer of any one of claims 28-32, wherein the PEI polymer further comprises one or more optionally substituted C₄-C₂₂ alkyl groups, optionally C₆ alkyl groups or C₁₀ alkyl group, and one or more moieties of formula (9): 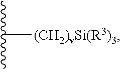
wherein each R³ is independently optionally substituted alkoxy; and v is an integer from 3 to 10, optionally

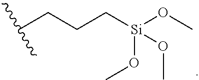 [SMILES: *CCC[Si](OC)(OC)OC]

36. The polymer of claim 35, wherein the number of moieties of formula (IXa), (IXa1) and/or (IXa2) and/or one or more moieties of formula (IXb), (IXb1) and/or (IXb2), and/or one or more moieties of formula (IXc), (IXc1) and/or (IXc2) to the number of optionally substituted C₄-C₂₂ alkyl groups to the number one or more moieties of formula (9), optionally 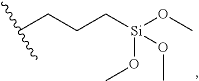 [SMILES: *CCC[Si](OC)(OC)OC]
is x:y:z, wherein about 0.05≤x≤0.4:0.2≤y≤0.9:0.05≤z≤0.4, optionally 0.1:0.8:0.1.

37. The polymer of any one of claims 28-31, wherein the PEI polymer a PEI polymer comprises at least one moiety of formula (IXe), or substructures thereof: 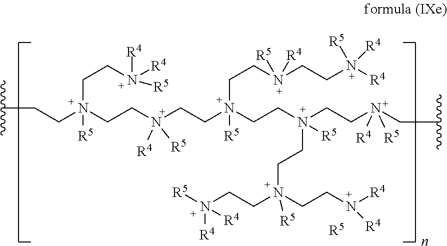
wherein in formula (IXe):
each R⁴ is independently optionally substituted alkyl; and
each R⁵ is independently optionally substituted alkyl, a moiety of formula (I): 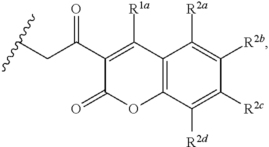
R^{2d} or a catechol moiety of formula (11): 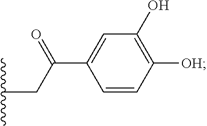 [SMILES: *CC(=O)c1ccc(O)c(O)c1]
R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl;
with the proviso that at least one R⁵ is a moiety of formula (I):

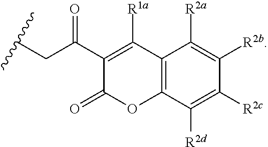

38. The polymer of any one of claims 28-31, wherein the PEI polymer a PEI polymer comprises at least one moiety of formula (IXf), or substructures thereof: 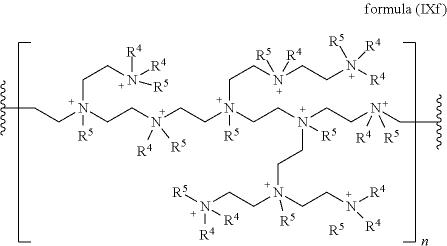
wherein in formula (IXf):
each R⁴ is independently optionally substituted alkyl; and
each R⁵ is independently optionally substituted alkyl, a moiety of formula (VII): 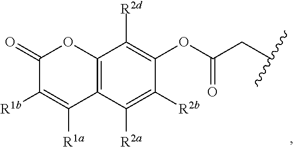
or a catechol moiety of 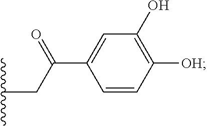 [SMILES: *CC(=O)c1ccc(O)c(O)c1]
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl;
with the proviso that at least one R⁵ is a moiety of formula (VIII):

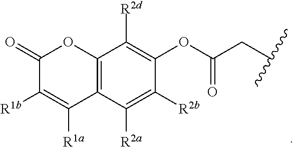

39. The polymer of any one of claims 28-31, wherein the PEI polymer a PEI polymer comprises at least one moiety of formula (IXg), or substructures thereof: 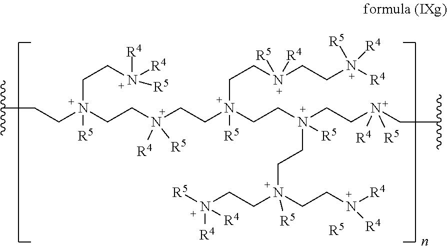
wherein in formula (IXg):
each R⁴ is independently optionally substituted alkyl; and
each R⁵ is independently optionally substituted alkyl, a moiety of formula (I): 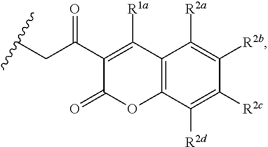
or a silane moiety of formula (9): 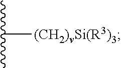
R^{1a} is hydrogen or alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R³ is independently optionally substituted alkoxy; and
v is an integer from 3 to 10;
with the proviso that at least one R⁵ is a moiety of formula (I):

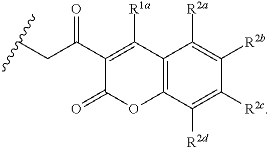

40. The polymer of any one of claims 28-31, wherein the PEI polymer a PEI polymer comprises at least one moiety of formula (IXh), or substructures thereof: 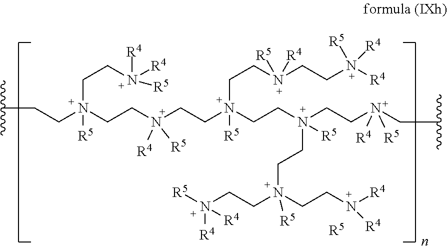
wherein in formula (IXh):
each R⁴ is independently optionally substituted alkyl; and
each R⁵ is independently optionally substituted alkyl, a moiety of formula (VII): 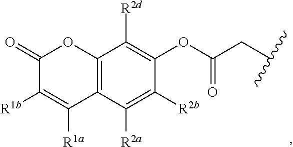
or a silane moiety of formula (9): 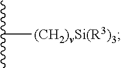
each R^{1a} and R^{1b} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R³ is independently optionally substituted alkoxy; and
v is an integer from 3 to 10;
with the proviso that at least one R⁵ is a moiety of formula (VIII):

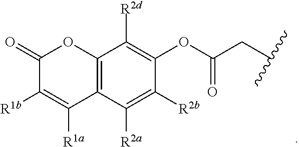

41. The polymer of any one of claims 37-40, wherein in formula (IXg), the molar ratio of the number of R⁵ moieties of formula (I) to the number of R⁵ optionally substituted alkyl groups to the number of R⁵ catechol moieties of 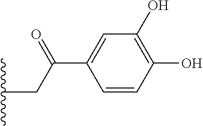 [SMILES: *CC(=O)c1ccc(O)c(O)c1]
is about 0.1:0.8:0.1;
wherein in formula (IXf), the molar ratio of the number of R⁵ moieties of formula (VII) to the number of R⁵ optionally substituted alkyl groups to the number of R⁵ catechol moieties of 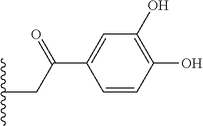 [SMILES: *CC(=O)c1ccc(O)c(O)c1]
is about 0.1:0.8:0.1;
wherein in formula (IXg), the molar ratio of the number of R⁵ moieties of formula (I) to the number of R⁵ optionally substituted alkyl groups to the number of R⁵ groups of formula (9) is about 0.1:0.8:0.1; and/or
wherein in formula (IXh), the molar ratio of the number of R⁵ moieties of formula (VII) to the number of R⁵ optionally substituted alkyl groups to the number of R⁵ groups of formula (9) is about 0.1:0.8:0.1.

42. A compound comprising one or more detectable moieties.

43. The compound of claim 42, wherein the detectable moiety is one or more moieties selected from a fluorescent moiety, a phosphorescent moiety, and a luminescent moiety.

44. The compound of claim 42, wherein the detectable moiety is a fluorescent moiety selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety.

45. The compound of any of claims 42-44, wherein the detectable moiety is a moiety of formula (I) or formula (II): 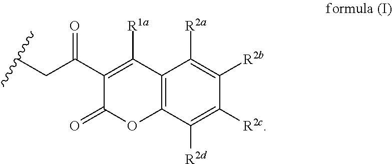
wherein in formula (I):
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; 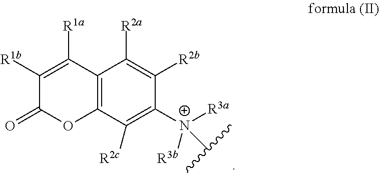
wherein in formula (II):
each R^{1a} and R^{1a} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

46. The compound of claim 45, wherein the detectable moiety is selected from formula (10) and formula (20): 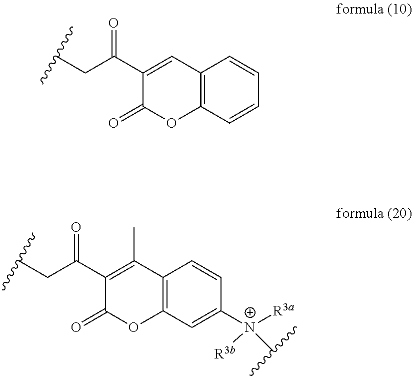
wherein in formula (20) R^{3a} and R^{3b} are each independently optionally substituted alkyl.

47. The compound of claim 45 or 46, wherein R^{3a} and R^{3b} are each ethyl.

48. The compound of claim 45, wherein the compound of formula (I) is a compound of formula (Ic): 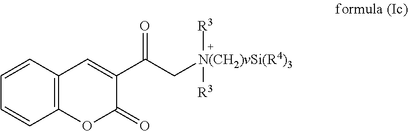
wherein in formula (XVII):
each R³ is independently optionally substituted alkyl;
each R⁴ is independently optionally substituted alkoxy; and
v is an integer from 3 to 10.

49. The compound of claim 48, wherein each R³ is methyl, v is 3, and each R⁴ is methoxy.

50. The compound of claim 45, wherein the compound of formula (II) is a compound of formula (IIa): 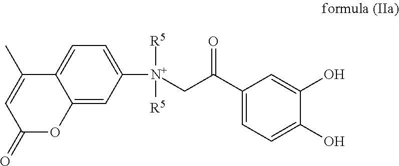
wherein in formula (IIa):
each R⁵ is independently optionally substituted alkyl.

51. The compound of claim 50, wherein each R⁵ is ethyl.

52. The compound of any of claims 42-44, wherein the compound is a compound having formula (III): 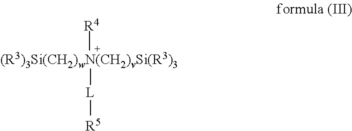
wherein in formula (III):
L is a single bond or a linking group;
each R³ is independently optionally substituted alkoxy;
R⁴ is independently optionally substituted alkyl;
R⁵ is a detectable moiety;
v is an integer from 3 to 10; and
w is an integer from 3 to 10.

53. The compound of claim 52, wherein the compound of formula (III) is a compound having formula (IIIa) or formula (IIIb): 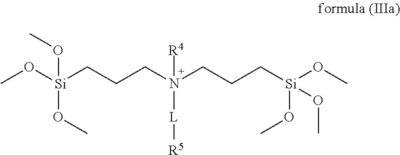
wherein in formula (IIIa):
L is a single bond or a linking group;
R⁴ is optionally substituted alkyl; and
R⁵ is a detectable moiety; 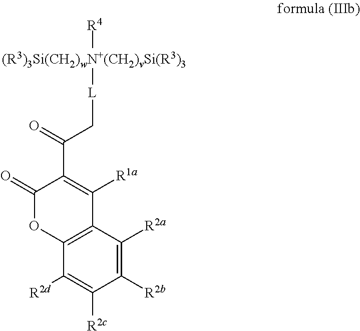
wherein in formula (IIIb):
L is a single bond or a linking group;
R^{1a} is independently optionally selected from hydrogen and alkyl;
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R³ is independently optionally substituted alkoxy; and
R⁴ is optionally substituted alkyl.

54. The compound of claim 52 or 53, wherein the compound has the formula (300) or formula (301):

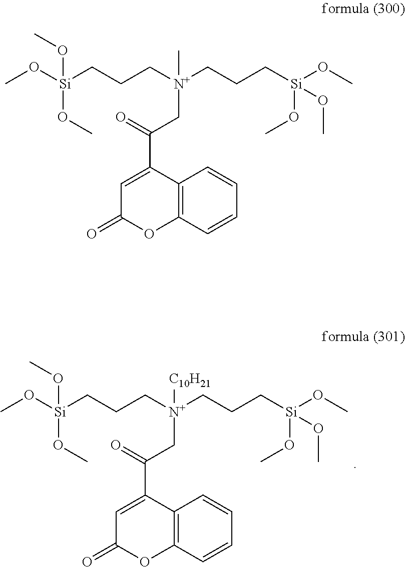

55. The compound of any of claims 42-44, wherein the compound is a compound of formula (IV): 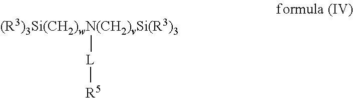
wherein in formula (IV):
L is a single bond or a linking group;
each R³ is independently optionally substituted alkoxy;
R⁵ is a detectable moiety;
v is an integer from 3 to 10; and
w is an integer from 3 to 10.

56. The compound of claim 55, wherein the compound of formula (IV) is a compound of formula (IVa) or formula (IVb): 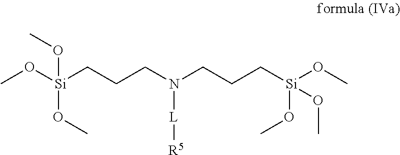
wherein in formula (IVa):
L is a single bond or a linking group; and
R⁵ is a detectable moiety; 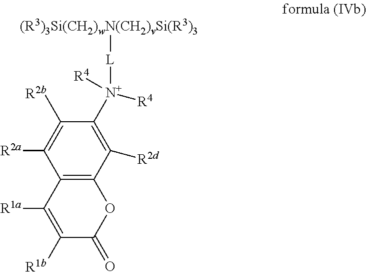
wherein in formula (IVb):
L is a single bond or a linking group;
each R³ is independently optionally substituted alkoxy;
each R⁴ is independently optionally substituted alkyl;
v is an integer from 3 to 10; and
w is an integer from 3 to 10.

57. The compound of claim 55 or 56, wherein the compound has formula (400):

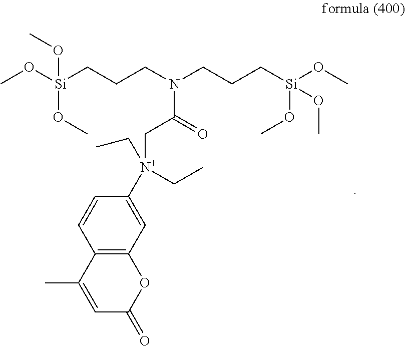

58. The compound of any of claims 42-44, wherein the compound is a compound having formula (VI): 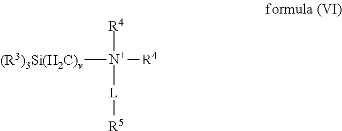
wherein in formula (VI):
L is a single bond or a linking group;
each R³ is independently optionally substituted alkoxy;
each R⁴ is independently optionally substituted alkyl;
R⁵ is a detectable moiety; and
v is an integer from 3 to 10.

59. The compound of claim 58, wherein the compound of formula (VI) is a compound having formula (VIa) or formula (VIb): 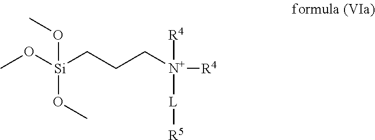
wherein in formula (VIa):
L is a single bond or a linker;
each R⁴ is independently optionally substituted alkyl; and
R⁵ is a detectable moiety; 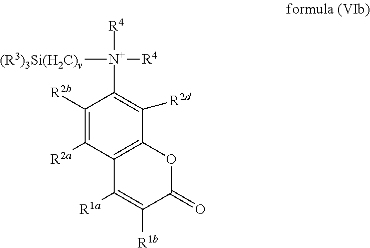
wherein in formula (VIb):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl; and
each R³ is independently optionally substituted alkoxy;
each R⁴ is independently optionally substituted alkyl; and
v is an integer from 3 to 10.

60. The compound of claim 58 or 59, wherein the compound has the formula (600) or formula (601):

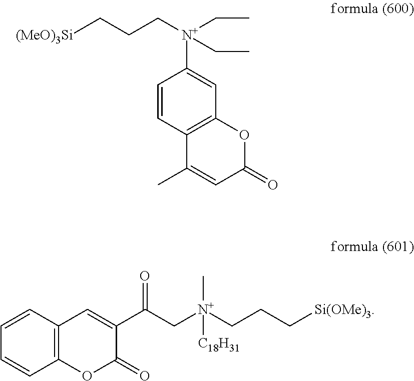

61. The compound of any of claims 42-44, wherein the compound is a compound having formula (VIII): 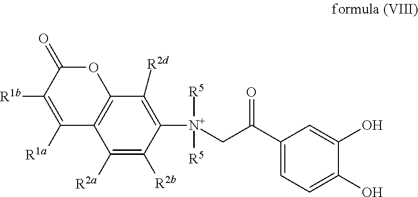
wherein in formula (VIII):
each R^{1a} and R^{1b} is independently optionally selected from hydrogen and alkyl;
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl;
each R⁵ is independently optionally substituted alkyl.

62. The compound of claim 61, wherein the compound of formula (VIII) is a compound having formula (VIIIa): 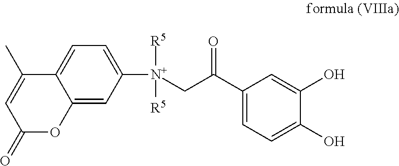
wherein in formula (VIIIa):
each R⁵ is independently optionally substituted alkyl.

63. The compound of claim 61 or 62, wherein the compound has formula (800):

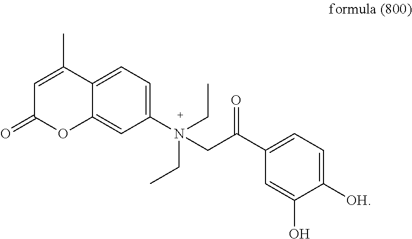

64. A method of preparing a polymer comprising a detectable moiety, the method comprising treating a precursor polymer with a reactive detectable compound.

65. The method of claim 64, wherein the detectable compound is covalently attached to the precursor polymer after treatment.

66. The method of claim 64 or 65, wherein the detectable moiety comprises one or more moieties selected from a fluorescent moiety, a phosphorescent moiety, and a luminescent moiety.

67. The method of claim 66, wherein the fluorescent moiety is selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety.

68. The method of claim 66, wherein the reactive detectable compound is a compound of formula (100) or formula (200): 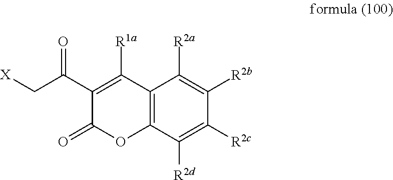
wherein in formula (100):
X is halogen;
R^{1a} is hydrogen or alkyl; and
each R^{2a}, R^{2b}, R^{2c}, and R^{2d} is independently optionally selected from hydrogen, halogen, —OH, optionally substituted alkyl, optionally substituted amino, optionally substituted alkoxy, optionally substituted aryl, and optionally substituted heteroaryl; 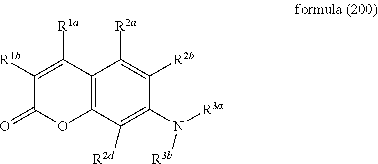
wherein in formula (200):
each R^{1a} and R^{1a} is independently optionally selected from hydrogen and alkyl; and
each R^{2a}, R^{2b}, and R^{2d} is independently optionally selected from hydrogen, halogen, —OH, optionally substituted alkyl, optionally substituted amino, optionally substituted alkoxy, optionally substituted aryl, and optionally substituted heteroaryl.

69. A method of preparing a detectable coating, the method comprising depositing a biocidal polymer solution comprising one or more polymers of any one of claims 1-41 onto a surface.

70. The method of claim 69, wherein the method comprises waiting a suitable period of time for the biocidal polymer solution to dry.

71. A method of preparing a detectable coating, the method comprising depositing a biocidal polymer solution onto a surface, wherein the biocidal polymer solution comprises one or more polymers and one or more compounds of claims 42-63.

72. The method of claim 71, wherein the method comprises waiting a period of time for the biocidal polymer solution to dry.

73. The method of claim 72, wherein the compound is cross-linked to the polymer after drying.

74. A detectable coating comprising one or more polymers of any one of claims 1-41.

75. A detectable coating comprising one or more polymers and one or more compounds of any one of claims 42-63, optionally wherein the one or more polymers and the one or more compounds are present in a ratio of about 1:1 w/w.

76. The detectable coating of claim 71 or 75, wherein the polymer is selected from PVP-silane-coalkyl-PVP and PVP-catechol-coalkyl-PVP.

77. The detectable coating of any one of claims 74-76, wherein the detectable coating emits light when exposed to light having a wavelength ranging from about 10 nm to about 400 nm.
